SEQUENCE LISTING (74 sequences)

SEQ ID NO: 1 - DNA, 357 bp, synthetic construct
  misc_feature 1..357: note = source = /note="Description of Artificial Sequence: Synthetic polynucleotide"
  source 1..357: mol_type = other DNA
caggtgcagctgcagcagtggggtgccggtctgctgaagccttctgaaactctgtctctgacttgtgccgtctatggtggatcattcagcggctactattggtcctggatcaggcagccccctggcaagggcctggagtggatcggcgagatcaaccaccggggctctaccaactacaatccctctctgaagagcagggtgaccatctccgtggacacatctaagaatcagttcagcctgaagctgagctccgtgaccgccgctgatacagccgtgtactattgcacaaggggggaggaatgggagtcactgttctttgattactgggggcaggggacactggtcacagtctcgagt

SEQ ID NO: 2 - DNA, 321 bp, synthetic construct
  misc_feature 1..321: note = source = /note="Description of Artificial Sequence: Synthetic polynucleotide"
  source 1..321: mol_type = other DNA
gaaatcgtcctgacccagtcccccgccaccctgagcctgagccccggagaaagagccaccctgtcctgccgagcaagccagtccatcagctcctatctcgcctggtatcagcagaaaccaggccaggctccccggctgctgatctacggcgcctccaacagagctacaggaatcccagcccgcttcagcggctccggctctggcacagactttaccctgacaatctctagcctggagcctgaggatttcgccgtgtactattgccagcagagatctaattggccactgacattcggcggcggcacacgggtggagatcaag

SEQ ID NO: 3 - protein, 119 aa, synthetic construct
  REGION 1..119: note = source = /note="Description of Artificial Sequence: Synthetic polypeptide"
QVQLQQWGAGLLKPSETLSLTCAVYGGSFSGYYWSWIRQPPGKGLEWIGEINHRGSTNYNPSLKSRVTISVDTSKNQFSLKLSSVTAADTAVYYCTRGEEWESLFFDYWGQGTLVTVSS

SEQ ID NO: 4 - protein, 107 aa, synthetic construct
  REGION 1..107: note = source = /note="Description of Artificial Sequence: Synthetic polypeptide"
EIVLTQSPATLSLSPGERATLSCRASQSISSYLAWYQQKPGQAPRLLIYGASNRATGIPARFSGSGSGTDFTLTISSLEPEDFAVYYCQQRSNWPLTFGGGTRVEIK

SEQ ID NO: 5 - DNA, 366 bp, synthetic construct
  misc_feature 1..366: note = source = /note="Description of Artificial Sequence: Synthetic polynucleotide"
  source 1..366: mol_type = other DNA
caggttcagctgcagcagtggggcgccggcctgctgagaccaagcgagaccctgtccctgacatgcgccgtgtatggcgagagcttctccggctattactggaactggatccggcagcctcccggcaagggcctggagtggatcggcgagatcaatcactccggctccaccaattacaacccatccctgaagtctcgggtgacaatcagcgtggatacaagcaagacccagttcagcctgaagctgagctccgtgacagctgccgataccgccgtgtattactgcgccagaggctgggacctgctggattggaatgactactggaatgagtactggggccaggggaccctggtgaccgtctcgagt

SEQ ID NO: 6 - DNA, 321 bp, synthetic construct
  misc_feature 1..321: note = source = /note="Description of Artificial Sequence: Synthetic polynucleotide"
  source 1..321: mol_type = other DNA
gagatcgtgctgacccagtcccctgccaccctgtctctgtcccctggcgagcgggccaccctgtcctgtagagcttctcagtccgtgtcttcctacctggcttggtaccagcagaagccaggacaggccccaagactgctgatctatgacgcttccaatcgggctaccggcatcccagctcgctttagcggctccggctccggcaccgacttcaccctgacaatcagctccctggagccagaggattttgccgtgtattactgtcagcagaggtccaattggccactgacatttggcggcggcacaaaggttgagatcaag

SEQ ID NO: 7 - protein, 122 aa, synthetic construct
  REGION 1..122: note = source = /note="Description of Artificial Sequence: Synthetic polypeptide"
QVQLQQWGAGLLRPSETLSLTCAVYGESFSGYYWNWIRQPPGKGLEWIGEINHSGSTNYNPSLKSRVTISVDTSKTQFSLKLSSVTAADTAVYYCARGWDLLDWNDYWNEYWGQGTLVTVSS

SEQ ID NO: 8 - protein, 107 aa, synthetic construct
  REGION 1..107: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 9 - DNA, 357 bp, synthetic construct
  misc_feature 1..357: note = source = /note="Description of Artificial Sequence: Synthetic polynucleotide"
  source 1..357: mol_type = other DNA
caggttcagctgcagcagtggggcgctggcctgctgaagccctctgagaccctgtctctgacctgtgccgtgtatggcggcagcttctccggctattactggaactggatccgccagccccccggcaagggcctggagtggatcggcgagatcaaccactccggctctaccaactacaatccttctctgaagtccagggtgacaatcagcgtggacaccagcaagaaccagtttagcctgaagctgtccagcgtgacagctgccgatacagccgtgtattactgcgccagaggcgaggattggggcgagagcttctttgattactggggccaggggaccctggtgaccgtctcgagt

SEQ ID NO: 10 - DNA, 321 bp, synthetic construct
  misc_feature 1..321: note = source = /note="Description of Artificial Sequence: Synthetic polynucleotide"
  source 1..321: mol_type = other DNA
gagatcgtgctgacccagtcccctgccaccctgtctctgtcccctggcgagcgggccaccctgagctgtcgggcctcccagtccgtgagctcctacctggcttggtatcagcagaagccaggacaggccccaagactgctgatctacgccgcttccaatcgggccaccggcatccccgccagattttccggctctggctccggcaccgatttcaccctgacaatcagctccctggagccagaggacttcgccgtgtactactgccagcagaggtctaattggccactgacatttggcggcggcaccaaggttgagatcaag

SEQ ID NO: 11 - protein, 119 aa, synthetic construct
  REGION 1..119: note = source = /note="Description of Artificial Sequence: Synthetic polypeptide"
QVQLQQWGAGLLKPSETLSLTCAVYGGSFSGYYWNWIRQPPGKGLEWIGEINHSGSTNYNPSLKSRVTISVDTSKNQFSLKLSSVTAADTAVYYCARGEDWGESFFDYWGQGTLVTVSS

SEQ ID NO: 12 - protein, 107 aa, synthetic construct
  REGION 1..107: note = source = /note="Description of Artificial Sequence: Synthetic polypeptide"
EIVLTQSPATLSLSPGERATLSCRASQSVSSYLAWYQQKPGQAPRLLIYAASNRATGIPARFSGSGSGTDFTLTISSLEPEDFAVYYCQQRSNWPLTFGGGTKVEIK

SEQ ID NO: 13 - DNA, 357 bp, synthetic construct
  misc_feature 1..357: note = source = /note="Description of Artificial Sequence: Synthetic polynucleotide"
  source 1..357: mol_type = other DNA
caggttcagctggtgcagtccggcgctgaggtgaagaagcctggagcttctgtgaaggtttcctgtaaggtttccggctatagcctgaccgagatctctatgcactgggtacggcaagcccccggcaagggcctggagtggatgggcggctttgacccagaggatggcgagaccatctacgctcagaggtttcaggggcgcgtgatcatgaccgaggataccagcaccgataccgcctacatggagctgtccagcctgagatccgaggataccgccgtgtattactgcgctactggtggctggggccccaattggttcgatccttggggccaggggaccctggtgaccgtctcgagt

SEQ ID NO: 14 - DNA, 321 bp, synthetic construct
  misc_feature 1..321: note = source = /note="Description of Artificial Sequence: Synthetic polynucleotide"
  source 1..321: mol_type = other DNA
gacatccagatgacacagtctccttcctctgtgagcgcctctgtgggcgaccgcgtgaccatcacatgccgcgcttcccaggggatctcttcctggctggcttggtaccagcagaagcccggcaaggcccccaagctgctgatctatgctgccagcagcctgcagtccggcgtgccttccaggtttagcggctccggctccggcaccgactttacactgacaatcagctccctgcagcccgaggattttgccacctattactgtcagcaggcgaattccttcccttttacattcggccctggcaccaaggttgatatcaag

SEQ ID NO: 15 - protein, 119 aa, synthetic construct
  REGION 1..119: note = source = /note="Description of Artificial Sequence: Synthetic polypeptide"
QVQLVQSGAEVKKPGASVKVSCKVSGYSLTEISMHWVRQAPGKGLEWMGGFDPEDGETIYAQRFQGRVIMTEDTSTDTAYMELSSLRSEDTAVYYCATGGWGPNWFDPWGQGTLVTVSS

SEQ ID NO: 16 - protein, 107 aa, synthetic construct
  REGION 1..107: note = source = /note="Description of Artificial Sequence: Synthetic polypeptide"
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIK

SEQ ID NO: 17 - DNA, 363 bp, synthetic construct
  misc_feature 1..363: note = source = /note="Description of Artificial Sequence: Synthetic polynucleotide"
  source 1..363: mol_type = other DNA
caggttcagctgcagcagtccggccccggcctggtgaagccaagccagaccctgtctctgacatgcgccatctccggcgacagcgtgtcctctaactccgccgcttggaattggatccggcagtctccatccagaggcctggagtggctgggcagaacctattaccggtccaagtggtacaacgattatgccgtgtccgtgaagagcagaatcaccatcaaccctgataccagcaagaaccagttcagcctgcagctgaattccgtgaccccagaggatacagccgtgtattactgtgctagggacgatgattggaatgacttcgattactggggccaggggaccctggtgacagtctcgagt

SEQ ID NO: 18 - DNA, 321 bp, synthetic construct
  misc_feature 1..321: note = source = /note="Description of Artificial Sequence: Synthetic polynucleotide"
  source 1..321: mol_type = other DNA
gagatcgtgctgacccagtccccagctacactgtccctgtctcccggcgagcgggccaccctgagctgtagagcttcccagtccgtgtcttcctatctggcttggtatcagcagaagccaggacaggccccaaggctgctgatctacgacgcctccaatagagccaccggcatcccagctagattttctggctccggctccggcaccgatttcacactgaccatctctagcctggagccagaggattttgctgtatattactgccagcagcgcagcaactggcccctgacatttggcggcggcaccaaggttgagatcaag

SEQ ID NO: 19 - protein, 121 aa, synthetic construct
  REGION 1..121: note = source = /note="Description of Artificial Sequence: Synthetic polypeptide"
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDDDWNDFDYWGQGTLVTVSS

SEQ ID NO: 20 - protein, 107 aa, synthetic construct; identical to SEQ ID NO: 8
  REGION 1..107: note = source = /note="Description of Artificial Sequence: Synthetic polypeptide"

SEQ ID NO: 21 - DNA, 372 bp, synthetic construct
  misc_feature 1..372: note = source = /note="Description of Artificial Sequence: Synthetic polynucleotide"
  source 1..372: mol_type = other DNA
cagctgcagctgcaggaaagcggccccggcctggtgaagccctctgagaccctgtccctgacatgcaccgtgagcggcgattccatcagctcttccagctattactggggctggatccggcagccccccggcaagggcctggagtggatcggcagcatcttctactccggcaatacatattataatccttctctgaagagcagggtgacaatcagcgtggatacctccaagaatcagtttagcctgaagctgagctccgtgacagctgccgatacagccgtgtattactgcgctagggaggacgattttctgaccgattattacggcgctttcgacatctggggccaggggacaatggtgacagtctcgagt

SEQ ID NO: 22 - DNA, 318 bp, synthetic construct
  misc_feature 1..318: note = source = /note="Description of Artificial Sequence: Synthetic polynucleotide"
  source 1..318: mol_type = other DNA
gatatccagatgacccagtctccaagcaccctgagcgcctctgtgggcgatcgggtgaccatcacatgtcgggcttctcagtccatcagcagctggctggcttggtatcagcagaagcccggcaaggccccaaagctgctgatctacaaggcctcttccagcgagagcggcgtgccatccaggtttagcggctccggctccggcaccgagtttaccctgaccatctcttccctgcagcccgatgactttgccacctactactgtcagcagtacaattcctatctgacattcggcggcggcaccaaggttgagatcaag

SEQ ID NO: 23 - protein, 124 aa, synthetic construct
  REGION 1..124: note = source = /note="Description of Artificial Sequence: Synthetic polypeptide"
QLQLQESGPGLVKPSETLSLTCTVSGDSISSSSYYWGWIRQPPGKGLEWIGSIFYSGNTYYNPSLKSRVTISVDTSKNQFSLKLSSVTAADTAVYYCAREDDFLTDYYGAFDIWGQGTMVTVSS

SEQ ID NO: 24 - protein, 106 aa, synthetic construct
  REGION 1..106: note = source = /note="Description of Artificial Sequence: Synthetic polypeptide"
DIQMTQSPSTLSASVGDRVTITCRASQSISSWLAWYQQKPGKAPKLLIYKASSSESGVPSRFSGSGSGTEFTLTISSLQPDDFATYYCQQYNSYLTFGGGTKVEIK

SEQ ID NO: 25 - DNA, 375 bp, synthetic construct
  misc_feature 1..375: note = source = /note="Description of Artificial Sequence: Synthetic polynucleotide"
  source 1..375: mol_type = other DNA
gaggtgcagctgctggaatccggaggaggactggtccagccaggtggatccctgcgactgagctgcgccgcttctggcttcgactttagaagctacgcaatgatgtgggtccgccaggcaccaggaaagggactggagtgggtgggagggatcaacggtgaagtcggtggctctaatacatactatgcacctgccgtcaagggaagggctactattagtcgggacaactcaaaaaataccctgtatctgcaaatgaacagtctgagggccgaggataccgccgtgtactattgcgtgaaaggtgctggcgcatgcggcatctgtaatgacgatattgatgcatggggacaggggaccctggtgacagtctcgagt

SEQ ID NO: 26 - DNA, 330 bp, synthetic construct
  misc_feature 1..330: note = source = /note="Description of Artificial Sequence: Synthetic polynucleotide"
  source 1..330: mol_type = other DNA
agttatgagctgactcaggacccagcagtgtcagtcgccctgggccagacagtgagaatcacttgcagtggggctggttcatatgcaggctcctactattacggatggcaccagcagaagcccggacaggcacctgtgacagtcatctacgacaacgataaaaggccaagcaatattcccgaccggttctctgggtccagctctggtaacaccgcctccctgaccattactggggcccaggctgaggacgaagctgattattactgtggctctacaaacgataatgacgatggcggactgtttggctccggaactaaggtcaccgtccta

SEQ ID NO: 27 - protein, 125 aa, synthetic construct
  REGION 1..125: note = source = /note="Description of Artificial Sequence: Synthetic polypeptide"
EVQLLESGGGLVQPGGSLRLSCAASGFDFRSYAMMWVRQAPGKGLEWVGGINGEVGGSNTYYAPAVKGRATISRDNSKNTLYLQMNSLRAEDTAVYYCVKGAGACGICNDDIDAWGQGTLVTVSS

SEQ ID NO: 28 - protein, 110 aa, synthetic construct
  REGION 1..110: note = source = /note="Description of Artificial Sequence: Synthetic polypeptide"
SYELTQDPAVSVALGQTVRITCSGAGSYAGSYYYGWHQQKPGQAPVTVIYDNDKRPSNIPDRFSGSSSGNTASLTITGAQAEDEADYYCGSTNDNDDGGLFGSGTKVTVL

SEQ ID NO: 29 - DNA, 990 bp, synthetic construct
  misc_feature 1..990: note = source = /note="Description of Artificial Sequence: Synthetic polynucleotide"
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 30 - protein, 330 aa, synthetic construct
  REGION 1..330: note = source = /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 318 bp, synthetic construct
  misc_feature 1..318: note = source = /note="Description of Artificial Sequence: Synthetic polynucleotide"
  source 1..318: mol_type = other DNA
ggccagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 32 - protein, 106 aa, synthetic construct
  REGION 1..106: note = source = /note="Description of Artificial Sequence: Synthetic polypeptide"
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 33 - DNA, 321 bp, synthetic construct
  misc_feature 1..321: note = source = /note="Description of Artificial Sequence: Synthetic polynucleotide"
  source 1..321: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 34 - protein, 107 aa, synthetic construct [sequence omitted]

SEQ ID NO: 35 - protein, 8 aa, synthetic construct
  REGION 1..8: note = source = /note="Description of Artificial Sequence: Synthetic peptide"
GGSFSGYY

SEQ ID NO: 36 - protein, 7 aa, synthetic construct
  REGION 1..7: note = source = /note="Description of Artificial Sequence: Synthetic peptide"
INHRGST

SEQ ID NO: 37 - protein, 15 aa, synthetic construct
  REGION 1..15: note = source = /note="Description of Artificial Sequence: Synthetic peptide"
CTRGEEWESLFFDYW

SEQ ID NO: 38 - protein, 6 aa, synthetic construct
  REGION 1..6: note = source = /note="Description of Artificial Sequence: Synthetic peptide"
QSISSY

SEQ ID NO: 39 - skipped

SEQ ID NO: 40 - protein, 11 aa, synthetic construct [sequence omitted]

SEQ ID NO: 41 - protein, 8 aa, synthetic construct
  REGION 1..8: note = source = /note="Description of Artificial Sequence: Synthetic peptide"
GESFSGYY

SEQ ID NO: 42 - protein, 7 aa, synthetic construct
  REGION 1..7: note = source = /note="Description of Artificial Sequence: Synthetic peptide"
INHSGST

SEQ ID NO: 43 - protein, 18 aa, synthetic construct
  REGION 1..18: note = source = /note="Description of Artificial Sequence: Synthetic peptide"
CARGWDLLDWNDYWNEYW

SEQ ID NO: 44 - protein, 6 aa, synthetic construct
  REGION 1..6: note = source = /note="Description of Artificial Sequence: Synthetic peptide"
QSVSSY

SEQ ID NO: 45 - skipped

SEQ ID NO: 46 - protein, 15 aa, synthetic construct
  REGION 1..15: note = source = /note="Description of Artificial Sequence: Synthetic peptide"
CARGEDWGESFFDYW

SEQ ID NO: 47 - skipped

SEQ ID NO: 48 - protein, 8 aa, synthetic construct
  REGION 1..8: note = source = /note="Description of Artificial Sequence: Synthetic peptide"
GYSLTEIS

SEQ ID NO: 49 - protein, 8 aa, synthetic construct
  REGION 1..8: note = source = /note="Description of Artificial Sequence: Synthetic peptide"
FDPEDGET

SEQ ID NO: 50 - protein, 14 aa, synthetic construct
  REGION 1..14: note = source = /note="Description of Artificial Sequence: Synthetic peptide"
CATGGWGPNWFDPW

SEQ ID NO: 51 - protein, 6 aa, synthetic construct
  REGION 1..6: note = source = /note="Description of Artificial Sequence: Synthetic peptide"
QGISSW

SEQ ID NO: 52 - protein, 11 aa, synthetic construct [sequence omitted]

SEQ ID NO: 53 - protein, 9 aa, synthetic construct
  REGION 1..9: note = source = /note="Description of Artificial Sequence: Synthetic peptide"
GDSVSSNSA

SEQ ID NO: 54 - protein, 7 aa, synthetic construct
  REGION 1..7: note = source = /note="Description of Artificial Sequence: Synthetic peptide"
TYYRSKW

SEQ ID NO: 55 - protein, 13 aa, synthetic construct
  REGION 1..13: note = source = /note="Description of Artificial Sequence: Synthetic peptide"
CARDDDWNDFDYW

SEQ ID NO: 56 - protein, 10 aa, synthetic construct
  REGION 1..10: note = source = /note="Description of Artificial Sequence: Synthetic peptide"
GDSISSSSYY

SEQ ID NO: 57 - protein, 7 aa, synthetic construct
  REGION 1..7: note = source = /note="Description of Artificial Sequence: Synthetic peptide"
IFYSGNT

SEQ ID NO: 58 - protein, 18 aa, synthetic construct
  REGION 1..18: note = source = /note="Description of Artificial Sequence: Synthetic peptide"
CAREDDFLTDYYGAFDIW

SEQ ID NO: 59 - protein, 6 aa, synthetic construct
  REGION 1..6: note = source = /note="Description of Artificial Sequence: Synthetic peptide"
QSISSW

SEQ ID NO: 60 - skipped

SEQ ID NO: 61 - protein, 10 aa, synthetic construct [sequence omitted]

SEQ ID NO: 62 - protein, 8 aa, synthetic construct
  REGION 1..8: note = source = /note="Description of Artificial Sequence: Synthetic peptide"
GFDFRSYA

SEQ ID NO: 63 - protein, 10 aa, synthetic construct
  REGION 1..10: note = source = /note="Description of Artificial Sequence: Synthetic peptide"
INGEVGGSNT

SEQ ID NO: 64 - protein, 18 aa, synthetic construct
  REGION 1..18: note = source = /note="Description of Artificial Sequence: Synthetic peptide"
CVKGAGACGICNDDIDAW

SEQ ID NO: 65 - protein, 7 aa, synthetic construct
  REGION 1..7: note = source = /note="Description of Artificial Sequence: Synthetic peptide"
GSYAGSY

SEQ ID NO: 66 - skipped

SEQ ID NO: 67 - protein, 13 aa, synthetic construct
  REGION 1..13: note = source = /note="Description of Artificial Sequence: Synthetic peptide"
CGSTNDNDDGGLF

SEQ ID NO: 68 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 69 - protein, 533 aa, Macaca fascicularis
  VARIANT 74: note = X is any amino acid
MWEAQFLGLLFLQPLWVAPVKPPQPGAEISVVWAQEGAPAQLPCSPTIPLQDLSLLRRAGVTWQHQPDSGPPAXAPGHPPVPGHRPAAPYSWGPRPRRYTVLSVGPGGLRSGRLPLQPRVQLDERGRQRGDFSLWLRPARRADAGEYRATVHLRDRALSCRLRLRVGQASMTASPPGSLRTSDWVILNCSFSRPDRPASVHWFRSRGQGRVPVQGSPHHHLAESFLFLPHVGPMDSGLWGCILTYRDGFNVSIMYNLTVLGLEPATPLTVYAGAGSRVELPCRLPPAVGTQSFLTAKWAPPGGGPDLLVAGDNGDFTLRLEDVSQAQAGTYICHIRLQGQQLNATVTLAIITVTPKSFGSPGSLGKLLCEVTPASGQEHFVWSPLNTPSQRSFSGPWLEAQEAQLLSQPWQCQLHQGERLLGAAVYFTELSSPGAQRSGRAPGALRAGHLPLFLILGVLFLLLLVTGAFGFHLWRRQWRPRRFSALEQGIHPPQAQSKIEELEQEPELEPEPELERELGPEPEPGPEPEPEQL

SEQ ID NO: 70 - protein, 525 aa, Rattus norvegicus
MRQDLFLDLLLLQLLWEAPVVSSGPGKELSVVWAQEGAPVHLPCSLEFPHLDPNFLRRGWVTWQHRPDSDQPASIPALDLLQGMPSTRRHPPHRYTVLSVAPGGLRSGRQPLLSHVQLEKRGPQRGDFSLWLRPATRKDAGEYHAFVRLPDRDFSCSLRLRVGQASMIASPPGTLKPSDWVILNCSFSRPDRPVSVHWFQGQSRVPVHNSPRHYLAESFLLLPQVSPLDSGTWGCVLTYRDGFNVSITYNLKVQGLEPVAPLTVYAAEGSRVELPCHLPPVVGTPSLLIAKWTPPGGGPELPVTGKSGNFTLQLENVGRAQAGTYTCSIHLQGRQLSAAVTLAVITVTPKSFGLPGSPQKLLCEVVPASGEGRFVWRPLSDLSRSSLGPVLELQEAKLLAEQWQCQLYEGQKLLGATVYTAESSSGAWSAKRISGDLKGGHLFLSLILGALALFLLVTGAFGFHLWRRQLLRRRFSALEHGIRPPPVQSKIEELEREPETEMEPETEPDPEPQPEPELEPESRQL

SEQ ID NO: 71 - protein, 476 aa, unidentified
  REGION 1..476: note = Canis familiaris
MWEVQFLVLLLLQLLWVAPVAAPGSGTEVQVVWAQEGAPVQLPCSPTIPLQDVSLLRNAGVTWYHLPESGPAAPALSLRPAAPSARGPGPRSYVVLMRAPGGLRSGLVPTVNALALNSPGPTRFSLALQTVISLPHSWLFPGPSSLPGQPLASCWPSPHVGVGCVCEHPPFIHLTLLPARQSPLLFLLPSLQPQCSASLYLSLSASTLASSFPGLEPSGPLTVYTGAGSRVGLPCRLPPGVGTQSFLTAKWTPPGGGPDLLVAGDDGNFTLQLEVVNQAQAGTYTCHIHLQGQQLSTTVTLAVITVTPKSSGLPGNLRKLLCEVTPASGQERFVWSPLDKQSWRGSPGPCLEMQETRLLSQPWQCHVYQAERLLGTAVYLIDPAGPGAQRSGRAQGVLKTGHLSLLLILGILFLLLLMTGAFGFQLWRRQWRPRRFSALELGTHPPQAQSKIGELEQEPELELEPEPELEPEPEPE

SEQ ID NO: 72 - protein, 521 aa, Mus musculus
MREDLLLGFLLLGLLWEAPVVSSGPGKELPVVWAQEGAPVHLPCSLKSPNLDPNFLRRGGVIWQHQPDSGQPTPIPALDLHQGMPSPRQPAPGRYTVLSVAPGGLRSGRQPLHPHVQLEERGLQRGDFSLWLRPALRTDAGEYHATVRLPNRALSCSLRLRVGQASMIASPSGVLKLSDWVLLNCSFSRPDRPVSVHWFQGQNRVPVYNSPRHFLAETFLLLPQVSPLDSGTWGCVLTYRDGFNVSITYNLKVLGLEPVAPLTVYAAEGSRVELPCHLPPGVGTPSLLIAKWTPPGGGPELPVAGKSGNFTLHLEAVGLAQAGTYTCSIHLQGQQLNATVTLAVITVTPKSFGLPGSRGKLLCEVTPASGKERFVWRPLNNLSRSCPGPVLEIQEARLLAERWQCQLYEGQRLLGATVYAAESSSGAHSARRISGDLKGGHLVLVLILGALSLFLLVAGAFGFHWWRKQLLLRRFSALEHGIQPFPAQRKIEELERELETEMGQEPEPEPEPQLEPEPRQL

SEQ ID NO: 73 - protein, 6 aa, unidentified
  REGION 1..6: note = source = /note="Description of Unknown: LAG-3 motif"
KIEELE

SEQ ID NO: 74 - protein, 15 aa, synthetic construct
  REGION 1..15: note = source = /note="Description of Artificial Sequence: Synthetic peptide"
GGGGSGGGGSGGGGS

(19) United States
(12) Patent Application Publication
(10) Pub. No.: US 2024/0052032 A1
(43) Pub. Date: Feb. 15, 2024

(54) ANTI-LAG-3 ANTIBODIES AND COMPOSITIONS

(71) Applicant: Chia Tai Tianqing Pharmaceutical Group Co., Ltd., Lianyungang City (CN)

(72) Inventors: Michael Monrad GRANDAL, Ballerup (DK); Vikram Kjøller BHATIA, Charlottenlund (DK); Torben GJETTING, Jyllinge (DK); Camilla FRÖHLICH, København ø (DK); Gunther Roland GALLER, Jyllinge (DK); Michael KRAGH, Copenhagen N (DK); Ivan David HORAK, West Orange, NJ (US); Thomas BOUQUIN, Alleroed (DK); Mikkel Wandahl PEDERSEN, Alleroed (DK)

(21) Appl. No.: 18/490,500

(22) Filed: Oct. 19, 2023

Related U.S. Application Data
(63) Continuation of application No. 17/834,497, filed on Jun. 7, 2022, now Pat. No. 11,834,503.
Application No. 17/834,497 is a division of application No. 16/340,855, filed on Apr. 10, 2019, now Pat. No. 11,390,676.
(60) Provisional application No. 62/407,678, filed on Oct. 13, 2016.

(51) Int. Cl. C07K 16/28; A61P 37/04; A61P 35/00; A61K 9/00; C12N 15/85

(52) U.S. Cl. CPC C07K 16/2803; A61P 37/04; A61P 35/00; A61K 9/0019; C12N 15/85

(57) ABSTRACT

This invention relates to anti-LAG-3 antibodies and methods of using them in treating diseases and conditions that benefit from modulating LAG-3 activity, such as cancer.

### CROSS-REFERENCE TO RELATED APPLICATIONS

This application is a continuation of U.S. patent application Ser. No. 16/340,855, filed on Apr. 10, 2019, which is is a national stage application under 35 U.S.C. § 371 of International Patent Application PCT/EP2017/076188, filed on Oct. 13, 2017, which claims priority from U.S. Provisional Patent Application 62/407,678, filed on Oct. 13. 2016. The disclosures of those applications are incorporated by reference herein in their entirety.

### SEQUENCE LISTING

The instant application contains a Sequence Listing which has been submitted electronically in ASCII format and is hereby incorporated by reference in its entirety. The electronic copy of the Sequence Listing, created on Oct. 5, 2017, is named 022675_WO057_SL.txt and is 64.693 bytes in size.

### BACKGROUND OF THE INVENTION

LAG-3 (lymphocyte-activation gene 3), also known as CD223, is an immunoglobulin superfamily protein that functions as an immune checkpoint receptor. The mature protein is a 503-amino acid type I transmembrane protein with four extracellular Ig-like domains. It is expressed on various types of cells including activated T cells. T regulatory (Treg) cells, natural killer cells, B cells and plasmacytoid dendritic cells. Information on sequence data, exon/intron organization, and the chromosomal localization of LAG-3 indicates that it is closely related to CD4. Similar to CD4, LAG-3 binds MHC class II molecules, although with a higher affinity and at a distinct site compared to CD4.

LAG-3 is a co-inhibitory receptor that is thought to regulate T cell proliferation, activation and homeostasis in a manner similar to CTLA-4 and PD-1. Upon ligand binding to the extracellular domain, LAG-3 exerts its effect through subsequent signaling via the cytoplasmic domain. The best characterized ligand for LAG-3 is MHC class II (MHCII), but other LAG-3 ligands have been described, including LSECtin.

LAG-3 has no classical ITIM or ITSM motifs, but has a conserved KIEELE motif (SEQ ID NO: 73) which is thought to be indispensable for accomplishing its inhibitory effect on T-cell activity. The exact mechanism by which LAG-3 affects T-cell activity is poorly understood. LAG-3 inhibits T cell expansion by blocking entry of activated T-cells into the growth phase of the cell cycle, resulting in the accumulation of cells in the S-phase. LAG-3 is also thought to play a role in enhancing the suppressive activity of regulatory T-cells and in modulating dendritic cell function, Cancer cells have the ability to upregulate expression of MHCII, which binds LAG-3 on effector T-cells. thus inhibiting their activity and inducing tumor immune escape.

In view of the critical role of LAG-3 as an immune modulator, there is a need for new and improved immune therapies that target LAG-3 to treat cancers and certain disorders of the immune system.

### SUMMARY OF THE INVENTION

The present invention is directed to novel recombinant antibodies targeting LAG-3, as well as pharmaceutical compositions comprising one or more of these antibodies, and use of the antibodies and pharmaceutical compositions for enhancing immunity in a patient, and for treatment of cancers originating from tissues such as skin, lung, intestine, colon, ovary, brain, prostate, kidney, soft tissues, the hematopoietic system, head and neck, liver, bladder, breast, stomach, uterus and pancreas. Compared to currently available treatments for such cancers, including antibody treatments, it is contemplated that the antibodies of the invention may provide a superior clinical response either alone or in combination with another cancer therapeutic, such as an antibody targeting another immune checkpoint protein.

In one embodiment, the present invention provides an anti-LAG-3 antibody or an antigen-binding portion thereof, wherein the anti-LAG-3 antibody is any of the antibodies referred to herein as antibody 15646, 15532, 15723, 15595, 15431, 15572 or 15011, or a variant thereof, where the variant may, e.g., be of a different isotype or isotype subclass and/or contain certain minimum amino acid changes relative to antibody 15646, 15532, 15723, 15595, 15431, 15572 or 15011 without losing the antigen-binding specificity of the parent antibody.

In one embodiment, the anti-LAG-3 antibody competes for binding to human LAG-3 with an antibody whose heavy chain (H) CDR1-3 and light chain (L) CDR1-3 are the same as or derived from the H-CDR1-3 and L-CDR1-3 of antibody 15646, 15532, 15723, 15595, 15431, 15572 or 15011.

In one embodiment, the anti-LAG-3 antibody binds to the same epitope of human LAG-3 as an antibody whose heavy chain (H) CDR1-3 and light chain (L) CDR1-3 are the same as or derived from the H-CDR1-3 and L-CDR1-3 of antibody 15646, 15532, 15723, 15595, 15431, 15572 or 15011.

In one embodiment, the anti-LAG-3 antibody comprises an H-CDR3 comprising the H-CDR3 amino acid sequence of antibody 15646, 15532, 15723, 15595, 15431, 15572 or 15011.

In one embodiment, the anti-LAG-3 antibody comprises H-CDR1-3 comprising the H-CDR 1-3 sequences, respectively, of antibody 15646, 15532, 15723, 15595, 15431, 15572 or 15011.

In one embodiment, the anti-LAG-3 antibody comprises an L-CDR3 comprising the L-CDR3 amino acid sequence of antibody 15646, 15532, 15723, 15595, 15431, 15572 or 15011.

In one embodiment, the anti-LAG-3 antibody comprises L-CDR1-3 comprising the L-CDR1-3 sequences, respectively, of antibody 15646, 15532, 15723, 15595, 15431. 15572 or 15011.

In one embodiment, the anti-LAG-3 antibody comprises the H-CDR3 and L-CDR3 amino acid sequences of antibody 15646, 15532, 15723, 15595, 15431, 15572 or 15011.

In one embodiment, the anti-LAG-3 antibody comprises the H-CDR1-3 and L-CDR1-3 amino acid sequences of antibody 15646, 15532, 15723, 15595, 15431, 15572 or 15011.

In one embodiment, the anti-LAG-3 antibody competes for binding to human LAG-3 with antibody 15646, 15532, 15723, 15595, 15431, 15572 or 15011.

In one embodiment, the anti-LAG-3 antibody binds to the same epitope of human LAG-3 as antibody 15646, 15532, 15723, 15595, 15431, 15572 or 15011.

In one embodiment, the anti-LAG-3 antibody comprises a heavy chain complementarity-determining region (H-CDR) 3 comprising the amino acid sequence of SEQ ID NO: 37, 43, 46, 50, 55, 58, or 64.

In one embodiment, the anti-LAG-3 antibody compases H-CDR1-3 comprising the amino acid sequences, respectively, of SEQ ID NOs: 35-37; 41-43; 35, 42, and 46; 48-50: 53-55; 56-58; or 62-64

In one embodiment, the anti-LAG-3 antibody has a heavy chain variable domain (VH) that is at least 90% (e.g., at least 92%, at least 95%, at least 98%, or at least 99%) identical in sequence to the VH amino acid sequence of SEQ ID NO: 3, 7, 11, 15, 19, 23 or 27.

In one embodiment. the anti-LAG-3 antibody has a VH that comprises SEQ ID NO: 3, 7, 11, 15, 19, 23 or 27.

In one embodiment, the anti-LAG-3 antibody has a heavy chain (HC) that comprises the VH amino acid sequence of SEQ ID NO: 3, 7, 11, 15, 19, 23 or 27 and the heavy chain constant region amino acid sequence of SEQ ID NO: 30.

In one embodiment, the anti-LAG-3 antibody comprises a light chain complementarity-determining region (L-CDR) 3 comprising the amino acid sequence of SEQ ID NO: 40, 52, 61, or 67.

In one embodiment, the anti-LAG-3 antibody comprises L-CDR1-3 comprising the amino acid sequences, respectively, of SEQ ID NOs: 38-40: 44, 45, and 40; 44, 47, and 40: 51, 47, and 52; 59-61; or 65-67.

In one embodiment, the anti-LAG-3 antibody has a light chain variable domain (VL) that is at least 90% (e.g., at least 92%, at least 95%, at least 98%, or at least 99%) identical in sequence to the VL amino acid sequence of SEQ ID NO: 4, 8, 12, 16, 20, 24 or 28.

In one embodiment, the anti-LAG-3 antibody has a VL that comprises SEQ ID NO: 4, 8, 12, 16, 20, 24 or 28.

In one embodiment, the anti-LAG-3 antibody has a light chain (LC) that comprises the VL amino acid sequence of SEQ ID NO: 4, 8, 12, 16, 20, 24 or 28 and the light chain constant region amino acid sequence of SEQ ID NO: 32 or 34.

In one embodiment, the anti-LAG-3 antibody comprises the H-CDR3 and L-CDR3 amino acid sequences of SEQ ID NOs: 37 and 40; 43 and 40; 46 and 40; 50 and 52; 55 and 40; 58 and 61; or 64 and 67; respectively.

In certain embodiments, the anti-LAG-3 antibody or antigen-binding portion of the invention comprises the H-CDR1-3 and L-CDR1-3 amino acid sequences of:

a) SEQ ID NOs: 35, 36, 37, 38, 39, and 40, respectively;
b) SEQ ID NOs: 41, 42, 43, 44, 45, and 40, respectively;
c) SEQ ID NOs: 35, 42, 46, 44, 47, and 40, respectively;
d) SEQ ID NOs: 48, 49, 50, 51, 47, and 52, respectively;
e) SEQ ID NOs: 53, 54, 55, 44, 45, and 40, respectively;
f) SEQ ID NOs: 56, 57, 58, 59, 60, and 61, respectively; or
g) SEQ ID NOs: 62, 63, 64, 65, 66, and 67, respectively.

In certain embodiments, the anti-LAG-3 antibody or antigen-binding portion thereof of the invention:

a) has a VH that is at least 90% (e.g. at least 92%, at least 95%, at least 98%, or at least 99%) identical in sequence to the amino acid sequence of SEQ ID NO: 3, and a VL that is at least 90% (e.g. at least 92%, at least 95%, at least 98%. or at least 99%) identical in sequence to the amino acid sequence of SEQ ID NO: 4;
b) has a VH that is at least 90% (e.g. at least 92%, at least 95%, at least 98%, or at least 99%) identical in sequence to the amino acid sequence of SEQ ID NO: 7, and a VL that is at least 90% (e.g. at least 92%, at least 95%, at least 98%, or at least 99%) identical in sequence to the amino acid sequence of SEQ ID NO: 8;
c) has a VH that is at least 90% (e.g. at least 92%, at least 95%, at least 98%, or at least 99%) identical in sequence to the amino acid sequence of SEQ ID NO: 11, and a VL that is at least 90% (e.g. at least 92%, at least 95%, at least 98%, or at least 99%) identical in sequence to the amino acid sequence of SEQ ID NO: 12;
d) has a VH that is at least 90% (e.g. at least 92%, at least 95%, at least 98%, or at least 99%) identical in sequence to the amino acid sequence of SEQ ID NO: 15, and a VL that is at least 90% (e.g. at least 92%, at least 95%, at least 98%, or at least 99%) identical in sequence to the amino acid sequence of SEQ ID NO: 16;
e) has a VH that is at least 90% (e.g. at least 92%, at least 95%, at least 98%, or at least 99%) identical in sequence to the amino acid sequence of SEQ ID NO: 19, and a VL that is at least 90% (e.g. at least 92%, at least 95%, at least 98%, or at least 99%) identical in sequence to the amino acid sequence of SEQ ID NO: 20;
f) has a VH that is at least 90% (e.g. at least 92%, at least 95%, at least 98%, or at least 99%) identical in sequence to the amino acid sequence of SEQ ID NO: 23, and a VL that is at least 90% (e.g. at least 92%, at least 95%, at least 98%, or at least 99%) identical in sequence to the amino acid sequence of SEQ ID NO: 24; or
g) has a VH that is at least 90% (e.g. at least 92%, at least 95%, at least 98%, or at least 99%) identical in sequence to the amino acid sequence of SEQ ID NO: 27, and a VL that is at least 90% (e.g. at least 92%, at least 95%, at least 98%, or at least 99%) identical in sequence to the amino acid sequence of SEQ ID NO: 28.

In certain embodiments, the anti-LAG-3 antibody or antigen-binding portion of the invention:

a) has a VH that comprises or consists of the amino acid sequence of SEQ ID NO: 3, and a VL that comprises or consists of the amino acid sequence of SEQ ID NO: 4;
b) has a VH that comprises or consists of the amino acid sequence of SEQ ID NO: 7, and a VL that comprises or consists of the amino acid sequence of SEQ ID NO: 8;
c) has a VH that comprises or consists of the amino acid sequence of SEQ ID NO: 11, and a VL that comprises or consists of the amino acid sequence of SEQ ID NO: 12;
d) has a VH that comprises or consists of the amino acid sequence of SEQ ID NO: 15, and a VL that comprises or consists of the amino acid sequence of SEQ ID NO: 16;
e) has a VH that comprises or consists of the amino acid sequence of SEQ ID NO: 19, and a VL that comprises or consists of the amino acid sequence of SEQ ID NO: 20;
f) has a VH that comprises or consists of the amino acid sequence of SEQ ID NO: 23, and a VL that comprises or consists of the amino acid sequence of SEQ ID NO: 24; or
g) has a VH that comprises or consists of the amino acid sequence of SEQ ID NO: 27, and a VL that comprises or consists of the amino acid sequence of SEQ ID NO: 28.

In certain embodiments, the anti-LAG-3 antibody:

a) has an LC that comprises or consists of the amino acid sequences of SEQ ID NOs: 4 and 34; and an HC that comprises or consists of the amino acid sequences of SEQ ID NOs: 3 and 30;
b) has an LC that comprises or consists of the amino acid sequences of SEQ ID NOs: 8 and 34; and an HC that comprises or consists of the amino acid sequences of SEQ ID NOs: 7 and 30;
c) has an LC that comprises or consists of the amino acid sequences of SEQ ID NOs: 12 and 34; and an HC that comprises or consists of the amino acid sequences of SEQ ID NOs: 11 and 30;
d) has an LC that comprises or consists of the amino acid sequences of SEQ ID NOs: 16 and 34; and an HC that comprises or consists of the amino acid sequences of SEQ ID NOs: 15 and 30;
e) has an LC that comprises or consists of the amino acid sequences of SEQ ID NOs: 20 and 34; and an HC that comprises or consists of the amino acid sequences of SEQ ID NOs: 19 and 30;
f) has an LC that comprises or consists of the amino acid sequences of SEQ ID NOs: 24 and 34; and an HC that comprises or consists of the amino acid sequences of SEQ ID NOs: 23 and 30; or
g) has an LC that comprises or consists of the amino acid sequences of SEQ ID NOs: 28 and 32; and an HC that comprises or consists of the amino acid sequences of SEQ ID NOs: 27 and 30.

In some embodiments, the anti-LAG-3 antibody or antigen-binding portion of the invention has at least one of the following properties:

a) at a concentration of 20 μg/mL, reduces the binding of human LAG-3 to human MHC class II on A375 cells by greater than 85% compared to a negative control antibody as determined by a flow cytometric competition assay;
b) at a concentration of 20 μg/mL, reduces the binding of human LAG-3 to human MHC class II on A375 cells to between 35% and 85% compared to a negative control antibody as determined by a flow cytometric competition assay:
c) blocks binding between human LAG-3 expressed on Jurkat cells and human MHC class II expressed on Raji cells;
d) binds to human LAG-3 with an EC₅₀ of 0.1 nM or less as measured by flow cytometry;
e) binds to cynomolgus LAG-3 with an EC₅₀ of 0.3 nM or less as measured by flow cytometry;
f) binds to human LAG-3 with a K_{D} of 3.0×10⁻⁸ or less as measured by surface plasmon resonance;
g) binds to cynomolgus LAG-3 with a K_{D} of 1.5×10⁻⁷ or less as measured by surface plasmon resonance;
h) binds to mouse LAG-3 with a K_{D} of 3.5×10⁻⁸ or less as measured by surface plasmon resonance;
i) stimulates IL-2 production in Staphylococcal enterotoxin B (SEB) treated human peripheral blood mononuclear cells (PBMCs);
j) reduces cellular levels of LAG-3 in human T cells;
k) reduces soluble levels of LAG-3 in the culture of human T cells;
l) induces tumor growth regression in vivo;
m) delays tumor growth in vivo; and
n) does not bind to the same epitope of human LAG-3 as antibody 25F7-Lag3.5.

Examples of such an antibody include, without limitation, antibody 15646 (having at least properties b, c, d, e, i, and n), antibody 15532 (having at least properties a, c, d, e, f, g, i, j, k, m, and n), antibody 15723 (having at least properties b, c, d, e, i, and n), antibody 15595 (having at least properties a, c, d, e, i, and n), antibody 15431 (having at least properties a, c, d, e, f, g. i, and n), antibody 15572 (having at least properties b, c, d. e, f, g, i, and n), and antibody 15011 (having at least properties a, c, d, e, f, g, h, i, j. k. I, m, and n). In some embodiments, the anti-TIM-3 antibody or antigen-binding portion of the invention has at least 13, 12, 11, 10, 9, 8, 7, 6, 5, 4, 3, 2, or 1 of said properties. In some embodiments. the anti-TIM-3 antibody or antigen-binding portion of the invention has at least properties b, c, d, e, i, and n; at least properties a, c, d, e, f, g, i, j, k, m. and n; at least properties a, c, d, e, i, and n; at least properties a, c, d, e, f, g, i, and n; at least properties b, c, d, e, f, g, i, and n; or at least properties a, c, d, e, f, g, h, i, j, k, l, m, and n.

In some embodiments, the anti-LAG-3 antibody or antigen-binding portion of the invention competes for binding to human LAG-3 with antibody 15011, 15572, and/or 15431.

In some embodiments, the anti-LAG-3 antibody or antigen-binding portion of the invention binds to an epitope of human LAG-3 having:

a) amino acid residues H85, P86, A87, P89, S91, W92, and G93 of SEQ ID NO: 68:
b) amino acid residues A40, Q41, P43, P46, P49, D52, T62, Q64, H65, Q66, P67, D68, G93, P94, P96, R98, Y99, T100, V101, P106, G107, R119, E124, R129, G130, D131, S133, R137, P138, D143, R148, and R163 of SEQ ID NO: 68;
c) amino acid residues A40, Q41, P43, P46, P49, D52, T62, Q64, H65, Q66, P67, D68, P96, Y99, T100, V101, P106, G107, R119, E124, R129, G130, D131, S133, R137, P138, D143, R148, and R163 of SEQ ID NO: 68; or
d) amino acid residues G107, L109, R110, and S111 of SEQ ID NO. 68.

In some embodiments, the anti-LAG-3 antibody or antigen-binding portion of the invention binds to an epitope having amino acid residues 98-105 of SEQ ID NO: 68. Examples of such an antibody include, without limitation, antibodies 15532, 15431, 15572, and 15011.

In some embodiments, the anti-LAG-3 antibody or antigen-binding portion of the invention binds to an epitope having:

a) amino acid residues 78-105 and 123-131 of SEQ ID NO: 68:
b) amino acid residues 23-30, 40-66, 88-105, 123-137, and 148-152 of SEQ ID NO: 68; or
c) amino acid residues 23-30, 40-66, 98-105, 118-137, and 148-161 of SEQ ID NO: 68

In some embodiments, the antibody of the present invention is of isotype IgG, for example, of isotype IgG subclass IgG₁ or IgG₂. In certain embodiments, the antibody comprises at least one mutation in the F_{c} region. In particular embodiments, the antibody comprises a mutation in one or more of heavy chain amino acid positions 228, 234 and 235, which are numbered according to the IMGT® numbering scheme. For example, one or both of the amino acid residues at positions 234 and 235 may be mutated from Leu to Ala, and/or the amino acid residue at position 228 may be mutated from Ser to Pro.

In another aspect, the present invention provides pharmaceutical compositions comprising at least one (e.g., one) anti-LAG-3 antibody or antigen-binding portion thereof as described herein and a pharmaceutically acceptable excipient, optionally with an additional therapeutic, such as an anti-cancer antibody therapeutic.

The present invention further provides isolated nucleic acid molecules comprising a nucleotide sequence that encodes the heavy chain or an antigen-binding portion thereof, a nucleotide sequence that encodes the light chain or an antigen-binding portion thereof, or both, of an anti-LAG-3 antibody as described herein. The invention also provides vectors comprising such an isolated nucleic acid molecule, wherein said vector may further comprise an expression control sequence.

The present invention also provide host cells comprising a nucleotide sequence that encodes the heavy chain or an antigen-binding portion thereof, a nucleotide sequence that encodes the light chain or an antigen-binding portion thereof, or both, of an anti-LAG-3 antibody as described herein.

The present invention also provides a method for producing an antibody or antigen-binding portion thereof as described herein, comprising providing a host cell that comprises a nucleotide sequence that encodes the heavy chain or an antigen-binding portion thereof and a nucleotide sequence that encodes the light chain or an antigen-binding portion thereof of an anti-LAG-3 antibody as described herein, culturing said host cell under conditions suitable for expression of the antibody or portion, and isolating the resulting antibody or portion.

The present invention also provides a multi-specific (e.g., bi-specific) binding molecule comprising the antigen-binding portion of an anti-LAG-3 antibody described herein and the antigen-binding portion of another, distinct antibody such as another anti-LAG-3 antibody (e.g., as described herein) or an antibody that targets a different protein, such as another immune checkpoint protein, a cancer antigen, or another cell surface molecule whose activity mediates a disease condition such as cancer.

The present invention also provides a method for treating a patient with a LAG-3-related disorder, comprising administering to said patient an anti-LAG-3 antibody or an antigen-binding portion thereof, a pharmaceutical composition, or a bi-specific binding molecule as described herein. Unless otherwise indicated, a patient refers herein to a human patient

The present invention also provides a method for enhancing immunity in a patient, comprising administering to said patient an anti-LAG-3 antibody or an antigen-binding portion thereof, a pharmaceutical composition, or a bi-specific binding molecule as described herein.

The present invention further provides a method for treating cancer in a patient, comprising administering to said patient an anti-LAG-3 antibody or an antigen-binding portion thereof, a pharmaceutical composition, or a bi-specific binding molecule as described herein. In some embodiments, the cancer originates in a tissue selected from skin, lung, intestine, colon, ovary, brain, prostate, kidney, soft tissues, hematopoietic system, head and neck, liver, bladder, breast, stomach, uterus and pancreas. In certain embodiments, the cancer is fibrosarcoma, non-small cell lung cancer, melanoma, glioblastoma, gliosarcoma, or colorectal cancer.

Any of the above methods may further comprise administration of, e.g., a chemotherapeutic agent, an anti-neoplastic agent, an anti-angiogenic agent. a tyrosine kinase inhibitor, a LAG-3 pathway inhibitor, or radiation therapy. In some embodiments, the method further comprises administration of retinoic acid, phenylbutyrate, all-trans-retinoic acid, and/or active form vitamin D.

The present invention further provides the use of an antibody composition comprising an anti-LAG-3 antibody or antigen-binding portion as described herein for the manufacture of a medicament for treating a patient with a LAG-3-related disorder, treating cancer in a patient, and/or enhancing immunity in a patient in need thereof.

The present invention further provides an anti-LAG-3 antibody or antigen-binding portion as described herein for treating a patient with a LAG-3-related disorder, treating cancer in a patient, and/or enhancing immunity in a patient in need thereof.

The present invention further provides an article of manufacture comprising an anti-LAG-3 antibody or antigen-binding portion as described herein, wherein said article of manufacture is suitable for treating a patient with a LAG-3-related disorder, treating cancer in a patient, and/or enhancing immunity in a patient in need thereof.

### BRIEF DESCRIPTION OF THE DRAWINGS

FIGS. 1A-1C show representative flow cytometry dot plots for antibodies generated as described in Example 2. FIG. 1A: an antibody clone binding specifically to human LAG-3-transfected cells FIG. 1B: an antibody clone binding non-specifically to CHO-S cells. FIG. 1C: an antibody clone that does not bind to any of the cell populations used in the screening.

FIG. 2 shows the level of IL-2 production from SEB-stimulated PBMCs after treatment with anti-LAG-3 monoclonal antibodies.

FIG. 3 shows the level of IL-2 production from SEB-stimulated PBMCs after treatment with anti-LAG-3 monoclonal antibodies. Each dot represents the level of IL-2 secretion from a single PBMC donor.

FIG. 4 shows the level of luminescence in a LAG-3-MHC class II blocking assay after treatment with anti-LAG-3 monoclonal antibodies. Data are presented as means+SEM.

FIG. 5 shows the levels of LAG-3 in lysates and supernatants from a LAG-3 transfected T cell line after treatment with anti-LAG-3 monoclonal antibodies. Data are presented as means +SEM. Asterisks indicate a statistically significant difference between 15532 and reference antibody analogues (*: p<0.05. ***: p<0.001, ****: p<0.0001).

FIG. 6 demonstrates the in vivo efficacy of antibody 15011 in two syngeneic murine tumor models.

FIG. 7 demonstrates the in vivo efficacy of antibody 15532 in a semi-humanized xenograft tumor model, where the immune system in NOG mice was reconstituted with human PBMC and the mice engrafted with human melanoma A375 cells.

FIG. 8 shows an overview of the epitope groups (epitope bins) identified by binding competition analysis of a panel of thirteen anti-LAG-3 antibodies. Antibodies connected by black lines indicate cross blocking activity in both orientations (ligand and analyte). Antibodies connected by dashed lines indicate unidirectional blocking where the antibody is blocked when tested as an analyte only. Antibodies are grouped according to competition patterns with other anti-LAG-3 antibodies.

FIG. 9 shows a cluster analysis of the epitope diversity of ligand antibodies. Antibodies with branching points at x-axis values above 0 have epitopes that are distinct based on the competition for LAG-3 binding of other antibodies in the panel.

### DETAILED DESCRIPTION OF THE INVENTION

The present invention provides new anti-human LAG-3 antibodies that can be used to enhance the immune system in a human patient, such as a cancer patient. Unless otherwise stated, as used herein, “LAG-3” refers to human LAG-3. A human LAG-3 polypeptide sequence is available under UniProt Accession No. P18627 (LAG3_HUMAN) (SEQ ID NO: 68).

The term “antibody” (Ab) or “immunoglobulin” (Ig), as used herein, refers to a tetramer comprising two heavy (H) chains (about 50-70 kDa) and two light (L) chains (about 25 kDa) inter-connected by disulfide bonds. Each heavy chain is comprised of a heavy chain variable domain (VH) and a heavy chain constant region (CH). Each light chain is composed of a light chain variable domain (VL) and a light chain constant region (CL). The VH and VL domains can be subdivided further into regions of hypervariability, termed “complementarity determining regions” (CDRs), interspersed with regions that are more conserved, termed “framework regions” (FRs). Each VH and VL is composed of three CDRs (H-CDR herein designates a CDR from the heavy chain; and L-CDR herein designates a CDR from the light chain) and four FRs, arranged from amino-terminus to carboxyl-terminus in the following order. FR1, CDR1, FR2, CDR2, FR3, CDR3, FR4. The assignment of amino acid numbers in the heavy or light chain may be in accordance with IMGT® definitions (Lefranc et al., Dev Comp Immunol 27(1): 55-77 (2003)); or the definitions of Kabat, Sequences of Proteins of Immunological Interest (National Institutes of Health, Bethesda, MD (1987 and 1991)); Chothia & Lesk, J. Mol. Biol. 196: 901-917 (1987); or Chothia et al., Nature 342: 878-883 (1989).

The term “recombinant antibody” refers to an antibody that is expressed from a cell or cell line comprising the nucleotide sequence(s) that encode the antibody, wherein said nucleotide sequence(s) are not naturally associated with the cell.

The term “isolated protein”, “isolated polypeptide” or “isolated antibody” refers to a protein, polypeptide or antibody that by virtue of its origin or source of derivation (1) is not associated with naturally associated components that accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

As used herein, the term “germline” refers to the nucleotide and amino acid sequences of antibody genes and gene segments as they are passed from parents to offspring via germ cells. Germline sequences are distinguished from the nucleotide sequences encoding antibodies in mature B cells, which have been altered by recombination and hypermutation events during the course of B cell maturation. An antibody that “utilizes” a particular germline sequence has a nucleotide or amino acid sequence that aligns with that germline nucleotide sequence or with the amino acid sequence that it specifies more closely than with any other germline nucleotide or amino acid sequence.

The term “affinity” refers to a measure of the attraction between an antigen and an antibody. The intrinsic attractiveness of the antibody for the antigen is typically expressed as the binding affinity equilibrium constant (K_{D}) of a particular antibody-antigen interaction. An antibody is said to specifically bind to an antigen when the K_{D} is ≤1 mM, preferably ≤100 nM. A K_{D} binding affinity constant can be measured, e.g., by surface plasmon resonance (BIAcore™) or Bio-Layer Interferometry, for example using the IBIS MX96 SPR system from IBIS Technologies or the Octet™ system from ForteBio.

The term “k_{off}” refers to the dissociation rate constant of a particular antibody-antigen interaction. A k_{off} dissociation rate constant can be measured, e.g., by SPR (surface plasmon resonance), for example using the using the IBIS MX96 system.

The term “epitope” as used herein refers to a portion (determinant) of an antigen that specifically binds to an antibody or a related molecule such as a bi-specific binding molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest (e.g., LAG-3) or a relevant portion thereof, then screening for binding to the epitope.

One can determine whether an antibody binds to the same epitope as or competes for binding with an anti-LAG-3 antibody of the invention by using methods known in the art, including, without limitation, competition assays, epitope binning, and alanine scanning. In one embodiment. one allows the anti-LAG-3 antibody of the invention to bind to LAG-3 under saturating conditions, and then measures the ability of the test antibody to bind to LAG-3. If the test antibody is able to bind to LAG-3 at the same time as the reference anti-LAG-3 antibody, then the test antibody binds to a different epitope than the reference anti-LAG-3 antibody. However, if the test antibody is not able to bind to LAG-3 at the same time, then the test antibody binds to the same epitope, an overlapping epitope, or an epitope that is in close proximity to the epitope bound by the anti-LAG-3 antibody of the invention. This experiment can be performed using, e.g., ELISA, RIA, BIACORE™, SPR, Bio-Layer Interferometry or flow cytometry. To test whether an anti-LAG-3 antibody cross-competes with another anti-LAG-3 antibody, one may use the competition method described above in two directions. i.e., determining if the known antibody blocks the test antibody and vice versa. Such cross-competition experiments may be performed, e.g., using an IBIS MX96 SPR instrument or the Octet™ system.

An antibody that binds to the same epitope as or competes for binding with an antibody of the invention preferably has MHCII blocking activity, e.g. as determined using the flow cytometric competition assay described in Example 6. An antibody that binds to the same epitope as or competes for binding with an antibody of the invention may reduce binding by at least, e.g., 35%, 40%, 45%, 50%, 55%, 60%, 65%, 70%, 75%, or 80%, or preferably by at least 85%, 86%, 87%, 88%, 89%, 90%, 91%, 92%, 93%, 94%, 95%, 96%, 97%, 98%, 99%, or 100%.

The term “chimeric antibody” refers in its broadest sense to an antibody that contains one or more regions from one antibody and one or more regions from one or more other antibodies, typically an antibody that is partially of human origin and partially of non-human origin, i.e., derived in part from a non-human animal, for example a mouse, rat or other rodent, or an avian such as a chicken. Chimeric antibodies are preferred over non-human antibodies in order to reduce the risk of a human anti-antibody response, e.g., a human anti-mouse antibody response in the case of a murine antibody. An example of a typical chimeric antibody is one in which the variable domain sequences are murine while the constant region sequences are human. In the case of a chimeric antibody, the non-human parts may be subjected to further alteration in order to humanize the antibody. The chimeric antibodies described herein may have, e.g., chicken variable domain sequences and human constant region sequences.

The term “humanize” refers to the fact that where an antibody is wholly or partially of non-human origin (for example, a murine or chicken antibody obtained from immunization of mice or chickens, respectively, with an antigen of interest, or a chimeric antibody based on such a murine or chicken antibody), it is possible to replace certain amino acids, in particular in the framework regions and constant regions of the heavy and light chains, in order to avoid or minimize an immune response in humans. Although it is not possible to precisely predict the immunogenicity, and thereby the human anti-antibody response, of a particular antibody, non-human antibodies tend to be more immunogenic in humans than human antibodies. Chimeric antibodies, where the foreign (e.g. rodent or avian) constant regions have been replaced with sequences of human origin, have been shown to be generally less immunogenic than antibodies of fully foreign origin, and the trend in therapeutic antibodies is towards humanized or fully human antibodies. Chimeric antibodies or other antibodies of non-human origin thus can be humanized to reduce the risk of a human anti-antibody response.

For chimeric antibodies, humanization typically involves modification of the framework regions of the variable domain sequences. Amino acid residues that are part of complementarity determining regions (CDRs) most often will not be altered in connection with humanization, although in certain cases it may be desirable to alter individual CDR amino acid residues, for example to remove a glycosylation site, a deamidation site, an aspartate isomerization site or an undesired cysteine or methionine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, preferably by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in a CDR sequence, it may therefore be desirable to remove the site, typically by conservative substitution to remove one of the implicated residues.

Numerous methods for humanization of an antibody sequence are known in the art; see, e.g., the review by Almagro & Fransson, Front Biosci. 13: 1619-1633 (2008). One commonly used method is CDR grafting, which for, e.g., a murine-derived chimeric antibody involves identification of human germline gene counterparts to the murine variable domain genes and grafting of the murine CDR sequences into this framework. The specificity of an antibody's interaction with a target antigen resides primarily in the amino acid residues located in the six CDRs of the heavy and light chain. The amino acid sequences within CDRs are therefore much more variable between individual antibodies than sequences outside of CDRs. Because CDR sequences are responsible for most antibody-antigen interactions, it is possible to express recombinant antibodies that mimic the properties of a specific naturally occurring antibody, or more generally any specific antibody with a given amino acid sequence, e.g., by constructing expression vectors that express CDR sequences from the specific antibody grafted into framework sequences from a different antibody. As a result, it is possible to “humanize” a non-human antibody and still substantially maintain the binding specificity and affinity of the original antibody. CDR grafting may be based on the Kabat CDR definitions, although a more recent publication (Magdelaine-Beuzelin et al., Crit Rev. Oncol Hematol. 64: 210-225 (2007)) has suggested that the IMGT® definition (the international ImMunoGeneTics information system®) may improve the result of the humanization (see Lefranc et al., Dev. Comp Immunol. 27: 55-77 (2003)).

In some cases, CDR grafting may reduce the binding specificity and affinity, and thus the biological activity, of a CDR-grafted antibody as compared to the parent antibody from which the CDRs are obtained. Back mutations (sometimes referred to as “framework repair”) may be introduced at selected positions of the CDR-grafted antibody. typically in the framework regions, in order to reestablish the binding specificity and affinity of the parent antibody. Positions for possible back mutations can be identified using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing. in which non-surface exposed residues of non-human origin are retained, while surface residues are altered to human residues.

In certain cases, it also may be desirable to alter one or more CDR amino acid residues in order to improve binding affinity to the target epitope. This is known as “affinity maturation” and may optionally be performed in connection with humanization, for example in situations where humanization of an antibody leads to reduced binding specificity or affinity and it is not possible to sufficiently improve the binding specificity or affinity by back mutations alone. Various affinity maturation methods are known in the art, for example the in vitro scanning saturation mutagenesis method described by Burks et al., Proc Natl Acad Sci USA, 94: 412-417 (1997), and the stepwise in vitro affinity maturation method of Wu et al., Proc Natl Acad Sci USA 95: 6037-6042 (1998).

The term “antigen-binding portion” of an antibody (or simply “antibody portion”). as used herein, refers to one or more portions or fragments of an antibody that retain the ability to specifically bind to an antigen (e.g., human LAG-3, or a portion thereof). It has been shown that certain fragments of a full-length antibody can perform the antigen-binding function of the antibody. Examples of binding fragments encompassed within the term “antigen-binding portion” include (i) a Fab fragment: a monovalent fragment consisting of the V_{L}, V_{H}, C_{L} and C_{H}1 domains. (ii) a F(ab′)₂ fragment: a bivalent fragment comprising two Fab fragments linked by a disulfide bridge at the hinge region; (iii) an Fd fragment consisting of the V_{H} and C_{H}1 domains; (iv) a Fv fragment consisting of the V_{L} and V_{H} domains of a single arm of an antibody, (v) a dAb fragment, which consists of a V_{H} domain, and (vi) an isolated complementarity determining region (CDR) capable of specifically binding to an antigen. Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} domains pair to form monovalent molecules (known as single chain Fv (scFv)). Also within the invention are antigen-binding molecules comprising a V_{H} and/or a V_{L}. In the case of a V_{H}, the molecule may also comprise one or more of a CH1, hinge, CH2, or CH3 region. Such single chain antibodies are also intended to be encompassed within the term “antigen-binding portion” of an antibody. Other forms of single chain antibodies, such as diabodies, are also encompassed. Diabodies are bivalent, bi-specific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites.

Antibody portions, such as Fab and F(ab′)₂ fragments, can be prepared from whole antibodies using conventional techniques, such as papain or pepsin digestion of whole antibodies. Moreover, antibodies, antibody portions and immunoadhesion molecules can be obtained using standard recombinant DNA techniques, e.g., as described herein.

The class (isotype) and subclass of anti-LAG-3 antibodies may be determined by any method known in the art. In general, the class and subclass of an antibody may be determined using antibodies that are specific for a particular class and subclass of antibody. Such antibodies are available commercially. The class and subclass can be determined by ELISA, Western Blot as well as other techniques. Alternatively, the class and subclass may be determined by sequencing all or a portion of the constant regions of the heavy and/or light chains of the antibodies. comparing their amino acid sequences to the known amino acid sequences of various classes and subclasses of immunoglobulins. and determining the class and subclass of the antibodies.

Unless otherwise indicated, all antibody amino acid residue numbers referred to in this disclosure are those under the IMGT® numbering scheme.

### Anti-LAG-3 Antibodies

The present invention provides antibodies directed against LAG-3, and antigen-binding portions thereof. In a particular embodiment, the antibodies disclosed herein are human antibodies generated from transgenic rats that are able to generate antibodies with human idiotypes. In another embodiment, the antibodies are chicken-derived chimeric antibodies comprising chicken CDR sequences and human framework regions, where the framework regions have been subjected to humanization.

One advantage of the novel anti-LAG-3 antibodies of the invention is that they are able to enhance activity of T-cells as measured by increased IL-2 production; see, e.g., Example 7. While not wishing to be bound by any particular theory, it is believed that the anti-LAG-3 antibodies of the invention are able to block the interaction of LAG-3 with its putative ligands such as MHCII and LSECtin. The antibodies may accomplish this directly via blocking of the ligand binding region, as demonstrated, e.g., in Example 6, or via induction of LAG-3 internalization, which is contemplated as a possible mechanism of action behind the results presented in Example 9. Another potential advantage of the anti-LAG-3 antibodies of the invention is a low level of secondary effector functions in antibodies having the “LALA” mutations (L234A/L235A), which hinder significant antibody binding to human FcgR (Fc gamma receptors) and hence depletion of effector T-cells.

In one embodiment, the anti-LAG-3 antibody has a heavy chain CDR3 (H-CDR3) that is at least 90% identical in sequence to any one of SEQ ID NOs: 37, 43, 46, 50, 55. 58, and 64, e.g. at least 92% identical, such as at least 95%, 96%, at least 97%, at least 98% or at least 99% identical to any one of SEQ ID NOs: 37, 43, 46, 50, 55, 58, and 64.

In one embodiment, the anti-LAG-3 antibody has a heavy chain variable domain (VH) that is at least 90% identical in sequence to any one of SEQ ID NOs: 3, 7, 11, 15, 19, 23 or 27, e.g. at least 92% identical, such as at least 95%, 96%, at least 97%, at least 98% or at least 99% identical to any one of SEQ ID NOs: 3, 7, 11, 15, 19, 23 or 27.

In another embodiment, the anti-LAG-3 antibody has a heavy chain variable domain (VH) that is at least 90% identical in sequence to any one of SEQ ID NOs: 3, 7, 11, 15, 19, 23 or 27, e.g. at least 92% identical, such as at least 95%, at least 96%, at least 97%, at least 98% or at least 99% identical to any one of SEQ ID NOs: 3, 7, 11, 15, 19, 23 or 27; and a heavy chain constant region that is at least 90% identical in sequence to SEQ ID NO: 30, e.g. at least 92% identical, such as at least 95%, at least 96%, at least 97%, at least 98% or at least 99% identical to SEQ ID NO: 30.

In another embodiment, the anti-LAG-3 antibody has a heavy chain (HC) that comprises the VH amino acid sequence of any one of SEQ ID NOs: 3, 7, 11, 15, 19, 23 or 27 and the heavy chain constant region amino acid sequence of SEQ ID NO: 30.

In one embodiment, the anti-LAG-3 antibody has a light chain CDR3 (L-CDR3) that is at least 90% identical in sequence to any one of SEQ ID NOS: 40, 52, 61, and 67, e.g. at least 92% identical, such as at least 95%, 96%, at least 97%, at least 98% or at least 99% identical to any one of SEQ ID NOs: 40, 52, 61, and 67.

In another embodiment. the anti-LAG-3 antibody has a light chain variable domain (VL) that is at least 90% identical in sequence to the VL amino acid sequence of any one of SEQ ID NOs: 4, 8, 12, 16, 20, 24 or 28, e.g. at least 92% identical, such as at least 95%, at least 96%, at least 97%, at least 98% or at least 99% identical to any one of SEQ ID NOs: 4, 8, 12, 16, 20, 24 or 28.

In another embodiment, the anti-LAG-3 antibody has a light chain variable domain (VL) that is at least 90% identical in sequence to the VL amino acid sequence of any one of SEQ ID NOs: 4, 8, 12, 16, 20 or 24, e.g. at least 92% identical, such as at least 95%, at least 96%, at least 97%, at least 98% or at least 99% identical to any one of SEQ ID NOs: 4, 8, 12, 16, 20 or 24; and a light chain constant region amino acid sequence that is at least 90% identical in sequence to SEQ ID NO: 34, e.g. at least 92% identical, such as at least 95%, at least 96%, at least 97%, at least 98% or at least 99% identical to SEQ ID NO: 34

In another embodiment, the anti-LAG-3 antibody has a light chain variable domain (VL) that is at least 90% identical in sequence to the VL amino acid sequence of SEQ ID NO: 28, e.g. at least 92% identical, such as at least 95%, at least 96%, at least 97%, at least 98% or at least 99% identical to SEQ ID NO: 28; and a light chain constant region amino acid sequence that is at least 90% identical in sequence to SEQ ID NO: 32, e.g. at least 92% identical, such as at least 95%, at least 96%, at least 97%, at least 98% or at least 99% identical to SEQ ID NO: 32.

In another embodiment, the anti-LAG-3 antibody has a light chain that comprises any one of SEQ ID NOs: 4, 8, 12, 16, 20 or 24, and SEQ ID NO: 34.

In another embodiment, the anti-LAG-3 antibody has a light chain that comprises SEQ ID NO: 28 and SEQ ID NO: 32.

In certain embodiments, the anti-LAG-3 antibody comprises any one of the above-described heavy chains and any one of the above-described light chains.

In some embodiments, the anti-LAG-3 antibody or antigen-binding portion of the invention comprises the H-CDR1-3 and L-CDR1-3 amino acid sequences of:

a) SEQ ID NOs: 35, 36, 37, 38, 39, and 40, respectively;
b) SEQ ID NOs: 41, 42, 43, 44, 45, and 40, respectively;
c) SEQ ID NOs: 35, 42, 46, 44, 47, and 40, respectively;
d) SEQ ID NOs: 48, 49, 50, 51, 47, and 52, respectively;
e) SEQ ID NOs: 53, 54, 55, 44, 45, and 40, respectively;
f) SEQ ID NOs: 56, 57, 58, 59, 60, and 61, respectively; or
g) SEQ ID NOs: 62, 63, 64, 65, 66, and 67, respectively,

In some embodiments, the anti-LAG-3 antibody or antigen-binding portion of the invention comprises an H-CDR3 and an L-CDR3 that are 80%, 85%, 90%, 91%, 92%, 93%, 94%, 95%, 96%, 97%, 98%, or 99% identical to the amino acid sequences of:

a) SEQ ID NOs: 37 and 40, respectively;
b) SEQ ID NOs: 43 and 40, respectively;
c) SEQ ID NOs: 46 and 40, respectively;
d) SEQ ID NOs: 50 and 52, respectively:
e) SEQ ID NOs. 55 and 40, respectively;
f) SEQ ID NOS: 58 and 61, respectively; or
g) SEQ ID NOS: 64 and 67, respectively.

In some embodiments, the anti-LAG-3 antibody or antigen-binding portion of the invention comprises a VH and a VL that are 80%, 85%, 90%, 91%, 92%, 93%, 94%, 95%, 96%, 97%, 98%, or 99% identical to the amino acid sequences of:

a) SEQ ID NOS: 3 and 4, respectively;
b) SEQ ID NOs: 7 and 8, respectively;
c) SEQ ID NOs: 11 and 12, respectively;
d) SEQ ID NOs: 15 and 16, respectively;
e) SEQ ID NOs: 19 and 20, respectively;
f) SEQ ID NOs: 23 and 24, respectively; or
g) SEQ ID NOs: 27 and 28, respectively.

In some embodiments, the anti-LAG-3 antibody or antigen-binding portion of the invention comprises a VH and a VL that have the amino acid sequences of:

a) SEQ ID NOs: 3 and 4, respectively;
b) SEQ ID NOs: 7 and 8, respectively:
c) SEQ ID NOs: 11 and 12, respectively;
d) SEQ ID NOs: 15 and 16, respectively;
e) SEQ ID NOs: 19 and 20, respectively;
f) SEQ ID NOS: 23 and 24, respectively; or
g) SEQ ID NOs: 27 and 28, respectively.

In some embodiments, the anti-LAG-3 antibody comprises:

a) an HC with the amino acid sequences of SEQ ID NOs: 3 and 30 and an LC with the amino acid sequences of SEQ ID NOs: 4 and 34;
b) an HC with the amino acid sequences of SEQ ID NOs: 7 and 30 and an LC with the amino acid sequences of SEQ ID NOs: 8 and 34;
c) an HC with the amino acid sequences of SEQ ID NOs: 11 and 30 and an LC with the amino acid sequences of SEQ ID NOs: 12 and 34;
d) an HC with the amino acid sequences of SEQ ID NOs: 15 and 30 and an LC with the amino acid sequences of SEQ ID NOS: 16 and 34;
e) an HC with the amino acid sequences of SEQ ID NOs: 19 and 30 and an LC with the amino acid sequences of SEQ ID NOs: 20 and 34;
f) an HC with the amino acid sequences of SEQ ID NOs: 23 and 30 and an LC with the amino acid sequences of SEQ ID NOs: 24 and 34; or
g) an HC with the amino acid sequences of SEQ ID NOs: 27 and 30 and an LC with the amino acid sequences of SEQ ID NOs: 28 and 32.

In some embodiments, the anti-LAG-3 antibody or antigen-binding portion of the invention comprises the H-CDR1-3 and L-CDR1-3 amino acid sequences of antibody 15646, 15532, 15723, 15595, 15431, 15572 or 15011.

In some embodiments, the anti-LAG-3 antibody or antigen-binding portion of the invention comprises a VH and VL that are at least 90% identical in amino acid sequence to the VH and VL, respectively, of antibody 15646, 15532, 15723, 15595, 15431, 15572 or 15011.

In some embodiments, the anti-LAG-3 antibody or antigen-binding portion of the invention comprises a VH and VL that are the VH and VL, respectively, of antibody 15646, 15532, 15723, 15595, 15431, 15572 or 15011.

In some embodiments, the anti-LAG-3 antibody of the invention is antibody 15646, 15532, 15723, 15595, 15431, 15572 or 15011, or an antibody with the same amino acid sequences as said antibody.

The invention also provides an anti-LAG-3 antibody or an antigen-binding portion thereof that binds to an epitope of human LAG-3 having:

a) 1, 2, 3, 4, 5, 6, or all 7 amino acid residues selected from H85, P86, A87, P89, S91, W92, and G93 of SEQ ID NO: 68 (e.g., antibody 15532);
b) 1, 2, 3, 4 , 5, 6, 7, 8, 9, 10, 11, 12, 13, 14, 15, 16, 17, 18, 19, 20, 21, 22, 23, 24, 25, 26, 27, 28, 29, 30, 31, or all 32 amino acid residues selected from A40, Q41, P43, P46, P49, D52, T62, Q64, H65, Q66, P67, D68, G93, P94, P96, R98, Y99, T100, V101, P106, G107, R119, E124, R129, G130, D131, S133, R137, P138, D143, R148, and R163 of SEQ ID NO: 68 (e.g., antibody 15431);
c) 1, 2, 3, 4, 5, 6, 7, 8, 9, 10, 11, 12, 13, 14, 15, 16, 17, 18, 19, 20, 21, 22, 23, 24, 25, 26, 27, 28, or all 29 amino acid residues selected from A40, Q41, P43, P46, P49, D52, T62, Q64, H65, Q66, P67, D68, P96, Y99, T100, V101, P106, G107, R119, E124, R129, G130, D131, S133, R137, P138, D143, R148, and R163 of SEQ ID NO: 68 (e.g., antibody 15572); or
d) 1, 2, 3, or all 4 amino acid residues selected from G107, L109, R110, and S111 of SEQ ID NO: 68 (e.g., antibody 15011).

The invention also provides a monoclonal antibody or an antigen-binding portion thereof that binds to an epitope of human LAG-3 having residues 98-105. In some embodiments, the antibody or antigen-binding portion binds to an epitope of human LAG-3 having:

a) two, or three amino acid segments selected from residues 78-105 and 123-131 of SEQ ID NO: 68 (e.g., antibody 15532);
b) one, two, three, four, or five amino acid segments selected from residues 23-30, 40-66, 88-105, 123-137, and 148-152 of SEQ ID NO: 68 (e.g., antibody 15431);
c) one, two, three, four, or five amino acid segments selected from residues 23-30, 40-66, 98-105, 118-137, and 148-161 of SEQ ID NO: 68 (e.g., antibody 15572); or
d) amino acid residues 98-105 of SEQ ID NO: 68 (e.g., antibody 15011).

The invention also provides a monoclonal antibody or an antigen-binding portion thereof that binds to an epitope of human LAG-3 having residues 23-30 and 40-66 of SEQ ID NO: 68. In some embodiments, the epitope further has residues 88-105, 123-137, and/or 148-152 of SEQ ID NO: 68. In some embodiments, the epitope further has residues 98-105, 118-137, and 148-161 of SEQ ID NO: 68.

The invention also provides an anti-LAG-3 antibody or an antigen-binding portion thereof that competes or cross-competes for binding with, or binds to the same epitope as, an antibody selected from the group consisting of 15532, 15646, 15723, 15595, 15431, 15572, and 15011.

In some embodiments, the anti-LAG-3 antibody or antigen-binding portion thereof of the invention does not bind to the same epitope of human LAG-3 as antibody 25F7-Lag3.5.

In some embodiments, the anti-LAG-3 antibody or antigen-binding portion thereof of the invention utilizes a human heavy chain germline gene selected from the group consisting of IGHV4-34, IGHV1-24, IGHV6-1, IGHV4-39, and IGHV3-23. In certain embodiments, the heavy chain germline gene is at least 75%, 80%, 82%, 85%, 86%, 87%, 88%, 89%, 90%, 91%, 92%, 93%, 94%, 95%, 96%, 97%, 98%, 99%, or 100% identical to the corresponding heavy chain sequence in the anti-LAG3 antibody or antigen-binding portion. In certain embodiments, the framework region sequences of said heavy chain germline gene are at least 75%, 80%, 82%, 85%, 86%, 87%, 88%, 89%, 90%, 91%, 92%, 93%, 94%, 95%, 96%, 97%, 98%, 99%, or 100% identical to the corresponding heavy chain framework region sequences in the anti-LAG3 antibody or antigen-binding portion.

In some embodiments, the anti-LAG-3 antibody or antigen-binding portion thereof of the invention utilizes a human light chain germline gene selected from the group consisting of IGKV3-11, IGKV1-12, IGKV1-5, and IGLV3-19. In certain embodiments, the light chain germline gene is at least 75%, 80%, 82%, 85%, 86%, 87%, 88%, 89%, 90%, 91%, 92%, 93%, 94%, 95%, 96%, 97%, 98%, 99%, or 100% identical to the corresponding light chain sequence in the anti-LAG3 antibody or antigen-binding portion. In certain embodiments, the framework region sequences of said light chain germline gene are at least 75%, 80%, 82%, 85%, 86%, 87%, 88%, 89%, 90%, 91%, 92%. 93%, 94%, 95%, 96%, 97%, 98%, 99%, or 100% identical to the corresponding light chain framework region sequences in the anti-LAG3 antibody or antigen-binding portion.

In particular embodiments, the anti-LAG-3 antibody or antigen-binding portion thereof of the invention utilizes any combination of the above human heavy chain germline genes and human light chain germline genes (e.g., IGHV4-34 and IGKV3-11, IGHV1-24 and IGKV1-12, IGHV6-1 and IGKV3-11, IGHV4-39 and IGKV1-5, or IGHV3-23 and IGLV3-19). In some embodiments, the heavy and light chain germline genes are at least 75%, 80%, 82%, 85%, 86%, 87%, 88%, 89%, 90%, 91%, 92%, 93%, 94%, 95%, 96%, 97%, 98%, 99%, or 100% identical to the corresponding heavy and light chain sequences, respectively, in the anti-LAG3 antibody or antigen-binding portion. In certain embodiments, the framework region sequences of said heavy and light chain germline genes are at least 75%, 80%, 82%, 85%, 86%, 87%, 88%, 89%, 90%, 91%, 92%, 93%, 94%, 95%, 96%, 97%, 98%, 99%, or 100% identical to the corresponding heavy and light chain framework region sequences, respectively, in the anti-LAG3 antibody or antigen-binding portion.

In some embodiments, any of the anti-LAG-3 antibodies or antigen-binding portions described herein may bind to human LAG-3 with an EC50 of, for example, 0.2 nM or less. 0.15 nM or less, 0.1 nM or less, 0.09 nM or less. 0.08 nM or less, 0.07 nM or less. 0.06 nM or less. 0.05 nM or less, or 0.04 nM or less. In some embodiments. any of the anti-LAG-3 antibodies or antigen-binding portions described herein may bind to cynomolgus LAG-3 with, for example, an EC50 of 0.4 nM or less, 0.3 nM or less, 0.2 nM or less, 0.1 nM or less, 0.09 nM or less, 0.08 nM or less. 0.07 nM or less, 0.06 nM or less, 0.05 nM or less. 0.04 nM or less, or 0.03 nM or less.

In some embodiments, any of the anti-LAG-3 antibodies or antigen-binding portions described herein may bind to human LAG-3 with an EC50 of, for example, 0.1 nM or less. In some embodiments, any of the anti-LAG-3 antibodies or antigen-binding portions described herein may bind to cynomolgus LAG-3 with, for example, an EC50 of 0.3 nM or less. In particular embodiments, any of the anti-LAG-3 antibodies or antigen-binding portions described herein may bind to human LAG-3 with, for example, an EC50 of 0.1 nM or less and cynomolgus LAG-3 with, for example, an EC50 of 0.3 nM or less.

In some embodiments, any of the anti-LAG-3 antibodies or antigen-binding portions described herein may inhibit binding of ligands such as MHC class II (MHCII) or LSECtin to LAG-3. For example, at 20 μg/mL, the anti-LAG-3 antibody or antigen-binding portion may reduce the binding of LAG-3 to MHCII by at least 35%, at least 40%, at least 45%, at least 50%, at least 55%, at least 60%, at least 65%, at least 70%, at least 75%, at least 80%, at least 85%, at least 89%, at least 90%, at least 91%, at least 92%, at least 93%, at least 94%, at least 95%, at least 96%, at least 97%, at least 98%, at least 99%, or 100% compared to binding in the presence of a negative control antibody. In one embodiment, the anti-LAG-3 antibody or antigen-binding protein may reduce the binding of LAG-3 to MHCII by great than 85% compared to the negative control. In one embodiment, the anti-LAG-3 antibody or antigen-binding protein may reduce the binding of LAG-3 to MHCII by between about 35% and 85% compared to the negative control.

In some embodiments, any of the anti-LAG-3 antibodies or antigen-binding portions described herein may block binding between LAG-3 and MHC class II, e.g., human LAG-3 expressed on Jurkat cells and human MHC class II expressed on Raji cells (for example, at a concentration of 0.1 μg/mL, 0.5 μg/mL, 1 μg/mL, 5 μg/mL, 10 μg/mL, 20 μg/mL, 30 μg/mL, 40 μg/mL, or 50 μg/mL).

In some embodiments, any of the anti-LAG-3 antibodies or antigen-binding portions described herein may bind to human LAG-3 with a K_{D} of 5.0×10⁻⁸ or less, 4.0×10⁻⁸ or less, 3.0×10⁻⁸ or less, 2.0×10⁻⁸ or less, 1.0×10⁻⁸ or less, 9.0×10⁻⁹ or less, 8.0×10⁻⁹ or less, 7.0×10⁻⁹ or less, 6.0×10⁻⁹ or less. 5.0×10⁻⁹ or less, 4.0×10⁻⁹ or less, 3.0×10⁻⁹ or less, 2.0×10⁻⁹ or less, or 1.0×10⁻⁹ or less, as measured by surface plasmon resonance.

In some embodiments, any of the anti-LAG-3 antibodies or antigen-binding portions described herein may bind to cynomolgus LAG-3 with a K_{D} of 1.5×10⁻⁷ or less, 1.0×10⁻⁷ or less, 9.0×10⁻⁸ or less, 8.0×10⁻⁸ or less, 7 0×10⁻⁸ or less, 6.0×10⁻⁸ or less, 5.0×10⁻⁸ or less, 4.0×10⁻⁸ or less, 3.0×10⁻⁸ or less, 2.0×10⁻⁸ or less, or 1.0×10⁻⁸ or less, as measured by surface plasmon resonance.

In some embodiments, any of the anti-LAG-3 antibodies or antigen-binding portions described herein may bind to mouse LAG-3 with a K_{D} of 5.0×10⁻or less, 4.5×10⁻⁸ or less, 4.0×10⁻⁸ or less. 3.5×10⁻⁸ or less, or 3.0×10⁻⁸ or less. as measured by surface plasmon resonance.

In some embodiments, any of the anti-LAG-3 antibodies or antigen-binding portions described herein may stimulate IL-2 production, e.g., from SEB-stimulated PBMCs.

In some embodiments, any of the anti-LAG-3 antibodies or antigen-binding portions described herein may reduce cellular and/or soluble levels of LAG-3, e.g., in a human T cell line (such as a human T cell line overexpressing LAG-3).

In some embodiments, any of the anti-LAG-3 antibodies or antigen-binding portions described herein may induce tumor growth regression and/or delay tumor growth in vivo.

In some embodiments, any of the anti-LAG-3 antibodies or antigen-binding portions described herein may bind to a different epitope of human LAG-3 than antibody 25F7-Lag3.5.

In one embodiment, administration of an anti-LAG-3 antibody of the invention or an antigen-binding portion thereof may activate T-cells, causing enhanced anti-tumor activity.

The class of an anti-LAG-3 antibody obtained by the methods described herein may be changed or switched with another class or subclass. In one aspect of the invention, a nucleic acid molecule encoding VL or VH is isolated using methods well-known in the art such that it does not include nucleic acid sequences encoding CL or CH, respectively. The nucleic acid molecules encoding VL or VH then are operatively linked to a nucleic acid sequence encoding a CL or CH, respectively, from a different class of immunoglobulin molecule. This may be achieved using a vector or nucleic acid molecule that comprises a CL or CH chain, as described above. For example, an anti-LAG-3 antibody that was originally IgM may be class switched to IgG. Further, the class switching may be used to convert one IgG subclass to another, e.g., from IgG₁ to IgG₂. A κ light chain constant region can be changed, e.g., to a λ light chain constant region. A preferred method for producing an antibody of the invention with a desired Ig isotype comprises the steps of isolating a nucleic acid molecule encoding the heavy chain of an anti-LAG-3 antibody and a nucleic acid molecule encoding the light chain of an anti-LAG-3 antibody, obtaining the variable domain of the heavy chain, ligating the variable domain of the heavy chain with the constant region of a heavy chain of the desired isotype. expressing the light chain and the ligated heavy chain in a cell, and collecting the anti-LAG-3 antibody with the desired isotype.

The anti-LAG-3 antibody of the invention can be an IgG, an IgM, an IgE, an IgA, or an IgD molecule. but is typically of the IgG isotype, e.g. of IgG subclass IgG₁, IgG₂a or IgG₂b, IgG₃ or IgG₄. In one embodiment, the antibody is an IgG₁. In another embodiment, the antibody is an IgG₂. In certain embodiments, the IgG₁ antibodies of the present invention that bind to a LAG-3 epitope described herein offer superior activity in modulating (e.g., inhibiting) LAG-3 functions to achieve the cancer treatment or immunostimulatory effects.

In one embodiment, the anti-LAG-3 antibody may comprise at least one mutation in the Fc region. A number of different Fc mutations are known, where these mutations provide altered effector function. For example, in many cases it will be desirable to reduce or eliminate effector function, e.g. where ligand/receptor interactions are undesired or in the case of antibody-drug conjugates.

In one embodiment, the anti-LAG-3 antibody comprises at least one mutation in the Fc region that reduces effector function. Fc region amino acid positions that may be advantageous to mutate in order to reduce effector function include one or more of positions 228, 233, 234 and 235, where amino acid positions are numbered according to the IMGT® numbering scheme.

In one embodiment, one or both of the amino acid residues at positions 234 and 235 may be mutated, for example from Leu to Ala (L234A/L235A). These mutations reduce effector function of the Fc region of IgG₁ antibodies. Additionally or alternatively, the amino acid residue at position 228 may be mutated, for example to Pro. In some embodiments, the amino acid residue at position 233 may be mutated, e.g., to Pro, the amino acid residue at position 234 may be mutated, e.g., to Val, and/or the amino acid residue at position 235 may be mutated, e.g., to Ala. The amino acid positions are numbered according to the IMGT® numbering scheme.

In some embodiments, where the antibody is of the IgG₄ subclass, it may comprise the mutation S228P, i.e. having a proline in position 228, where the amino acid position is numbered according to the IMGT® numbering scheme. This mutation is known to reduce undesired Fab arm exchange.

In certain embodiments, an antibody or antigen-binding portion thereof of the invention may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov et al., Human Antibodies and Hybridomas 6: 93-101 (1995)) and use of a cysteine residue, a marker peptide and a C-terminal polyhistidine tag to make bivalent and biotinylated scFv molecules (Kipriyanov et al., Mol. Immunol. 31: 1047-1058 (1994). Other examples include where one or more CDRs from an antibody are incorporated into a molecule either covalently or noncovalently to make it an immunoadhesin that specifically binds to an antigen of interest. In such embodiments, the CDR(s) may be incorporated as part of a larger polypeptide chain, may be covalently linked to another polypeptide chain, or may be incorporated noncovalently.

In another embodiment, a fusion antibody or immunoadhesin may be made that comprises all or a portion of an anti-LAG-3 antibody of the invention linked to another polypeptide. In certain embodiments, only the variable domains of the anti-LAG-3 antibody are linked to the polypeptide. In certain embodiments, the VH domain of an anti-LAG-3 antibody is linked to a first polypeptide, while the VL domain of an anti-LAG-3 antibody is linked to a second polypeptide that associates with the first polypeptide in a manner such that the VH and VL domains can interact with one another to form an antigen-binding site. In another preferred embodiment, the VH domain is separated from the VL domain by a linker such that the VH and VL domains can interact with one another (e.g., single-chain antibodies). The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bi-specific antibody

To create a single chain antibody (scFv), the VH- and VL-encoding DNA fragments are operatively linked to another fragment encoding a flexible linker. e.g., encoding the amino acid sequence (Gly4 -Ser)3 (SEQ ID NO: 74), such that the VH and VL sequences can be expressed as a contiguous single-chain protein, with the VL and VH domains joined by the flexible linker. See, e.g., Bird et al., Science 242: 423-426 (1988); Huston et al., Proc. Natl. Acad. Sci. USA 85: 5879-5883 (1988); and McCafferty et al., Nature 348: 552-554 (1990). The single chain antibody may be monovalent, if only a single VH and VL are used; bivalent, if two VH and VL are used; or polyvalent, if more than two VH and VL are used. Bi-specific or polyvalent antibodies may be generated that bind specifically to human LAG-3 and to another molecule, for instance.

In other embodiments, other modified antibodies may be prepared using anti-LAG-3 antibody-encoding nucleic acid molecules. For instance, “kappa bodies” (III et al., Protein Eng. 10: 949-57 (1997)), “minibodies” (Martin et al., EMBO J. 13: 5303-9 (1994). “diabodies” (Holliger et al., Proc. Natl. Acad. Sci. USA 90: 6444-6448 (1993)), or “Janusins” (Traunecker et al., EMBO J. 10: 3655-3659 (1991) and Traunecker et al., Int. J. Cancer (Suppl.) 7: 51-52 (1992)) may be prepared using standard molecular biological techniques following the teachings of the specification.

An anti-LAG-3 antibody or antigen-binding portion of the invention can be derivatized or linked to another molecule (e.g., another peptide or protein). In general, the antibodies or portions thereof are derivatized such that LAG-3 binding is not affected adversely by the derivatization or labeling. Accordingly, the antibodies and antibody portions of the invention are intended to include both intact and modified forms of the human anti-LAG-3 antibodies described herein. For example, an antibody or antibody portion of the invention can be functionally linked (by chemical coupling, genetic fusion, noncovalent association or otherwise) to one or more other molecular entities, such as another antibody (e.g., a bi-specific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bi-specific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available, e.g., from Pierce Chemical Company, Rockford, Il.

An anti-LAG-3 antibody can also be derivatized with a chemical group such as polyethylene glycol (PEG), a methyl or ethyl group, or a carbohydrate group. These groups may be useful to improve the biological characteristics of the antibody, e.g., to increase serum half-life.

An antibody according to the present invention may also be labeled. As used herein, the terms “label” or “labeled” refer to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine. mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

In certain embodiments, the antibodies of the invention may be present in a neutral form (including zwitter ionic forms) or as a positively or negatively-charged species. In some embodiments, the antibodies may be complexed with a counterion to form a pharmaceutically acceptable salt.

The term “pharmaceutically acceptable salt” refers to a complex comprising one or more antibodies and one or more counterions, wherein the counterions are derived from pharmaceutically acceptable inorganic and organic acids and bases.

### Bi-Specific Binding Molecules

In a further aspect, the invention provides a bi-specific binding molecule having the binding specificity (e.g., comprising the antigen-binding portions) of an anti-LAG-3 antibody described herein and the binding specificity of another anti-LAG-3 antibody (e.g., another anti-LAG-3 antibody described herein) or an antibody that targets a different protein, such as another immune checkpoint protein, a cancer antigen, or another cell surface molecule whose activity mediates a disease condition such as cancer. Such bi-specific binding molecules are known in the art, and examples of different types of bi-specific binding molecules are given elsewhere herein.

### Nucleic Acid Molecules and Vectors

The present invention also provides nucleic acid molecules and sequences encoding anti-LAG-3 antibodies or antigen-binding portions thereof described herein. In some embodiments, different nucleic acid molecules encode the heavy chain and light chain amino acid sequences of the anti-LAG-3 antibody or an antigen-binding portion thereof. In other embodiments, the same nucleic acid molecule encodes the heavy chain and light chain amino acid sequences of the anti-LAG-3 antibody or an antigen-binding portion thereof.

A reference to a nucleotide sequence encompasses its complement unless otherwise specified. Thus, a reference to a nucleic acid having a particular sequence should be understood to encompass its complementary strand, with its complementary sequence. The term “polynucleotide” as referred to herein means a polymeric form of nucleotides of at least 10 bases in length, either ribonucleotides or deoxynucleotides or a modified form of either type of nucleotide. The term includes single- and double- stranded forms.

The invention also provides nucleotide sequences that are at least 70%, 75%, 80%, 85%, 90%, 95%, 97%, 98% or 99% identical to one or more nucleotide sequences recited herein, e.g., to a nucleotide sequence selected from the group consisting of SEQ ID NOs: 1, 2, 5, 6, 9, 10, 13, 14, 17, 18, 21, 22, 25 and 26, or to a nucleotide sequence encoding an amino acid sequence selected from the group consisting of SEQ ID NOs: 3, 4, 7, 8, 11, 12, 15, 16, 19, 20, 23, 24, 27 and 28. The term “percent sequence identity” in the context of nucleic acid sequences refers to the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wisconsin. FASTA, which includes, e.g., the programs FASTA2 and FASTA3, provides alignments and percent sequence identity of the regions of the best overlap between the query and search sequences (see, e.g., Pearson, Methods Enzymol. 183: 63-98 (1990); Pearson, Methods Mol. Biol. 132: 185-219 (2000); Pearson, Methods Enzymol. 266: 227-258 (1996); and Pearson, J. Mol. Biol. 276: 71-84 (1998); incorporated herein by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, incorporated herein by reference.

In one aspect, the invention provides a nucleic acid molecule comprising a nucleotide sequence selected from the group consisting of SEQ ID NOs: 1, 2, 5, 6, 9, 10, 13, 14, 17, 18, 21, 22, 25 and 26. In certain embodiments, the nucleic acid molecule comprises the nucleotide sequences of SEQ ID NOs: 1 and 2, SEQ ID NOs: 5 and 6, SEQ ID NOs: 9 and 10, SEQ ID NOs: 13 and 14, SEQ ID NOs: 17 and 18, SEQ ID NOs: 21 and 22, or SEQ ID NOs: 25 and 26.

In any of the above embodiments, the nucleic acid molecules may be isolated.

In a further aspect, the present invention provides a vector suitable for expressing one of the chains of an antibody or antigen-binding portion thereof as described herein. The term “vector”, as used herein, means a nucleic acid molecule capable of transporting another nucleic acid to which it has been linked. In some embodiments, the vector is a plasmid, i.e., a circular double stranded piece of DNA into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The invention provides vectors comprising nucleic acid molecules that encode the heavy chain of an anti-LAG-3 antibody of the invention or an antigen-binding portion thereof, the light chain of an anti-LAG-3 antibody of the invention or an antigen-binding portion thereof, or both the heavy and light chains of an anti-LAG-3 antibody of the invention or an antigen-binding portion thereof. The invention further provides vectors comprising nucleic acid molecules encoding fusion proteins, modified antibodies, antibody fragments, and probes thereof.

A nucleic acid molecule encoding the heavy and/or light chain of an anti-LAG-3 antibody or antigen-binding portion thereof of the invention can be isolated from any source that produces such an antibody or portion. In various embodiments, the nucleic acid molecules are isolated from B cells that express an anti-LAG-3 antibody isolated from an animal immunized with a human LAG-3 antigen, or from an immortalized cell produced from such a B cell. Methods of isolating nucleic acids encoding an antibody are well-known in the art. mRNA may be isolated and used to produce cONA for use in polymerase chain reaction (PCR) or cDNA cloning of antibody genes. In certain embodiments, a nucleic acid molecule of the invention can be synthesized rather than isolated.

In some embodiments, a nucleic acid molecule of the invention can comprise a nucleotide sequence encoding a VH domain from an anti-LAG-3 antibody or antigen-binding portion of the invention joined in-frame to a nucleotide sequence encoding a heavy chain constant region from any source. Similarly, a nucleic acid molecule of the invention can comprise a nucleotide sequence encoding a VL domain from an anti-LAG-3 antibody or antigen-binding portion of the invention joined in-frame to a nucleotide sequence encoding a light chain constant region from any source.

In a further aspect of the invention, nucleic acid molecules encoding the variable domain of the heavy (VH) and/or light (VL) chains may be “converted” to full-length antibody genes. In one embodiment, nucleic acid molecules encoding the VH or VL domains are converted to full-length antibody genes by insertion into an expression vector already encoding heavy chain constant (CH) or light chain constant (CL) regions. respectively, such that the VH segment is operatively linked to the CH segment(s) within the vector, and/or the VL segment is operatively linked to the CL segment within the vector. In another embodiment, nucleic acid molecules encoding the VH and/or VL domains are converted into full-length antibody genes by linking, e.g., ligating, a nucleic acid molecule encoding a VH and/or VL domains to a nucleic acid molecule encoding a CH and/or CL region using standard molecular biological techniques. Nucleic acid molecules encoding the full-length heavy and/or light chains may then be expressed from a cell into which they have been introduced and the anti-LAG-3 antibody isolated.

The nucleic acid molecules may be used to recombinantly express large quantities of anti-LAG-3 antibodies. The nucleic acid molecules also may be used to produce chimeric antibodies, bi-specific antibodies, single chain antibodies, immunoadhesins, diabodies, mutated antibodies and antibody derivatives, as described herein.

In another embodiment, a nucleic acid molecule of the invention is used as a probe or PCR primer for a specific antibody sequence. For instance, the nucleic acid can be used as a probe in diagnostic methods or as a PCR primer to amplify regions of DNA that could be used, inter alia, to isolate additional nucleic acid molecules encoding variable domains of anti-LAG-3 antibodies. In some embodiments, the nucleic acid molecules are oligonucleotides. In some embodiments, the oligonucleotides are from highly variable domains of the heavy and light chains of the antibody of interest. In some embodiments. the oligonucleotides encode all or a part of one or more of the CDRs of the anti-LAG-3 antibodies or antigen-binding portions thereof of the invention as described herein.

In another embodiment, the nucleic acid molecules and vectors may be used to make mutated anti-LAG-3 antibodies. The antibodies may be mutated in the variable domains of the heavy and/or light chains, e.g., to alter a binding property of the antibody. For example, a mutation may be made in one or more of the CDRs o increase or decrease the K_{D} of the anti-LAG-3 antibody, to increase or decrease Koli, or to alter the binding specificity of the antibody. In another embodiment, one or more mutations are made at an amino acid residue that is known to be changed compared to the germline in a monoclonal antibody of the invention. The mutations may be made in a CDR or framework region of a variable domain, or in a constant region. In a preferred embodiment, the mutations are made in a variable domain. In some embodiments, one or more mutations are made at an amino acid residue that is known to be changed compared to the germline in a CDR or framework region of a variable domain of an antibody or antigen-binding portion thereof of the invention.

In another embodiment, the framework region(s) are mutated so that the resulting framework region(s) have the amino acid sequence of the corresponding germline gene. A mutation may be made in a framework region or constant region to increase the half-life of the anti-LAG-3 antibody. See, e.g., PCT Publication WO 00/09560. A mutation in a framework region or constant region also can be made to alter the immunogenicity of the antibody, and/or to provide a site for covalent or non-covalent binding to another molecule. According to the invention, a single antibody may have mutations in any one or more of the CDRs or framework regions of the variable domain or in the constant region.

In some embodiments, the anti-LAG-3 antibodies of the invention or antigen-binding portions thereof are expressed by inserting DNAs encoding partial or full-length light and heavy chains, obtained as described above, into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs. EBV derived episomes, and the like. The antibody coding sequence may be ligated into a vector such that transcriptional and translational control sequences within the vector serve their intended function of regulating the transcription and translation of the antibody coding sequence. The expression vector and expression control sequences may be chosen to be compatible with the expression host cell used. The antibody light chain coding sequence and the antibody heavy chain coding sequence can be inserted into separate vectors, and may be operatively linked to the same or different expression control sequences (e.g., promoters). In one embodiment, both coding sequences are inserted into the same expression vector and may be operatively linked to the same expression control sequences (e.g., a common promoter), to separate identical expression control sequences (e.g., promoters), or to different expression control sequences (e.g., promoters). The antibody coding sequences may be inserted into the expression vector by standard methods (e.g., ligation of complementary restriction sites on the antibody gene fragment and vector, or blunt end ligation if no restriction sites are present).

A convenient vector is one that encodes a functionally complete human CH or CL immunoglobulin sequence, with appropriate restriction sites engineered so that any VH or VL sequence can easily be inserted and expressed, as described above. The HC- and LC-encoding genes in such vectors may contain intron sequences that will result in enhanced overall antibody protein yields by stabilizing the related mRNA. The intron sequences are flanked by splice donor and splice acceptor sites, which determine where RNA splicing will occur. Location of intron sequences can be either in variable or constant regions of the antibody chains, or in both variable and constant regions when multiple introns are used. Polyadenylation and transcription termination may occur at native chromosomal sites downstream of the coding regions. The recombinant expression vector also can encode a signal peptide that facilitates secretion of the antibody chain from a host cell. The antibody chain gene may be cloned into the vector such that the signal peptide is linked in-frame to the amino terminus of the immunoglobulin chain. The signal peptide can be an immunoglobulin signal peptide or a heterologous signal peptide (i.e., a signal peptide from a non-immunoglobulin protein).

In addition to the antibody chain genes, the recombinant expression vectors of the invention may carry regulatory sequences that control the expression of the antibody chain genes in a host cell. It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells. such as promoters and/or enhancers derived from retroviral LTRs. cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615. Methods for expressing antibodies in plants, including a description of promoters and vectors, as well as transformation of plants, are known in the art. See, e.g., U.S. Pat. No. 6,517,529. Methods of expressing polypeptides in bacterial cells or fungal cells, e.g., yeast cells, are also well known in the art.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. For example, selectable marker genes include the dihydrofolate reductase (DHFR) gene (for use in dhfr-host cells with methotrexate selection/amplification), the neo gene (for G418 selection), and the glutamate synthetase gene.

The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize Cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes. generally. such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

### Host Cells and Methods of Antibody and Antibody Composition Production

An additional aspect of the invention relates to methods for producing the antibody compositions and antibodies and antigen-binding portions thereof of the invention. One embodiment of this aspect of the invention relates to a method for producing an antibody as defined herein, comprising providing a recombinant host cell capable of expressing the antibody, cultivating said host cell under conditions suitable for expression of the antibody, and isolating the resulting antibody. Antibodies produced by such expression in such recombinant host cells are referred to herein as “recombinant antibodies.” The invention also provides progeny cells of such host cells, and antibodies produced by same.

The term “recombinant host cell” (or simply “host cell”), as used herein, means a cell into which a recombinant expression vector has been introduced. The invention provides host cells that may comprise, e.g., a vector according to the invention described above. The invention also provides host cells that comprise, e.g., a nucleotide sequence encoding the heavy chain or an antigen-binding portion thereof, a nucleotide sequence encoding the light chain or an antigen-binding portion thereof, or both, of an anti-LAG-3 antibody or antigen-binding portion thereof of the invention. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

Nucleic acid molecules encoding anti-LAG-3 antibodies and vectors comprising these nucleic acid molecules can be used for transfection of a suitable mammalian, plant, bacterial or yeast host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455. Methods of transforming plant cells are well known in the art, including, e.g., Agrobacterium-mediated transformation, biolistic transformation, direct injection, electroporation and viral transformation. Methods of transforming bacterial and yeast cells are also well known in the art.

Mammalian cell lines available as hosts for expression are well known in the art and include many immortalized cell lines available from the American Type Culture Collection (ATCC). These include, inter alia, Chinese hamster ovary (CHO) cells, NSO cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines.

Cell lines of particular preference are selected by determining which cell lines have high expression levels. Other cell lines that may be used are insect cell lines, such as Sf9 or Sf21 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Plant host cells include, e.g., Nicotiana, Arabidopsis, duckweed, com, wheat, potato, etc. Bacterial host cells include E. coli and Streptomyces species. Yeast host cells include Schizosaccharomyces pombe, Saccharomyces cerevisiae and Pichia pastoris.

Further, expression of antibodies of the invention or antigen-binding portions thereof from production cell lines can be enhanced using a number of known techniques. For example, the glutamine synthetase gene expression system (the GS system) is a common approach for enhancing expression under certain conditions. The GS system is discussed in whole or part in connection with EP Patents 0 216 846, 0 256 055, 0 323 997 and 0 338 841.

It is likely that antibodies expressed by different cell lines or in transgenic animals will have different glycosylation patterns from each other. However, all antibodies encoded by the nucleic acid molecules provided herein, or comprising the amino acid sequences provided herein are part of the instant invention, regardless of the glycosylation state of the antibodies, and more generally, regardless of the presence or absence of post-translational modification(s).

### Pharmaceutical Compositions

Another aspect of the invention is a pharmaceutical composition comprising as an active ingredient (or as the sole active ingredient) an anti-LAG-3 antibody or antigen-binding portion thereof, bi-specific binding molecule, or antibody composition of the invention. The pharmaceutical composition may comprise any anti-LAG-3 antibody or antigen-binding portion thereof, bi-specific binding molecule, or antibody composition as described herein. In some embodiments, the pharmaceutical compositions are intended for amelioration, prevention, and/or treatment of a LAG-3-related disorder and/or cancer. As used herein, a LAG-3-related or -mediated disorder refers to a disorder, disease or condition that improves, or slows down in its progression, by modulation of LAG-3 activity. In some embodiments, the compositions are intended for activation of the immune system. In certain embodiments, the compositions are intended for amelioration, prevention, and/or treatment of cancer originating in tissues such as skin, lung, intestine. colon, ovary, brain. prostate, kidney, soft tissues, the hematopoietic system, head and neck, liver, bladder, breast, stomach, uterus and pancreas. In certain embodiments, the cancer is fibrosarcoma, lung carcinoma, or melanoma. In certain embodiments, the cancer is glioblastoma, gliosarcoma, or colorectal cancer. In certain embodiments, the pharmaceutical compositions of the invention are intended for treatment of psoriasis.

Generally, the antibodies, antigen-binding portions, and bi-specific binding molecules of the invention are suitable to be administered as a formulation in association with one or more pharmaceutically acceptable excipient(s), e.g., as described below.

Pharmaceutical compositions of the invention will comprise one or more anti-LAG-3 antibodies, binding portions, or bi-specific binding molecules of the invention, e.g., one or two anti-LAG-3 antibodies, binding portions, or bi-specific binding molecules. In one embodiment, the composition comprises a single anti-LAG-3 antibody of the invention or binding portion thereof.

In another embodiment, the pharmaceutical composition may comprise at least one anti-LAG-3 antibody or antigen-binding portion thereof, e.g., one anti-LAG-3 antibody or portion, and one or more additional antibodies that target one or more relevant cell surface receptors, e.g., one or more cancer-relevant receptors.

The term “excipient” is used herein to describe any ingredient other than the compound(s) of the invention. The choice of excipient(s) will to a large extent depend on factors such as the particular mode of administration, the effect of the excipient on solubility and stability, and the nature of the dosage form. As used herein. “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Pharmaceutical compositions of the present invention and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP (good manufacturing practices) conditions.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Any method for administering peptides, proteins or antibodies accepted in the art may suitably be employed for the antibodies and antigen-binding portions of the invention.

The pharmaceutical compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intraarterial, intrathecal, intraventricular, intraurethral, intracranial, intratumoral, and intrasynovial injection or infusions; and kidney dialytic infusion techniques. Regional perfusion is also contemplated. Particular embodiments include the intravenous and the subcutaneous routes.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions, Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

For example, in one aspect, sterile injectable solutions can be prepared by incorporating the anti-LAG-3 antibody, antigen-binding portion thereof, bi-specific binding molecule, or antibody composition in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example. monostearate salts and gelatin, and/or by using modified-release coatings (e.g., slow-release coatings).

The antibodies of the invention can also be administered intranasally or by inhalation, typically in the form of a dry powder (either alone, as a mixture, or as a mixed component particle, for example, mixed with a suitable pharmaceutically acceptable excipient) from a dry powder inhaler, as an aerosol spray from a pressurised container, pump. spray, atomiser (preferably an atomiser using electrohydrodynamics to produce a fine mist), or nebuliser, with or without the use of a suitable propellant, or as nasal drops.

The pressurised container, pump, spray, atomizer, or nebuliser generally contains a solution or suspension of an antibody of the invention comprising, for example, a suitable agent for dispersing, solubilising, or extending release of the active, a propellant(s) as solvent.

Prior to use in a dry powder or suspension formulation, the drug product is generally micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules, blisters and cartridges for use in an inhaler or insufflator may be formulated to contain a powder mix of the compound of the invention, a suitable powder base and a performance modifier.

A suitable solution formulation for use in an atomiser using electrohydrodynamics to produce a fine mist may contain a suitable dose of the antibody of the invention per actuation and the actuation volume may for example vary from 1 μL to 100 μL.

Formulations for inhaled/intranasal administration may be formulated to be immediate and/or modified release. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” of an antibody of the invention The overall daily dose will typically be administered in a single dose or, more usually, as divided doses throughout the day.

The antibodies and antibody portions of the invention may also be formulated for an oral route administration. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

### Therapeutic Uses of Antibodies and Compositions of the Invention

In one aspect, the anti-LAG-3 antibodies and antigen-binding portions thereof, anti-LAG-3 compositions, and bi-specific binding molecules of the invention are used to enhance or activate the immune system in a human in need thereof. In some embodiments, the patient is immune-suppressed. For example, a physician can boost the anti-cancer activity of a patient's own immune system by administering an anti-LAG-3 antibody of the present invention, alone or in combination with other therapeutic agents (sequentially or concurrently). The LAG-3 antibody modulates the activity of LAG-3 in immune cells, resulting in enhancement of anti-cancer immunity.

In certain embodiments. the antibody or antigen-binding portion thereof, composition, or bi-specific binding molecule is for use in the treatment of cancer, e.g., cancers that originate in tissues such as skin, lung, intestine, colon, ovary, brain, prostate, kidney, soft tissues, the hematopoietic system, head and neck, liver, bladder, breast, stomach, uterus and pancreas and any cancers or other conditions which rely on LAG-3 activity and/or in which the patient expresses or overexpresses a LAG-3 ligand (e.g., MHCII, LSECtin, or both).

In some embodiments, cancers treated by the anti-LAG-3 antibodies, antigen-binding portions, bi-specific binding molecules, and/or antibody compositions of the invention may include, e.g., melanoma (e.g., advanced or metastatic melanoma), non-small cell lung cancer, head and neck squamous cell cancer, renal cell carcinoma, Hodgkin's lymphoma, non-Hodgkin's lymphoma, glioblastoma, glioma, squamous cell lung cancer, small-cell lung cancer, hepatocellular carcinoma, bladder cancer, upper urinary tract cancer, esophageal cancer, gastroesophageal junction cancer, gastric cancer, liver cancer. colon cancer, colorectal carcinoma, multiple myeloma, sarcomas, acute myeloid leukemia, chronic myeloid leukemia, myelodysplastic syndrome, nasopharyngeal cancer, chronic lymphocytic leukemia, acute lymphoblastic leukemia, small lymphocytic lymphoma, ovarian cancer, gastrointestinal cancer, primary peritoneal cancer, fallopian tube cancer, urothelial cancer, HTLV-associated T-cell leukemia/lymphoma, prostate cancer, genitourinary cancer, meningioma, adrenocortical cancer, gliosarcoma, fibrosarcoma, kidney cancer, breast cancer, pancreatic cancer, endometrial cancer, skin basal cell cancer. cancer of the appendix, biliary tract cancer, salivary gland cancer, advanced Merkel cell cancer, diffuse large B cell lymphoma, follicular lymphoma, mesothelioma, or solid tumors. The cancer may be, e.g., at an early, intermediate, late, or metastatic stage.

In some embodiments, cancers treated by the anti-LAG-3 antibodies, antigen-binding portions, compositions, and/or bi-specific binding molecules of the invention may include, e.g., hematologic malignancies, glioblastoma (e g., recurrent glioblastoma), gliosarcoma, non-small cell lung cancer (e.g., advanced non-small cell lung cancer), colorectal cancer, and solid tumors.

In one aspect, the anti-LAG-3 antibodies and antigen-binding portions thereof, antibody compositions, or bi-specific binding molecules of the invention may be used to treat an immune-mediated disorder such as psoriasis, systemic lupus erythematosis, MLS (sclerosis), Crohn's disease, diabetes mellitus, and/or colitis ulcerotis.

In some embodiments, the anti-LAG-3 antibodies and antigen-binding portions thereof, antibody compositions, or bi-specific binding molecules of the invention may be used to treat viral and/or parasitic infections. e.g., where the pathogens inhibit the host immune response. For example, the pathogen may be, e.g., HIV, hepatitis (A, B, or C), human papilloma virus (HPV), lymphocytic choriomeningitis virus (LCMV), adenovirus, flavivirus, echovirus, rhinovirus, coxsackie virus, cornovirus, respiratory syncytial virus, mumps virus, rotavirus, measles virus, rubella virus, parvovirus, vaccinia virus, human T-cell lymphotrophic virus (HTLV), dengue virus, molluscum virus, poliovirus, rabies virus, John Cunningham (JC) virus, arboviral encephalitis virus, simian immunodeficiency virus (SIV), influenza, herpes, Giardia, malaria, Leishmania, Staphylococcus aureus, or Pseudomonas aeruginosa.

In some embodiments, the anti-LAG-3 antibodies and antigen-binding portions thereof, antibody compositions, or bi-specific binding molecules of the invention may be used to treat a patient who is, or is at risk of being, immunocompromised (e.g., due to chemotherapeutic or radiation therapy).

In some embodiments, the anti-LAG-3 antibodies and antigen-binding portions thereof, antibody compositions, or bi-specific binding molecules of the invention may be used for ex vivo activation and expansion of antigen-specific T cells.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

“Therapeutically effective amount” refers to the amount of the therapeutic agent being administered that will relieve to some extent one or more of the symptoms of the disorder being treated. A therapeutically effective amount of an anti-cancer therapeutic may, for example, result in tumor shrinkage, increased survival, elimination of cancer cells, decreased disease progression, reversal of metastasis, or other clinical endpoints desired by healthcare professionals.

The anti-LAG-3 antibodies or antigen-binding portions thereof, antibody compositions, or bi-specific binding molecules of the invention may be administered alone or in combination with one or more other drugs or antibodies (or as any combination thereof). The pharmaceutical compositions, methods and uses of the invention thus also encompass embodiments of combinations (co-administration) with other active agents, as detailed below.

As used herein, the terms “co-administration”, “co-administered” and “in combination with,” referring to the anti-LAG-3 antibodies and antigen-binding portions thereof, antibody compositions, and bi-specific binding molecules of the invention with one or more other therapeutic agents, is intended to mean, and does refer to and include the following:

a) simultaneous administration of such combination of antibody/antigen-binding portion/antibody composition/bi-specific binding molecule of the invention and therapeutic agent(s) to a patient in need of treatment, when such components are formulated together into a single dosage form which releases said components at substantially the same time to said patient.
b) substantially simultaneous administration of such combination of antibody/antigen-binding portion/antibody composition/bi-specific binding molecule of the invention and therapeutic agent(s) to a patient in need of treatment, when such components are formulated apart from each other into separate dosage forms which are taken at substantially the same time by said patient, whereupon said components are released at substantially the same time to said patient,
c) sequential administration of such combination of antibody/antigen-binding portion/antibody composition/bi-specific binding molecule of the invention and therapeutic agent(s) to a patient in need of treatment, when such components are formulated apart from each other into separate dosage forms which are taken at consecutive times by said patient with a significant time interval between each administration, whereupon said components are released at substantially different times to said patient; and
d) sequential administration of such combination of antibody/antigen-binding portion/antibody composition/bi-specific binding molecule of the invention and therapeutic agent(s) to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

The anti-LAG-3 antibodies and antigen-binding portions thereof, antibody compositions, or bi-specific binding molecules of the invention may be administered without additional therapeutic treatments, i.e., as a stand-alone therapy (monotherapy). Alternatively, treatment with the anti-LAG-3 antibodies and antigen-binding portions thereof, antibody compositions, or bi-specific binding molecules of the invention may include at least one additional therapeutic treatment (combination therapy), e.g., another immunostimulatory agent, an anti-cancer agent, an anti-viral agent, or a vaccine (e.g., a tumor vaccine).

In some embodiments, the antibody or antigen-binding portion thereof, antibody composition, or bi-specific binding molecule may be co-administered or formulated with another medication/drug for the treatment of cancer. The additional therapeutic treatment may comprise, e.g., a chemotherapeutic, anti-neoplastic, or anti-angiogenic agent, a different anti-cancer antibody. and/or radiation therapy.

By combining the antibodies or antigen-binding portions thereof, antibody compositions, or bi-specific binding molecules of the invention with agents known to induce terminal differentiation of cancer cells, the effect may be improved further. Such compounds may, for example, be selected from the group consisting of retinoic acid, trans-retinoic acids, cis-retinoic acids, phenylbutyrate, nerve growth factor, dimethyl sulfoxide, active form vitamin D3, peroxisome proliferator-activated receptor gamma, 12-O-tetradecanoylphorbol 13-acetate, hexamethylene-bis-acetamide, transforming growth factor-beta, butyric acid, cyclic AMP, and vesnarinone. In some embodiments, the compound is selected from the group consisting of retinoic acid, phenylbutyrate, all-trans-retinoic acid and active form vitamin D.

Pharmaceutical articles comprising an anti-LAG-3 antibody or antigen-binding portion thereof, antibody composition, or bi-specific binding molecule of the invention and at least one other agent (e.g., a chemotherapeutic, anti-neoplastic, or anti-angiogenic agent) may be used as a combination treatment for simultaneous, separate or successive administration in cancer therapy. The other agent may by any agent suitable for treatment of the particular cancer in question, for example, an agent selected from the group consisting of alkylating agents. e.g., platinum derivatives such as cisplatin, carboplatin and/or oxaliplatin; plant alkoids, e.g., paclitaxel, docetaxel and/or irinotecan; antitumor antibiotics, e.g., doxorubicin (adriamycin), daunorubicin, epirubicin, idarubicin mitoxantrone, dactinomycin, bleomycin, actinomycin, luteomycin, and/or mitomycin; topoisomerase inhibitors such as topotecan; and/or antimetabolites, e.g., fluorouracil and/or other fluoropyrimidines. In some embodiments, the other agent is dacarbazine or gemcitabine.

An anti-LAG-3 antibody or antigen-binding portion thereof, antibody composition, or bi-specific binding molecule of the invention may also be used in combination with other anti-cancer therapies such as vaccines, cytokines, enzyme inhibitors, immunostimulatory compounds, and T cell therapies. In the case of a vaccine, it may, e.g., be a protein, peptide or DNA vaccine containing one or more antigens which are relevant for the cancer being treated, or a vaccine comprising dendritic cells along with an antigen. Suitable cytokines include, for example, IL-2, IFN-gamma and GM-CSF. An example of a type of enzyme inhibitor that has anti-cancer activity is an indoleamine-2,3-dioxygenase (IDO) inhibitor, for example 1-methyl-D-tryptophan (1-D-MT). Adoptive T cell therapy refers to various immunotherapy techniques that involve expanding or engineering patients' own T cells to recognize and attack their tumors.

It is also contemplated that an anti-LAG-3 antibody or antigen-binding portion thereof, antibody composition, or bi-specific binding molecule of the invention may be used in adjunctive therapy in connection with tyrosine kinase inhibitors. These are synthetic, mainly quinazoline-derived, low molecular weight molecules that interact with the intracellular tyrosine kinase domain of receptors and inhibit ligand-induced receptor phosphorylation by competing for the intracellular Mg-ATP binding site.

In some embodiments, the antibody or antigen-binding portion thereof, antibody composition, or bi-specific binding molecule may be used in combination with another medication/drug that mediates immune system activation, including, but not limited to, an agent that modulates the expression or activity of A2AR, BTLA, B7-H3, 87-H4, CTLA-4, CD27, CD28, CD40, CD47, CD55, CD73, CD122, CD137, CD160, CGEN-15049, CHK1, CHK2, CTLA-3, CEACAM (e.g., CEACAM-1 and/or CEACAM-5), GAL9, GITR, HVEM, LY108, LAIR1, ICOS, IDO, KIR, LAIR1, PD-1/PD-L1/PD-L2, OX40, TIGIT, TIM-3, TGFR-beta, VISTA, LILRB2, CMTM6 and/or 2B4. In certain embodiments, the agent is an antibody or an antigen-binding fragment thereof that binds to one of the above molecules.

It is also contemplated that an anti-LAG-3 antibody or antigen-binding portion thereof, antibody composition, or bi-specific binding molecule of the invention may be used in combination with a cytokine (e.g., IL-1, IL-2, IL-12, IL-15 or IL-21), an EGFR inhibitor, a VEGF inhibitor, etc.

In certain aspects, the antibodies and antigen-binding portions, antibody compositions, or bi-specific binding molecules of the invention may be administered in combination with another inhibitor of the LAG-3 pathway, which may target LAG-3 or one or more of its ligands. Examples of such inhibitors include other anti-LAG-3 antibodies. anti-MHCII antibodies, anti-Galectin-3 antibodies, and anti-LSECtin antibodies. In some embodiments, an anti-LAG-3 antibody or antigen-binding portion thereof, bi-specific antibody, or antibody composition of the invention may be administered in combination with BMS-986016, GSK2831781, REGN3767, BAP050 or BAP050-chi, or LAG525.

It is understood that the antibodies and antigen-binding portions thereof, antibody compositions, and bi-specific binding molecules of the invention may be used in a method of treatment as described herein, may be for use in a treatment as described herein. and/or may be for use in the manufacture of a medicament for a treatment as described herein. The invention also provides kits and articles of manufacture comprising the antibodies and antigen-binding portions thereof, antibody compositions, and bi-specific binding molecules described herein.

### Dose and Route of Administration

The antibodies or antigen-binding portions thereof, antibody compositions, or bi-specific binding molecules of the invention will be administered in an effective amount for treatment of the condition in question, i.e., at dosages and for periods of time necessary to achieve a desired result. A therapeutically effective amount may vary according to factors such as the particular condition being treated. the age, sex and weight of the patient, and whether the antibodies are being administered as a stand-alone treatment or in combination with one or more additional anti-cancer treatments.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the invention are generally dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen are adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of this invention may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens are well-known in the relevant art and would be understood to be encompassed by the skilled artisan once provided the teachings disclosed herein.

It is contemplated that a suitable dose of an antibody, antigen-binding portion, antibody composition, or bi-specific binding molecule of the invention will be in the range of 0.1-100 mg/kg, such as about 0.5-50 mg/kg, e.g., about 1-20 mg/kg. The antibody, antigen-binding portion, antibody composition, or bi-specific binding molecule may for example be administered in a dosage of at least 0.25 mg/kg, e.g., at least 0.5 mg/kg, such as at least 1 mg/kg, e.g., at least 1.5 mg/kg, such as at least 2 mg/kg, e.g., at least 3 mg/kg, such as at least 4 mg/kg, e.g., at least 5 mg/kg; and e.g., up to at most 50 mg/kg. such as up to at the most 30 mg/kg. e.g., up to at the most 20 mg/kg, such as up to at the most 15 mg/kg. Administration will normally be repeated at suitable intervals, e.g., once every week, once every two weeks, once every three weeks, or once every four weeks, and for as long as deemed appropriate by the responsible doctor, who may optionally increase or decrease the dosage as necessary.

An effective amount for tumor therapy may be measured by its ability to stabilize disease progression and/or ameliorate symptoms in a patient, and preferably to reverse disease progression, e.g., by reducing tumor size. The ability of an antibody, antigen-binding portion, antibody composition, or bi-specific binding molecule of the invention to inhibit cancer may be evaluated by in vitro assays, e.g., as described in the examples, as well as in suitable animal models that are predictive of the efficacy in human tumors. Suitable dosage regimens will be selected in order to provide an optimum therapeutic response in each particular situation, for example, administered as a single bolus or as a continuous infusion, and with possible adjustment of the dosage as indicated by the exigencies of each case.

### Diagnostic Uses and Compositions

The antibodies of the present invention also are useful in diagnostic processes (e.g., in vitro, ex vivo). For example, the antibodies can be used to detect and/or measure the level of LAG-3 in a sample from a patient (e.g., a tissue sample, or a body fluid sample such as an inflammatory exudate. blood, serum, bowel fluid, saliva, or urine). Suitable detection and measurement methods include immunological methods such as flow cytometry, enzyme-linked immunosorbent assays (ELISA), chemiluminescence assays, radioimmunoassay, and immunohistology. The invention further encompasseskits (e.g., diagnostic kits) comprising the antibodies described herein.

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry. and protein and nucleic acid chemistry and hybridization described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has, ” “having,” “comprises,” or “comprising.” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: Generation and Screening of anti-LAG-3 Antibody Repertoires

Two anti-LAG-3 antibody repertoires were generated by immunization with LAG-3 extracellular domain (ECD) or LAG-3 ECD Fc fusion protein. One antibody library was prepared from single-cell sorted B cells from immunized OmniRat® rats using Symplex™ antibody discovery technology (Osborn et al., J Immunol, 190(4): 1481-90 (2013); Meijer et al , J Mol Biol. 358(3): 764-72 (2006)). The expression constructs from this antibody repertoire encoded fully human immunoglobulins in IgG₁ format carrying two mutations (L234A/L235A) known to reduce effector function of the Fc region of IgG₁ antibodies (Hezareh et al., J Virol. 75(24): 12161-8 (2001). A second antibody repertoire was constructed using single-cell sorted B cells originating from the lymphoid organs of immunized wild-type chickens (Gallus gallus).

Cloned antibodies from both anti-LAG-3 repertoires were individually transfected and expressed in HEK293 cells using 293fectin™ Transfection Reagent (Invitrogen, Cat. No. 12347-019) in 384-well format, and antibody-containing supernatants were collected on day 6 after transfection,

For cell-based antibody screening, CHO-S cells were transfected in 384-well format to express GPI-anchored human LAG-3 using the FreeStyle™ MAX reagent (Invitrogen, Cat. No. 16447-100), and cells transfected with an irrelevant, GPI-anchored control protein were used as a negative control. In order to allow for a multiplexed screening setup, control cells were labeled using carboxyfluorescein succinimidyl ester (CFSE) and mixed with non-labeled LAG-3-transfected cells at a ratio of 1 to 1, and a density of 1×10⁶ cells per mL. In 384-well plates, 40 μL of this cell mix was mixed with 10 μL of antibody-containing supernatant, and cell-bound antibody was revealed by addition of goat anti-human IgG (H+L) AF647 secondary antibody (Molecular Probes, Cat. No. A21445). In parallel, antibodies were screened for binding to cynomolgus LAG-3 in a similar setup. Samples were obtained using high throughput flow cytometry (iQue® Screener, Intellicyt) and data was analyzed using ForeCyt® software by plotting CFSE vs. human IgG binding (AF647). LAG-3-specific primary hits were identified as antibody clones binding to both human and cynomolgus LAG-3-transfected cells (CSFE negative), but not to control cells (CFSE positive), and plate numbers and plate coordinates were collected for hit picking and subsequent sequence analysis.

The heavy and light chain variable region DNA and protein sequences of six functional OmniRat®-derived anti-LAG-3 antibodies (15646, 15532, 15723, 15595, 15431, and 15572) and one functional chicken-derived anti-LAG-3 antibody (15011; see Example 2) are provided in the sequence list section below. An overview of the SEQ ID numbers of the variable and constant region DNA and protein sequences is provided in Tables 8 and 9. SEQ ID numbers for the CDRs are found in Table 9. The CDR sequences herein were determined according to the IMGT® definitions for CDR1 and CDR2. For heavy and light chain CDR3. the definitions herein include one extra amino acid residue upstream of the IMGT-CDR3 (Cys) and one extra amino acid residue downstream (Trp for H-CDR3, Phe for L-CDR3). Germline gene usage of the six OmniRat®-derived antibodies is shown in Table 10.

### Example 2: Humanization of Chicken-Derived Anti-LAG-3 Antibodies

Humanization of the framework regions of the chicken-derived anti-LAG-3 antibodies was performed in order to produce antibody molecules having minimal immunogenicity when administered to humans, while substantially retaining the specificity and affinity of the parental chicken antibodies.

Humanization of the chicken-derived antibody was performed using the “CDR grafting” approach, a method originally described by Jones et al., Nature 321(6069): 522-5 (1986). First, the variable heavy (VH) and variable light (VL) regions of the antibodies were blasted against human IgG databases in order to find the closest human germline genes. This identified the IGHV3-23*01 (M99660) and human IGLV3-19*01 (X56178) genes as being closest to the chicken VH and VL genes, respectively. Similarly, the selected human amino acid sequences for J-gene region humanization were derived from IGHJ1*01 (J00256) and IGLJ6*01 (M18338) for VH and VL, respectively. The antibody VH and VL genes were then aligned against chicken immunoglobulin germline genes to identify somatic mutations in the framework regions that may play a role in antibody function and/or structure. Finally, certain amino acid positions, so-called “Vernier residues” (Nishibor et al., Mol Immunol. 43(6): 634-42 (2006), that are known to play an important role in antibody structure, stability and function, were considered for generating alternative humanized antibody variants including either human or chicken residues from the corresponding germlines.

Assembly of the chicken CDR and human framework regions was performed in silico and synthetic genes encoding humanized VH and VL were ordered from Genscript Inc. The VH and VL genes were cloned in expression vectors (plasmids) harboring the constant regions of human antibody light chain and heavy chain. Specifically, the VL was joined to the human lambda constant IGLC1*01 (J00252). In order to increase correct cleavage of the signal peptide upstream of the lambda chain, the second amino acid (Ser) of the Lambda gene IGLV3.19 was replaced by another amino acid (Tyr) which is present in other human germlines, for example IGLV3.25.

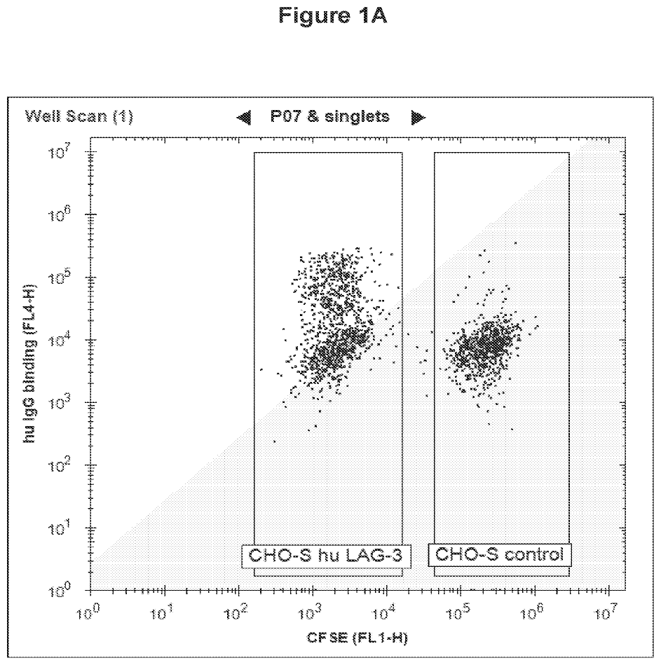

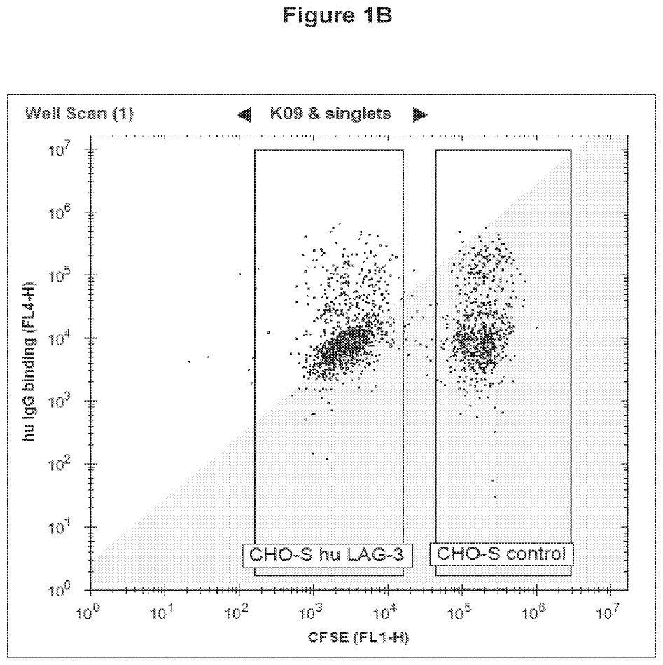

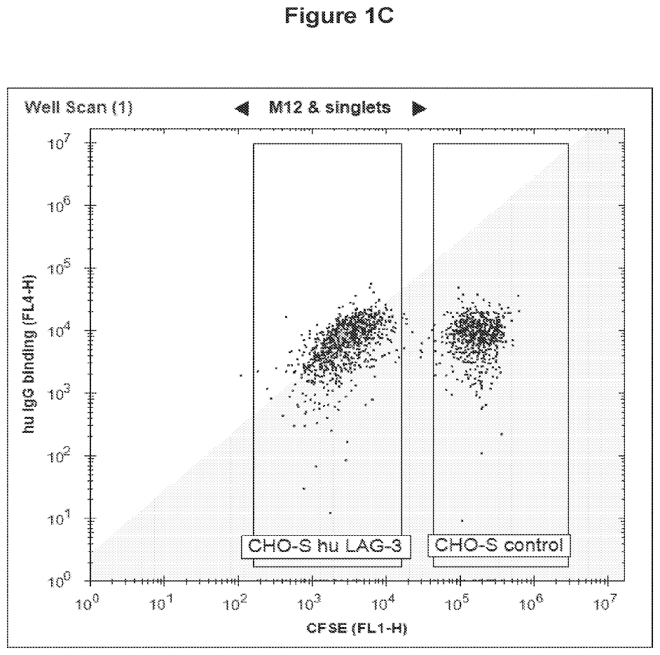

Representative flow cytometry dot plots for antibody clones generated as described in this example are shown in FIGS. 1A-C: (A) an antibody clone binding specifically to human LAG-3-transfected cells, (B) an antibody clone binding non-specifically to CHO-S cells, and (C) an antibody clone that does not bind to any of the cell populations used in the screening

### Example 3: Cloning of Anti-LAG-3 Reference Antibody Analogues

Amino acid sequences encoding the heavy and light chain variable domains of antibody analogues of 25F7-Lag3.5 and BAP050 were obtained from U.S. Patent Application Publication 2014/0093511 A1 (SEQ ID NOs: 12 and 14) and PCT Patent Application Publication WO 2015/138920 A1 (SEQ ID NOs: 6 and 16), respectively. The protein sequences were reverse translated to DNA sequences with human codon usage. The corresponding DNA sequences were then synthesized and cloned into expression vectors comprising a coding sequence for human IgG₄ heavy chain or kappa light chain constant domain, resulting in expression of full-length antibodies. To prevent Fab arm exchange, the serine residue at position 228 was substituted with proline (Angal et al., Mol. Immunol. 30: 105-108 (1993)). CHO cells were transfected with the resulting expression plasmids using a standard protein expression system. The corresponding antibody supernatants were purified using standard protein A purification column chromatography.

### Example 4: Screening of a Panel of LAG-3 Specific mAbs in the SEB+PBMC Assay

The ability of a large panel of LAG-3 specific mAbs to stimulate IL-2 secretion from Staphylococcal enterotoxin B (SEB) treated peripheral blood mononuclear cells (PBMCs) was evaluated using PBMCs from a single donor. SEB is a super-antigen that binds to MHC class II molecules and specific Vβ regions of T cell receptors (TCR) and drives non-specific stimulation of T-cells. This results in polyclonal T cell activation/proliferation and release of cytokines, including IL-2. Human PBMCs isolated from buffy coats from healthy donors were seeded in 384 well plates and left untreated or treated with 10 ng/ml SEB and 10 μg/mL of the antibodies. After 48 hours in a humidified incubator at 37° C., supernatants were removed and analyzed for IL-2 levels using an IL-2 ELISA kit (Life Technologies).

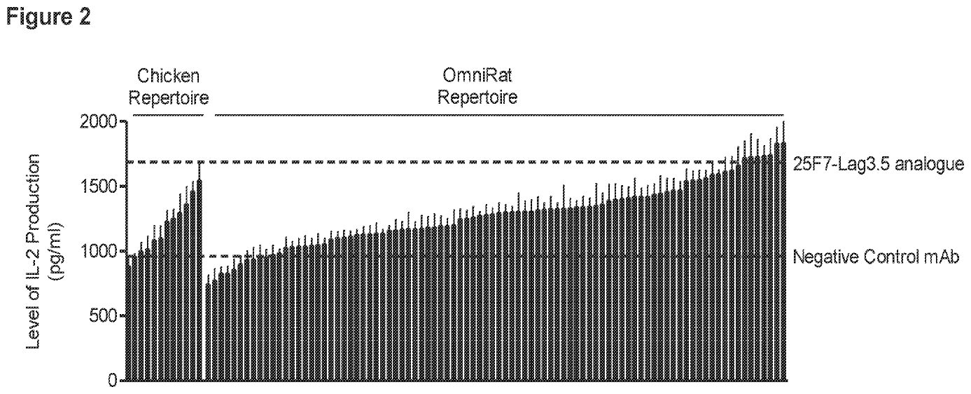

The increase in IL-2 secretion after treatment with the anti-LAG-3 mAb repertoires or the 25F7-Lag3.5 analogue is seen in FIG. 2, It is evident that the IL-2 levels after treatment with the different anti-LAG-3 mAbs varied strongly. showing that some antibodies in the repertoire had no functionality in this assay, whereas other antibodies induced IL-2 secretion to similar levels as the 25F7-Lag3.5 analogue.

### Example 5: Antibody Binding to Cell-Expressed Human or Cynomolgus LAG-3

To determine EC50 values for binding of antibodies by flow cytometry, CHO-S cells were transiently transfected to express human or cynomolgus LAG-3 using the FreeStyle™ MAX reagent (Invitrogen, Cat. No. 16447-100). Antibodies were titrated in 3-fold dilutions down from 10 to 0.05 μg/ml in staining buffer (PBS; 2% FBS; NaN₃) and mixed and incubated with either the human or cynomolgus LAG-3-transfected cells. After two washes in staining buffer, cell-bound antibody was revealed by addition of goat anti-human IgG (H+L) AF647 secondary antibody (Molecular Probes, Cat. No. A21445). Samples were obtained using high throughput flow cytometry (iQue® Screener, Intellicyt) and data was analyzed using ForeCyt® software by plotting antibody concentration vs. mean fluorescence intensity (MFI) for AF647. EC50 values were calculated using the “dose response graph” feature in ForeCyt®.

A summary of antibody binding properties measured by titration is found in Table 1 below, where huLAG-3 and cyLAG-3 are human and cynomolgus LAG-3-transfected cells, respectively. As shown in Table 1, antibodies 15646, 15532, 15723, 15595, 15431, and 15572 bind to both human and cynomolgus LAG-3.

TABLE 1
Antibody properties overview

| | | EC50 by FACS − | EC50 by FACS − |
|---|---|---|---|
| Antibody | Source | huLAG-3 (nM) | cyLAG-3 (nM) |
| 15646 | OmniRat ® | 0.10 | 0,11 |
| 15532 | OmniRat ® | 0.07 | 0.05 |
| 15723 | OmniRat ® | 0.09 | 0.26 |
| 15595 | OmniRat ® | 0.05 | 0.03 |
| 15431 | OmniRat ® | 0.04 | 0.04 |
| 15572 | OmniRat ® | 0.07 | 0.06 |
| 15011 | chicken | NA | NA |

### Example 6: Flow Cytometric Analysis of anti-LAG-3 Antibodies for MHCII Blocking Activity

This example illustrates how the anti-LAG-3 antibodies were tested for major histocompatibility complex II (MHCII) blocking activity by performing a flow cytometric competition assay using surface-MHCII-positive A375 cells and fluorochrome-labeled soluble LAG-3.

MHCII blocking activity was investigated in a cell-based assay using the human melanoma cell line A375 (ATCC® CRL-1619™), R-PE-labeled human LAG-3-Fc chimera protein can specifically bind to surface-expressed MHCII on A375, allowing quantification of this interaction by flow cytometry. Commercially available recombinant human LAG-3-Fc chimera protein (R&D Systems, USA) was conjugated to R-PE using the Lightning-Link# R-Phycoerythrin Conjugation Kit (Innova Biosciences, UK). A375 cells were harvested using enzyme-free Cell Dissociation Buffer (Gibco™), washed, and re-suspended in cold staining buffer (PBS, 2% FBS, NaN₃). Anti-LAG-3 antibodies to be tested were plated in 96-well format and adjusted to 20 μg/mL in 50 μL staining buffer. 1 μL of LAG-3-Fc-PE (corresponding to approx. 0.17 μg LAG-3-Fc) was added per well, mixed, and plates were incubated at 4° C. for 30 mins to allow LAG-3-antibody complex formation. During incubation, 1×10⁵ A375 cells (in 100 μL staining buffer) were plated in 96-well format, sedimented by centrifugation, and cell pellets were re-suspended in the pre-incubated LAG-3/antibody mixtures. Cells were incubated for additional 20 mins at 4° C., washed 1× in 200 μL cold staining buffer, and re-suspended in 100 μL staining buffer for acquisition.

Of the seven anti-LAG-3 antibodies tested (at 20 μg/mL), four, 15532, 15595, 15431 and 15011, induced a reduction of LAG-3 binding to MHCII (MFI) by approximately 90% compared to binding in the presence of a negative control antibody, and are thus considered effective blockers of the interaction. The remaining three antibodies. 15646, 15723 and 15572, only had limited effect on LAG-3-MHCII-binding and may be considered poor blockers. The reference antibody 25F7-Lag3.5 analogue displayed intermediate blocking activity. The results are summarized in Table 2.

TABLE 2
Blocking of LAG-3 binding to MHCII on A375 cells in the presence of
anti-LAG-3 antibodies.

| Antibody | LAG-3/MHCII Blocking (%)* |
|---|---|
| 15646 | 49 |
| 15532 | 89 |
| 15723 | 55 |
| 15595 | 94 |
| 15431 | 92 |
| 15572 | 37 |
| 15011 | 92 |
| 25F7-Lag3.5 analogue | 77 |

Median, normalized to LAG-3 binding in the presence of a negative control antibody.

### Example 7: Efficacy of Anti-LAG-3 Monoclonal Antibodies in SEB+PBMC Assay

The ability of the seven anti-LAG-3 mAbs to stimulate IL-2 secretion from Staphylococcal enterotoxin B (SEB) treated peripheral blood mononuclear cells (PBMCs) was evaluated. This example describes the efficacy of the seven anti-LAG-3 mAbs in several PBMC donors. In addition, the efficacy of four anti-LAG-3 mAbs from other sequence clusters is described. Sequence cluster numbers are shown in brackets after the antibody numbers and are further described in Example 13. The anti-LAG-3 antibodies were tested at 10 or 12.5 μg/ml in a SEB+PBMC assay as described in Example 4.

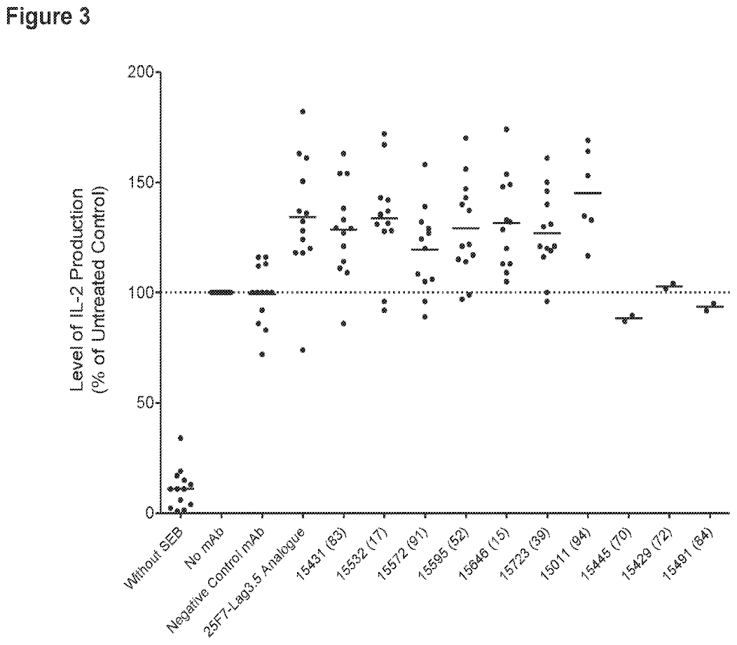

The increase in IL-2 secretion after treatment with the anti-LAG-3 mAbs 15431, 15532, 15572, 15595, 15646, 15723, and 15011 or the 25F7-Lag3.5 analogue is shown in FIG. 3. Each dot represents the level of IL-2 secretion from a single PBMC donor. It is evident that the IL-2 levels after treatment with the anti-LAG-3 mAbs varied among the different PBMC donors, but all seven anti-LAG-3 mAbs stimulated IL-2 production to similar levels. This is in contrast to the anti-LAG-3 mAbs 15445, 15429 and 15491, which do not increase IL-2 production.

### Example 8: Effect of anti-LAG-3 Monoclonal Antibodies in Cell-Based LAG-3-MHC Class II Blocking Assay

To characterize the seven anti-LAG-3 mAbs further, the ability to block the LAG˜ 3-MHC class II interaction was tested in a cell based assay.

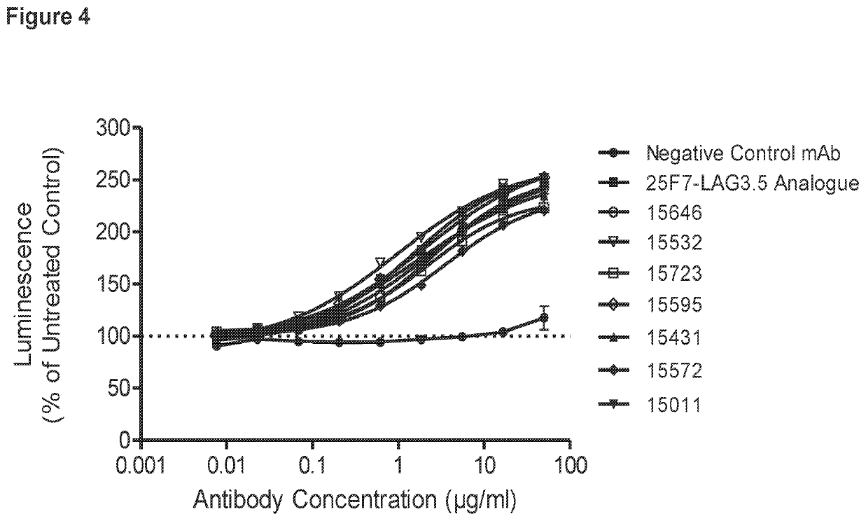

Jurkat cells harboring an NFAT-RE-controlled luciferase gene and expressing a modified TCR and LAG-3 (Promega) were incubated with the MHC class II-expressing Raji cell line, 50 ng/ml Staphylococcal enterotoxin D (SED) and different concentrations of monoclonal antibodies as indicated in FIG. 4. After 6 hours in a humidified incubator at 37° ° C., luciferase substrate (Promega) was added and luminescence was measured. Luminescence is a measure of luciferase expression, which is controlled by TCR signaling and inhibited by LAG-3 signaling. Thus, less LAG-3 signaling, e.g., by blocking the LAG-3-MHCII interaction, will lead to increased luminescence.

The increase in luminescence after treatment with the anti-LAG-3 mAbs 15431, 15532, 15572, 15595, 15646, 15723, and 15011 or the 25F7-Lag3.5 analogue is shown in FIG. 4. All of the tested anti-LAG-3 antibodies were found to increase the luminescence to similar levels.

### Example 9: Effect of Anti-LAG-3 Monoclonal Antibodies on Cellular Levels of LAG-3

To characterize selected anti-LAG-3 mAbs further, the ability to down-modulate LAG-3 levels was tested in a T-cell line overexpressing LAG-3.

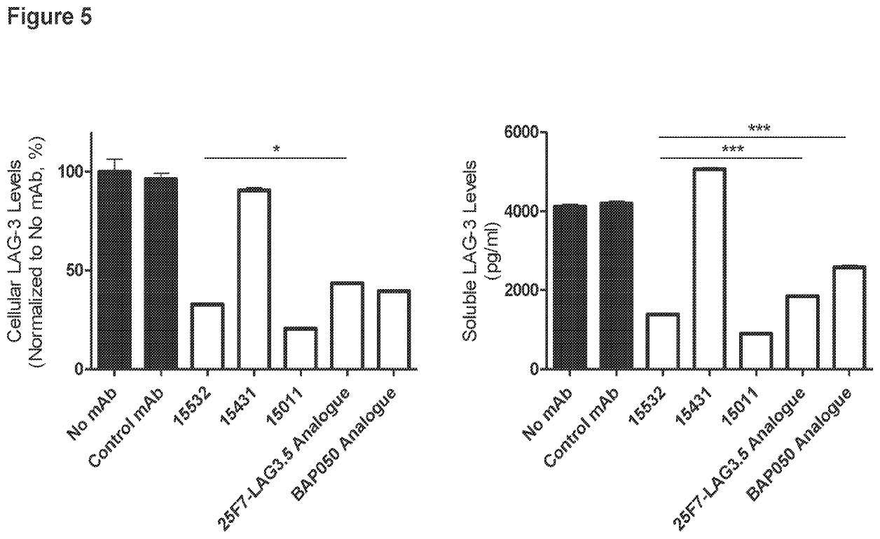

Jurkat cells expressing a modified TCR and LAG-3 (Promega) were incubated with 25 mg/mL of monoclonal antibodies as indicated in FIG. 5. After 24 hours in a humidified incubator at 37° C., superatants were collected and cell-lysed. Levels of LAG-3 in cells were evaluated with an anti-LAG-3 detection antibody (Novus Biologicals) and simple Westem technique (Sally Sue, Proteinsimple). Levels of LAG-3 in the supematant were evaluated using an anti-LAG-3 ELISA (R&D Systems). Binding competition studies confirmed that the tested anti-LAG-3 antibodies do not compete with the ELISA detection antibody for binding to soluble LAG-3. Statistical analysis was performed using one-way ANOVA with correction for multiple comparisons followed by a Bonferroni corrected, post-hoc t-test.

The levels of cellular and soluble LAG-3 after treatment with the anti-LAG-3 mAbs 15431, 15532, 15011, 25F7-Lag3.5 analogue or BAP050 analogue are shown in FIG. 5. 15532, 15011, 25F7-Lag3.5 analogue and BAP050 analogue decreased cellular as well as soluble LAG-3 levels. 15532 decreased cellular LAG-3 levels to a lower level than 25F7-Lag3.5 analogue with statistical significance, and decreased soluble LAG-3 levels to a lower level than 25F7-Lag3.5 analogue and BAP050 analogue with statistical significance. 15431 did not lower either cellular or soluble LAG-3 levels.

### Example 10: In Vivo Efficacy of Antibody 15011 in Two Syngeneic Murine Tumor Models

This example demonstrates the in vivo efficacy of antibody 15011 in two syngeneic murine tumor models.

### Methods

2×10⁵ Sa1N (fibrosarcoma) and 5×10⁶ ASB-XIV (lung carcinoma) cells were inoculated subcutaneously into the flank of 6-8 week old female A/J (Sa1N) or BALB/cAnNRj (ASB-XIV) mice. Tumors were measured three times weekly by caliper in two dimensions and tumor volume in mm3 was calculated according to the formula: (width)²×length×0.5. On day 5-7 post-inoculation, at an average tumor size of 30-50 mm³, the mice were randomized into two groups of ten animals and treatment initiated. The mice were treated three times weekly with a total of six treatments by intraperitoneal injection of vehicle buffer or the monoclonal antibody 15011 followed by an observation period. The antibody treatments were dosed at 10 mg/kg. Two-way ANOVA with Bonferroni's multiple comparisons test was applied to compare tumor volumes at each time-point between treatment groups. Statistical analyses were performed using GraphPad Prism version 5.0 (GraphPad Software, Inc.).

### Results

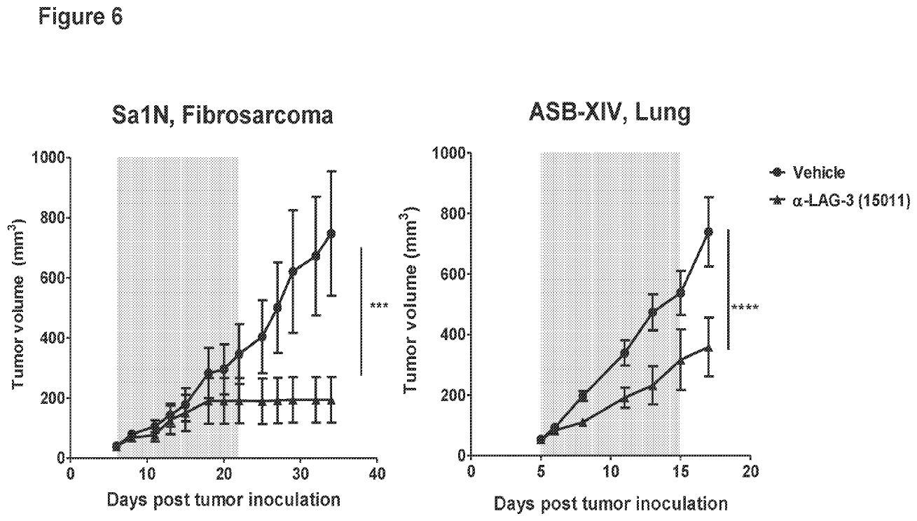

The results showed a profound tumor inhibitory effect of antibody 15011 in the tested syngeneic tumor models (*P<0.001) (FIG. 6). 15011 induced tumor growth regression in 50% of the mice engrafted with Sa1N tumors and resulted in tumor growth delay in the ASB-XIV syngeneic tumor model

### Example 11: In Vivo Efficacy of Antibody 15532 in NOG Mice Reconstituted With Human PBMC and Engrafted With Human Melanoma A375 Cells

This example demonstrates the in vivo efficacy of antibody 15532 in a semi-humanized xenograft tumor model, where the immune system in NOG mice was reconstituted with human PBMC and the mice engrafted with human melanoma A375 cells.

### Methods

On study day 0, NOG mice were subcutaneously injected with 2-4.5×10⁸ A375 melanoma cell and received 9 or 12×10⁶ PBMCs intraperitoneally on study day 2. PBMCs from one donor were used in each experiment. Treatment was initiated at the day of PBMC inoculation and the mice were treated three times weekly with a total of six or nine treatments by intraperitoneal injection of vehicle buffer, or the monoclonal antibody 15532 (10 mg/kg), followed by an observation period. Tumors were measured three times weekly by caliper in two dimensions and tumor volume in mm3 was calculated according to the formula: (width)²×length ×0.5. Two-way ANOVA with Bonferroni's multiple comparisons test was applied to compare tumor volumes at each time-point between treatment groups. Statistical analyses were performed using GraphPad Prism version 5.0 (GraphPad Software, Inc.).

### Results

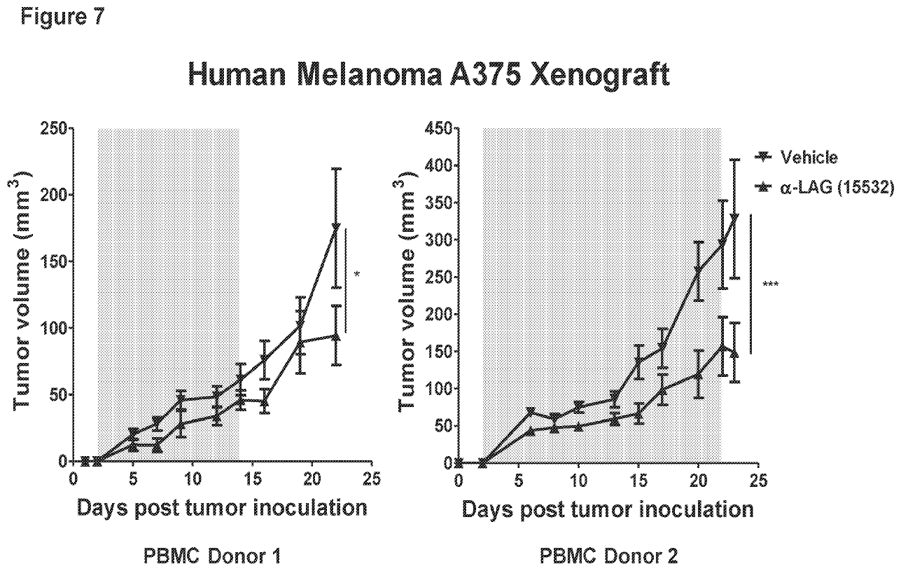

FIG. 7 shows results from two experiments using two different human PBMC donors that responded to anti-LAG-3 treatment. Treatment with 15532 resulted in significant tumor growth delay compared to the vehicle treated group [P<0.05 (donor 1) and P<0.001 (donor 2)].

### Example 12: Measurement of anti-LAG-3 Monoclonal Antibody Affinities Towards Human, Cynomolgus and Mouse LAG-3

This example demonstrates the binding of anti-LAG-3 antibodies towards human, cynomolgus and mouse LAG-3 extracellular domains as measured by Surface Plasmon Resonance (SPR). The protein sequence of human LAG-3 is available under UniProt Accession No. P18627 (SEQ ID NO: 68). The protein sequence of cynomolgus LAG-3 is available under NCBI Accession No. XP_005570011.1 (SEQ ID NO: 69). The protein sequence of mouse LAG-3 is available under UniProt Accession No. Q61790 (SEQ ID NO: 72).

### Materials and Methods

The kinetic binding analysis was performed by Surface Plasmon Resonance (SPR), using a Continuous Flow Microspotter (CFM, Wasatch Microfluidics, Salt Lake City, US) combined with an IBIS MX96 SPR instrument (IBIS Technologies, The Netherlands).

Anti-LAG-3 Fab antibodies were generated by digesting IgG₁ antibodies with GingisKHAN enzyme, using a kit provided by Genovis (Sweden).

The LAG-3 cDNAs coding for the extracellular domains of human and cynomolgus LAG-3 were synthesized and each cloned into a vector containing a CMV promoter and a human IgG₁ Fc sequence (AA P101-K330), resulting in fusion of IgG₁ Fc C-terminally to the cloned LAG-3 ECD. The LAG-3 Fc fusion constructs were generated by standard PCR and engineering techniques and protein was expressed transiently in 2 mL culture using an ExpiCHO™ expression system. The human LAG-3 Fc fusion constructs were harvested after 9 days and supernatants were tested for binding affinity to LAG-3 Fab antibodies by SPR. Antigens were purified using standard procedures and captured onto an G-a-hu-IgG Fc SensEye® (Ssens BV, The Netherlands) for 15 minutes using a Continuous Flow Microspotter (CFM, Wasatch Microfluidics, Salt Lake City, US). After spotting, the SensEye® was positioned in the IBIS MX96 biosensor and remaining capture sites blocked. Kinetic analysis was performed by applying a so called kinetic titration series (Karlsson R. 2006), where monomeric Fab fragments of the antibodies of the invention were injected in increasing concentrations from 1 nM to 1000 nM without application of surface regeneration steps after each Fab injection. Fab association was performed for 15 minutes and antigen dissociation was performed for 15 minutes. The recorded binding responses were fitted to a simple Langmuir 1:1 binding model with Scrubber 2 software for calculation of the on-rate (kon or ka), off-rate (koff or kd) and affinity (K_{D}) constants.

### Results

The results of the affinity measurement demonstrate that the evaluated antibodies 15532, 15431, 15572, 15011 and reference antibody analogue 25F7-Lag3.5 bind human and cynomolgus LAG-3 with different affinities. 15011 is the only antibody with mouse LAG-3 cross-reactivity. The detailed binding kinetics are tabulated in Table 3 below.

TABLE 3
Binding kinetics of anti-LAG-3 Fab fragments to human, cynomolgus
and mouse LAG-3 ECD as measured by SPR.

| Antibody | LAG-3 ECD | Kₒₙ (M-1 s-1) | K_{off} (s-1) | K_{D} (M) |
|---|---|---|---|---|
| 15532 | Human | 1.1E+05 | 6.5E−04 | 5.8E−09 |
| 15532 | Cynomolgus | 7.1E+04 | 4.8E−03 | 6.8E−08 |
| 15532 | Mouse | N.B. | N.B. | N.B. |
| 15431 | Human | 6.6E+04 | 2.0E−03 | 3.0E−08 |
| 15431 | Cynomolgus | 5.9E+04 | 6.8E−03 | 1.2E−07 |
| 15431 | Mouse | N.B. | N.B. | N.B. |
| 15572 | Human | 1.1E+05 | 3.0E−03 | 2.7E−08 |
| 15572 | Cynomolgus | 4.0E+04 | 4.0E−03 | 1.0E−07 |
| 15572 | Mouse | N.B. | N.B. | N.B. |
| 15011 | Human | 1.1E+05 | 4.8E−04 | 4.3E−09 |
| 15011 | Cynomolgus | 7.0E+04 | 2.5E−03 | 3.5E−08 |
| 15011 | Mouse | 7.2E+04 | 2.3E−03 | 3.3E−08 |
| 25F7-Lag3.5 | Human | 1.4E+05 | 1.2E−03 | 8.5E−09 |
| 25F7-Lag3.5 | Cynomolgus | 4.2E+03 | 3.8E−03 | 9.1E−07 |
| 25F7-Lag3.5 | Mouse | N.B. | N.B. | N.B. |

*NB: No binding

### Example 13: Epitope Binning of anti-LAG-3 Antibodies

This example illustrates how the anti-LAG-3 antibodies were grouped into epitope bins based on paired competition patterns in a sandwich assay. Antibodies belonging to different epitope bins recognize different epitopes on the LAG-3 ECD.

### Materials and Methods

Investigation of paired antibody competition was performed by Surface Plasmon Resonance (SPR) analysis using a Continuous Flow Microspotter (CFM) (Wasatch Microfluidics, US) combined with an IBIS MX96 SPR instrument (IBIS Technologies, The Netherlands). Surface Plasmon Resonance imaging analysis was performed on a goat-anti-human-IgG Fc SensEye® SPR sensor (Ssens BV, The Netherlands). A total of thirteen anti-LAG-3 antibodies, including reference antibody 25F7-Lag3.5, were diluted to 7.5 μg/mL in PBS buffer containing 0.05% Tween 20 (PBS-T), pH 7.0. Antibodies were captured onto the anti-Fc sensor surface by spotting for 15 minutes using a Continuous Flow Microspotter. After spotting, the SensEye® was positioned in the IBIS MX96 biosensor and residual anti-Fc sites blocked by injection of 30 μg/mL non-specific human IgG₁. Captured antibodies were conjugated to the surface using a FixIt kit (Ssens BV, The Netherlands). After sensor preparation, the immobilized antibodies (ligands) were used to capture monovalent LAG-3 antigen (50 nM, Acro Biosystems, China) in solution when injected over the sensor. Next, the panel of LAG-3 antibodies, diluted to 15 μg/mL in PBS-T buffer, were injected as analytes one by one over the sensor and tested for binding to captured LAG-3 in a sandwich assay to establish antibody competition patterns. After each antibody injection, the sensor surface was regenerated with 100 mM H₃PO₄ buffer, pH 3.0.

### Results

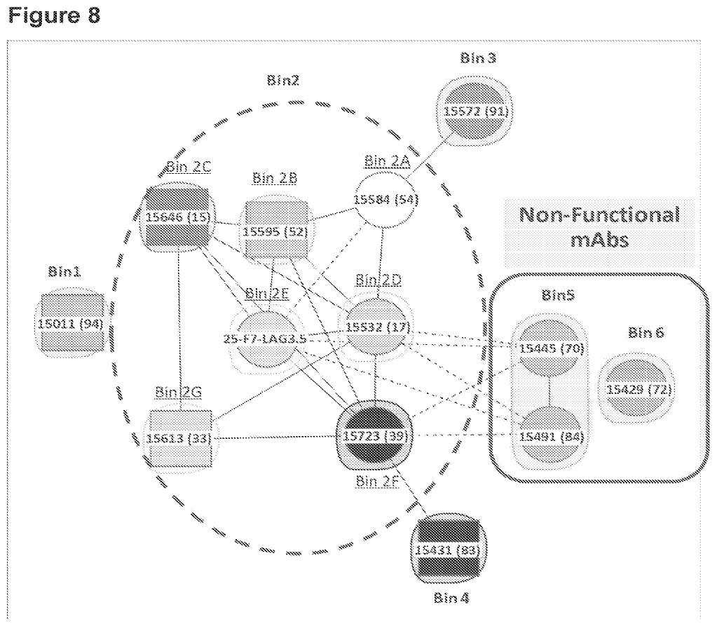

The competition pattern of the thirteen anti-LAG-3 antibodies is presented in FIG. 8. Antibodies are named according to their ID number and cluster group is listed in parenthesis. MAbs from identical cluster groups are expected to bind identical epitope bins, since the protein sequences from cluster members are highly related and only differ at few positions.

All antibodies were successfully epitope binned; however, antibodies 15595 (52). 15613 (33) and 15431 (38) did not work when used as ligands, and could only be tested as analytes. Antibodies 15646 (15) and 15011 (94) were only tested as ligands,

The competition analysis indicated that the panel of LAG-3 antibodies covers 6 major epitope bins (FIG. 8). Antibody 15011 (94) bound to a unique epitope that was not cross blocked by any other antibodies from the panel, and this antibody was consequently assigned to epitope bin 1. Epitope bin 2 contains seven antibodies that cross competed with some of the other mAbs from the group, and these antibodies were further divided into sub bins. MAb 15584 (54) was characterized by cross blocking both 15532 (17) and 15595 (52) when tested as a ligand or analyte, but it was not blocked by reference mAb 25F7-Lag3.5 when tested as an analyte, MAb 15613 (33) was only tested as an analyte and was shown to be blocked by 15723 (39), 15532 (17) and 15646 (15) but not 25F7-Lag3.5. Consequently, although 15532 (17) bound an epitope overlapping with 25F7-Lag3.5, the epitope of 15532 (17) is different from that of 25F7-Lag3.5 due to differences in competition with 15584 (84) and 15613 (33), and these antibodies were consequently assigned to different sub bins. Likewise, other mAbs in epitope bin 2 were assigned to sub bins based on competition patterns with other antibodies in bin 2 (FIG. 8).

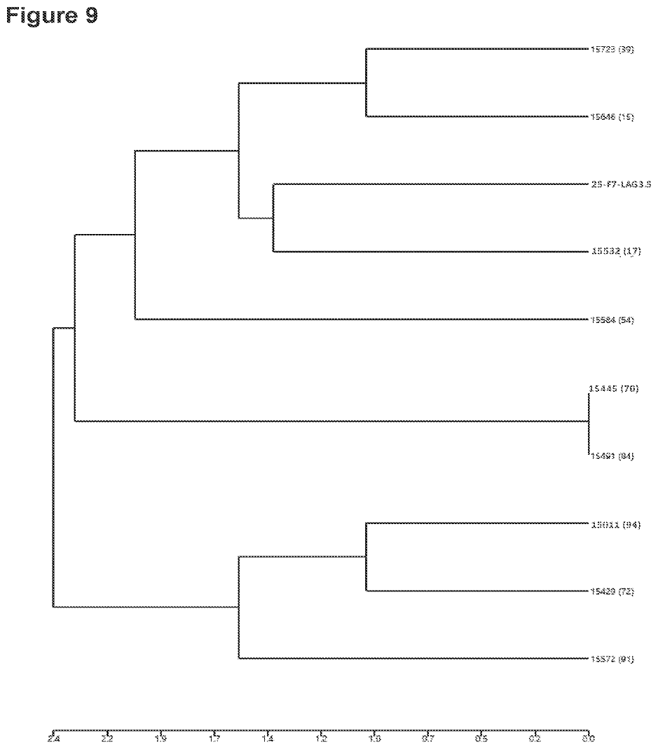

MAb 15572 (91) was only found to cross compete with mAb 15584 (54) and was assigned to a separate bin 3. Likewise, mAb 15431 (83) was assigned to a separate bin 4, because this antibody only cross competed with mAb 15723 (39). Finally, two separate epitope bins containing antibodies with nonfunctional activity were identified. Epitope bin 5 contained cross blocking antibodies 15445 (70) and 15491 (84) and epitope bin 6 contained mAb 15429 (72) that did not cross block any of the antibodies from the panel. Bin 5 antibodies also blocked antibodies 25F7-Lag3.5, 15532 (17) and 15723 (39) from bin 2, when these were tested as analytes (FIG. 8). A summary of the competition patterns for ligand antibodies is shown in FIG. 9, where the epitope relatedness of mAbs is shown based on clustering using Euclidean distance. From the cluster diagram it can be observed that antibodies with branching points at x-axis values above zero have different epitopes compared to other antibodies in the tested LAG-3 panel. MAbs 15595 (52), 15613 (33) and 15431 (38) were not included in the cluster analysis, because they did not work as ligands.

### Example 14: Epitope Mapping of LAG-3 Antibodies by LAG-3 Mutagenesis

Antibody epitopes can generally be characterized as linear epitopes (also termed continuous epitopes) or conformational epitopes (also termed discontinuous epitopes). While linear epitopes are defined based on a single continuous amino acid sequence. conformational epitopes may consist of many smaller discontinuous linear sequences or single contact residues. A collection of contact residues that cluster at the intermolecular protein interface between the antibody and the antigen is also termed a hot spot (Moreira et al., Proteins 68(4): 803-12 (2007). It is now widely acknowledged that most B-cell epitopes are discontinuous in nature (Sivalingam and Shepherd, Mol Immunol. 51(3-4): 304-92012 (2012), Kringelum et al., Mol Immunol. 53(1-2): 24-34 (2013)) with the average epitope spanning 15-22 amino acid residues of which 2-5 amino acids contribute to most of the binding (Sivalingam and Shepherd, supra).

By ranking binding affinity to 108 different LAG-3 mutants, this example illustrates how the binding epitopes of the anti-LAG-3 antibodies 15532, 15431, 15572, and 15011 can be divided into linear epitopes and hotspots that are distinct from the epitope recognized by the reference antibody 25F7-Lag3.5 analogue.

### Methods

Human LAG-3 receptor (CD223) is a 525 amino acid (AA) transmembrane protein that comprises an extracellular domain (ECD) of 427 amino acids (residues 23-450) followed by a transmembrane domain (residues 451-471) and a cytoplasmic domain (residues 472-525). LAG-3 ECD contains 4 immunoglobulin domains (D1-D4) where the first domain, an IgV-type domain, is involved in ligand binding (MHCII binding) and the remaining three domains are IgC2 type domains. The first two N-terminal domains alone (D1-D2) are sufficient for ligand binding, and D2 has been shown to be required for correct receptor presentation (Huard et al., Proc Natl Acad Sci USA 94: 5744-5749 (1997), Andrews et al., Immunol Rev. 276(1): 80-96 (2017)). Domain 1 contains an additional unique stretch of 30 amino acids (extra loop) that is not found in other IgV-domains and that has been shown to be essential for ligand binding. Amino acids (AA) 99, 110, 125, 131 and 137 have also been identified as important for ligand binding (Y77, R88, R103, D109, R115 in the mature protein) (Huard et al, supra; Andrews et al., supra). There is no published structure for LAG-3. However, sequence and evolutionary homology between LAG-3 and CD4 have been identified, thus allowing access to structural information on LAG-3 based on homology modeling of CD4 (Huard et al., supra; Andrews et al., supra).

The protein sequences of human LAG-3 and orthologues were downloaded from UniProt or NCBI: human (P18627; SEQ ID NO: 68), cynomolgus monkey (Macaca fascicularis, XP_005570011.1; SEQ ID NO: 69), rat (Rattus norvegicus, Q5BK54; SEQ ID NO: 70) and dog (Canis Lupus Familiaris, F1P7Z3; SEQ ID NO: 71). The sequence identities of the different LAG-3 extracellular domain amino acid sequences are shown in Table 4 below.

TABLE 4
List of amino acid differences and sequence identity to human LAG-3
in the extracellular domain of LAG-3 from different species.

| | Amino Acid Differences | % Sequence Identity |
|---|---|---|
| Cynomolgus LAG-3 ECD | 34 | 92.1 |
| Rat LAG-3 ECD | 140 | 67.3 |
| Dog LAG-3 ECD | 197 | 54.1 |

To map linear epitopes in the context of the native human LAG-3 structure, 35 chimeric proteins were designed, where 10 amino acids in the human LAG-3 ECD sequence were sequentially exchanged to rat sequence in segments that overlapped by 5 amino acids, and supplemented with dog and cynomolgus versions in regions of particular interest (e.g. critical AA insertion sequences and loop stretches). Sequence exchanges were performed in domain 1 of human LAG-3 spanning amino acids 23-170. From a constructed homology model based on CD4 crystal structures (1WIO. 1WIQ) and amino acid sequence alignments, surface exposed amino acids were identified and 80 individual substitutions were designed on human LAG-3 domain 1. Introduced point mutations were primarily alanine substitutions. When the surface exposed residue was alanine, this position was changed to serine.

LAG-3 cDNA coding for domains 1 and 2 of the human LAG-3 extracellular domain (AA 1-266) was gene synthesized and cloned into a vector containing CMV promoter and human IgG₁ Fc sequence (AA P101-K330), resulting in expression of a LAG domain 1-2 Fc fusion protein. The mutated human LAG-3 Fc fusion constructs were generated by standard PCR and engineering techniques and protein was expressed transiently in 2 ml culture using an ExpiCHO™ expression system. The human LAG-3 Fc fusion constructs were harvested, purified and tested for binding affinity to anti-LAG-3 mAbs by Surface Plasmon Resonance (SPR). The LAG-3 fusion proteins were immobilized onto a G-a-hu-IgG Fc SensEye® (Ssens BV, The Netherlands) for 15 minutes using a Continuous Flow Microspotter (CFM, Wasatch Microfluidics, Salt Lake City, US). After spotting, the SensEye® was positioned in an IBIS MX96 biosensor and captured proteins were evaluated for binding affinity. Kinetic analysis was performed by applying a so called kinetic titration series (Karlsson R. 2006), where monomeric Fab fragments of the antibodies of the invention were injected in increasing concentrations from 0.4 nM to 300 nM without application of surface regeneration steps after each Fab injection. Fab association was performed for 15 minutes and antigen dissociation was performed for 15 minutes. The recorded binding responses were fitted to a simple Langmuir 1:1 binding model with Scrubber 2 software for calculation of the on-rate (kon or ka), off-rate (koff or kd) and affinity (KD) constants.

### Results

The binding affinities of Fab fragments of anti-LAG-3 antibodies 15532, 15431, 15572, 15011 and the reference antibody 25F7-Lag3.5 analogue were evaluated with respect to altered binding to LAG-3 mutant constructs The binding affinities of Fab fragments binding to the mutated LAG-3 constructs were expressed as the ratio between KD mutant/KD wildtype (normalized binding affinity). Tables 5 and 6 below show the normalized binding affinities to all tested chimeric proteins and for the alanine scanning experiments, respectively. A cut-off of at least 5-fold affinity reduction was employed as a criterion for detecting significantly reduced binding affinity to mutated LAG-3 constructs. In some instances, no binding could be detected to specific antibodies. These constructs are listed as not binding (N.B.).

The analysis showed that the binding epitopes of anti-LAG-3 antibodies 15532, 15431, 15572, 15011 are distinct from that of the reference antibody 25F7-Lag3.5 analogue. The binding epitope of 15532 was evident from the chimeric proteins with AA inserted at positions 78-87, 84-92, 88-97—a collective amino acid stretch in LAG-3 that is within the domain 1 extra loop. However, unlike 25F7-Lag3.5, the linear epitopes of 15532 extend beyond this stretch, including AA 95-100, AA 98-105 and AA123-131. Interestingly, key amino acids for ligand interactions have been identified in the segment from 99-131 (Y99, R125 & D131: Huard et al, supra). The 25F7-Lag3.5 analogue did not show any sensitivity for mutations in the region critical for ligand binding. The 15532 contact residues as measured by alanine substitutions were identified as H85, P86, A87, P89, S91, W92 and G93 whereas the 25F7-Lag3.5 analogue did not show sensitivity towards P86 and S91, illustrating differentiated contact residues within the AA 85-93 region. 15532 was able to bind chimeric construct #28 with cynomolgus AA backmutations (P84H; H85R; S90Y), whereas the 25F7-Lag3.5 analogue was not, again reflecting a differentiated fine epitope within the AA 84-90 region.

The two antibodies 15431 and 15572 were both found to bind linear epitopes at segments AA 23-30 and 40-66. Further alanine scanning illustrated that both antibodies shared identical contact residues at positions A40, Q41, P43, P46, P49, D52, T62, Q64, H65, Q66, P67 and D68. 15431 was additionally sensitive to chimeric constructs 88-97, 95-100 and 98-105 (segment AA 88-105). The AA region 98-105 proved to be a unique shared epitope between 15532, 15431, 15572 and 15011. The 25F7-Lag3.5 analogue did not bind this epitope. Alanine scanning indicated that 15431 and 15572 shared contact residues P96, Y99, T100, V101, P106 and G107, while 15431 had unique contacts at positions G93, P94, and R98, The antibody 15431 also had a linear epitope in segments AA 123-131, 128-137 and 148-154. Interestingly, positions 125, 131 and 137 have previously been shown to be important for ligand binding (Huard et al, supra). These segments (AA 123-131, 128-137 and 148-154) were also important for 15572, which recognized linear epitopes in the segments AA 118-137 and 148-161. Alanine scanning further showed that both 15431 and 15572 shared contact residues at positions R119, E124, R129, G130, D131, S133, R137, P138, D143, R148 and R163 while the 25F7-Lag3.5 analogue did not show sensitivity towards any of these epitope regions or amino acid positions.

Antibody 15011 contained a linear epitope defined by a chimeric construct with mutations in the range from AA 98-105, and alanine scanning further extended the unique epitope to include contact residues at positions G107, L109, R110 and S111. Interestingly, R110 has been shown to be important for ligand binding (Huard et al, supra). The amino acid sequence is well conserved in this region (AA 98-113) between human, cynomolgus, mouse and rat orthologues, explaining the cross-reactivity of this antibody to mouse and cynomolgus monkey LAG-3 (Example 12). A summary of the collected epitope mapping findings is shown in Table 7 below.

In summary, we have shown at single amino acid resolution, by analyzing binding to a panel of 108 LAG-3 mutants, that the four antibodies 15532, 15431, 15572 and 15011 recognize unique but partially overlapping epitopes in domain 1 of LAG-3. This finding is consistent with the epitope binning analysis of Example 13, showing that 15532 cross-competes with the 25F7-Lag3.5 analogue, but has a differentiated competition pattern with other LAG-3 antibodies and hence a different epitope (Table 7). 15431, 15572 and 15011 did not compete for binding with 15532 or 25F7-Lag3.5 and had unique competition patterns with discrete epitope bin 2 antibodies. The assigned epitope bins (Example 13) match well the epitope mapping that revealed that each antibody showed unique differentiated epitopes (Table 7). The epitope data including chimeric construct #28 also corroborated well with the binding affinity analysis (Example 12) in which 15532 showed significant binding to cynomolgus LAG-3 and 25F7-Lag3.5 did not—reflecting different properties of the antibodies due to differentiated contact residues.

TABLE 5
Summary of binding affinity analysis for Fab fragments of anti-LAG-3 antibodies binding mutated LAG-3 ECD
constructs with inserted rat. dog or cynomolgus sequence segments. Normalized binding expressed as K_{D} mutant/K_{D}
wild type.

| Chimeric | Scanned | Mutated | | | | | | |
|---|---|---|---|---|---|---|---|---|
| construct | region | region | | | | | | 25F7-Lag3.5 |
| # | hu LAG-3 | hu LAG-3 | Introduced mutations from other species | 15532 | 15431 | 15572 | 15011 | analogue |
| 1 | AA 23-32 | AA 23-30 | L23S; Q24G; A27K; V29L; P30S | 3.2 | N.B. | N.B. | 0.5 | 1.3 |
| 2 | AA 28-37 | AA 29-30 | V29L; P30S | 3.1 | N.B. | N.B. | 0.5 | 1.2 |
| 3 | AA 33-42 | AA 40-41 | A40V; Q41H | 2.2 | N.B. | N.B. | 0.5 | 1.3 |
| 4 | AA 38-47 | AA 40-47 | A40V; Q41H; P46L; T47E | 3.9 | N.B. | N.B. | 0.5 | 1.3 |
| 5 | AA 43-52 | AA 46-51 | P46L; T47E; I48F; L50H; Q51L | 2.7 | N.B. | N.B. | 0.5 | 1.3 |
| 6 | AA 48-57 | AA 48-55 | I4BF; L50H; Q51L; L53P; S54N; L55F | 3.4 | N.B. | N.B. | 0.5 | 1.4 |
| 7 | AA 53-62 | AA 53-60 | LS3P; S54N; LS5F; A59G; G60W | 3.4 | N.B. | N.B. | 0.5 | 1.4 |
| 8 | AA 58-67 | AA 59-66 | AS9G; G60W; Q66R | 2.7 | N.B. | N.B. | 0.5 | 13 |
| 9 | AA 63-72 | AA 66-71 | Q66R: G70D; P71Q | 3.9 | 0.4 | 1.1 | 0.7 | 1.3 |
| 10 | AA 68-77 | AA 70-77 | G700; Q71Q; A74S; A75I; G77A | 1.1 | 1.1 | 0.7 | 1.8 | 1.0 |
| 11 | AA 73-82 | AA 74-82 | A745; A75I; G77A; H78L; P79D; AB1L; P82Q | 1.5 | 0.4 | 0.5 | 0.6 | 1.1 |
| 12 | AA 78-87 | AA 78-87 | H78L; P79D; A81L; P82Q; delP84-A87 | N.B. | 0.8 | 0.8 | 0.9 | N.B. |
| 13 | AA 83-92 | AA 84-92 | delP84-A87; A88M; S91T; W92R | N.B. | 0.6 | 1.1 | 1.3 | N.B. |
| 14 | AA 88-97 | AA 88-97 | AB8M; S91T; W92R; G93R; P94H; R95P; R97H | N.B. | N.B. | 0.7 | 0.6 | N.B. |
| 15 | AA 93-102 | AA 95-100 | R95G; R98S; T100V | 6.5 | N.B. | 3.1 | 1.9 | 1.5 |
| 16 | AA 98-107 | AA 98-105 | R98S; T100V; S103M; V104R; G105A | 6.3 | N.B. | N.B. | 7.2 | 1.7 |
| 17 | AA 108-117 | AA 114-117 | L114Q; Q117L | 1.4 | 0.5 | 0.5 | 2.6 | 1.3 |
| 18 | AA 113-122 | AA 114-119 | L114Q; Q117L; P118S; R119H | 1.1 | 0.6 | 0.6 | 2.1 | 0.8 |
| 19 | AA 118-127 | AA 118-127 | P118S; R119H; D123E; E124K; R127P | 0.5 | 0.5 | N.B. | 0.6 | 0.8 |
| 20 | AA 123-132 | AA 123-131 | D123N; E124S; R125P; R127P; Q128T; delG130-D131 | 5.4 | N.B. | N.B. | 0.3 | 1.3 |
| 21 | AA 128-137 | AA 128-137 | Q128T; delG130-D131; W135A; R137Q | 4.7 | N.B. | N.B. | 0.4 | 1.4 |
| 22 | AA 133-142 | AA 140-142 | R140T; A142K | 1.3 | 0.6 | 0.8 | 0.5 | 1.2 |
| 23 | AA 143-152 | AA 148-152 | R148H; A150F; H152R | 2.5 | 3.5 | N.B. | 2.0 | 0.8 |
| 24 | AA 148-156 | AA 148-154 | R148H; A150F; H152R; R154P | 2.6 | 10.6 | N.B. | 1.9 | 0.7 |
| 25 | AA 153-160 | AA 154-158 | R154P; A157D; L158F | 1.1 | 0.8 | N.B. | 1.9 | 0.7 |
| 26 | AA 157-165 | AA 157-161 | A157D; L158F; R161S | 1.1 | 0.6 | N.B. | 0.7 | 0.9 |
| 27 | AA 161-170 | AA 161-166 | R161S; L166V | 0.8 | 1.3 | 1.7 | 2.3 | 0.8 |
| 28 | AA 83-92 | AA 84-90 | P84H; H85R; S90Y | 4.8 | 1.1 | 0.5 | 0.6 | N.B. |

| | |
|---|---|
| | <5-fold KD change |
| | Chimeric mutants. |
| bold | >5-fold KD change |
| | Chimeric mutants |
| N.B. | No binding of |
| | Chimeric mutants |

TABLE 6
Summary of binding affinity analysis for Fab fragments of anti-LAG-3
antibodies binding point mutation scanned human LAG-3. Normalized
binding expressed as K_{D} mutant/K_{D} wild type.

| Point | Mutated | | | | | 25F7- |
|---|---|---|---|---|---|---|
| mutation | AA in | | | | | Lag3.5 |
| # | hu LAG-3 | 15532 | 15431 | 15572 | 15011 | analogue |
| 1 | A40S | 4.4 | N.B. | N.B. | 0.6 | 1.1 |
| 2 | Q41A | 3.4 | N.B. | N.B. | 0.7 | 1.0 |
| 3 | P43A | 3.8 | N.B. | N.B. | 0.6 | 1.2 |
| 4 | S4SA | 1.7 | 0.4 | 0.6 | 0.7 | 1.0 |
| 5 | P46A | 4.0 | N.B. | N.B. | 0.7 | 1.1 |
| 6 | T47A | 1.5 | 0.9 | 0.6 | 0.9 | 0.8 |
| 7 | P49A | 1.7 | N.B. | N.B. | 0.8 | 1.1 |
| 8 | QS1A | 0.9 | 0.7 | 0.6 | 0.8 | 0.8 |
| 9 | DSZA | 1.7 | N.B. | N.B. | 0.7 | 0.8 |
| 10 | S54A | 0.7 | 0.5 | 0.7 | 0.8 | 0.8 |
| 11 | L56A | 1.2 | 0.3 | 0.7 | 0.7 | 0.9 |
| 12 | R57A | 1.1 | 0.6 | 0.8 | 0.7 | 0.7 |
| 13 | R58A | 0.8 | 1.2 | 0.8 | 1.7 | 0.8 |
| 14 | T62A | 4.2 | N.B. | N.B. | 0.6 | 1.3 |
| 15 | Q64A | 2.9 | N.B. | N.B. | 0.6 | 1.3 |
| 16 | H65A | 3.8 | N.B. | N.B. | 0.5 | 1.2 |
| 17 | Q66A | 2.7 | N.B. | N.B. | 0.7 | 0.9 |
| 18 | P67A | 3.8 | N.B. | N.B. | 0.6 | 1.1 |
| 19 | D68A | 3.4 | N.B. | N.B. | 0.6 | 0.9 |
| 20 | A73S | 0.7 | 0.6 | 0.6 | 0.7 | 0.8 |
| 21 | A74S | 1.0 | 0.8 | 0.7 | 0.8 | 0.9 |
| 22 | A75S | 0.9 | 0.8 | 0.7 | 0.8 | 0.9 |
| 23 | P76A | 1.0 | 0.7 | 0.6 | 0.8 | 0.9 |
| 24 | G77A | 1.0 | 0.7 | 0.7 | 0.8 | 1.0 |
| 25 | H78A | 0.9 | 0.4 | 0.5 | 0.7 | 0.8 |
| 26 | P79A | 0.9 | 0.4 | 0.4 | 0.6 | 0.8 |
| 27 | L80A | 0.6 | 0.5 | 0.6 | 0.8 | 0.7 |
| 28 | A81S | 0.6 | 0.6 | 0.6 | 0.8 | 0.7 |
| 29 | PB2A | 1.0 | 1.0 | 0.8 | 0.9 | 0.9 |
| 30 | G83A | 1.1 | 0.5 | 0.5 | 0.7 | 1.0 |
| 31 | P84A | 0.5 | 1.1 | 0.9 | 1.0 | 0.6 |
| 32 | HBSA | 7.2 | 0.8 | 0.7 | 0.9 | 14.1 |
| 33 | P86A | 6.1 | 0.6 | 0.7 | 0.7 | 2.7 |
| 34 | A87S | N.B. | 1.0 | 0.9 | 10 | N.B. |
| 35 | A8BS | 3.7 | 0.6 | 0.6 | 0.9 | 3.5 |
| 36 | P89A | 14.2 | 0.8 | 0.8 | 0.9 | N.B. |
| 37 | S90A | 0.6 | 0.9 | 0.8 | 0.9 | 1.0 |
| 38 | S91A | 5.3 | 0.7 | 0.7 | 0.8 | 2.2 |
| 39 | W92A | N.B. | 3.6 | 1.0 | 1.1 | N.B. |
| 40 | G93A | 7.1 | 11.6 | 0.8 | 1.0 | 6.2 |
| 41 | P94A | 0.8 | N.B. | 0.6 | 0.7 | 0.8 |
| 42 | R95A | 3.7 | 1.9 | 0.6 | 0.7 | 1.9 |
| 43 | P96A | 1.4 | N.B. | N.8. | 0.6. | 0.7 |
| 44 | R97A | 0.9 | 1.3 | 0.6 | 0.7 | 0.9 |
| 45 | R98A | 1.6 | N.B. | 0.9 | 1.2 | 0.9 |
| 46 | Y99A | 4.1 | N.B. | N.B. | 0.5 | 1.0 |
| 47 | T100A | 3.9 | N.B. | N.B. | 0.6 | 1.2 |
| 48 | V101A | 3.5 | N.B. | N.8. | 0.5 | 1.0 |
| 49 | G10SA | 1.1 | 0.4 | 0.4 | 1.6 | 1.0 |
| 50 | P106A | 2.7 | N.B. | N.B. | 4.6 | 1.0 |
| 51 | G107A | 2.1 | N.B. | N.B. | 5.7 | 0.8 |
| 52 | G108A | 1.0 | 0.3 | 0.4 | 0.5 | 0.9 |
| 53 | L109A | 1.1 | 1.0 | 1.0 | 84.2 | 0.9 |
| 54 | R110A | 0.9 | 0.9 | 0.6 | N.B. | 0.9 |
| 55 | S111A | 1.0 | 0.8 | 0.7 | 53.4 | 0.9 |
| S6 | G112A | 1.0 | 0.5 | 0.5 | 3.1 | 0.9 |
| 57 | R113A | 0.6 | 0.9 | 0.7 | 3.3 | 0.8 |
| 58 | P11SA | 1.0 | 1.1 | 1.4 | 1.3 | 1.0 |
| 59 | Q117A | 0.8 | 0.4 | 0.5 | 0.7 | 0.8 |
| 60 | P118A | 0.7 | 1.0 | 0.9 | 1.2 | 0.8 |
| 61 | R119A | 3.2 | N.B. | N.B. | 0.6 | 1.1 |
| 62 | Q121A | 0.9 | 1.0 | 0.6 | 0.9 | 0.9 |
| 63 | D123A | 1.0 | 0.9 | 0.5 | 0.8 | 1.0 |
| 64 | E124A | 1.8 | N.B. | N.B. | 0.8 | 1.1 |
| 65 | R125A | 0.9 | 1.1 | 1.1 | 1.1 | 0.9 |
| 66 | G126A | 0.9 | 0.9 | 0.9 | 0.9 | 0.9 |
| 67 | R127A | 0.6 | 0.9 | 0.5 | 0.9 | 0.7 |
| 68 | Q128A | 0.7 | 0.8 | 0.9 | 1.0 | 0.7 |
| 69 | R129A | 3.0 | N.B. | N.B. | 0.7 | 1.2 |
| 70 | G130A | 4.4 | N.B. | N.B. | 0.6 | 1.3 |
| 71 | D131A | 4.2 | N.B. | N.B. | 0.6 | 1.2 |
| 72 | S133A | 2.2 | N.B. | N.B. | 0.6 | 1.1 |
| 73 | R137A | 3.6 | N.B. | N.B. | 0.6 | 1.0 |
| 74 | P138A | 2.7 | N.B. | N.B. | 0.7 | 1.0 |
| 75 | R141A | 0.7 | 1.0 | 0.9 | 1.1 | 0.8 |
| 76 | D143A | 3.8 | N.B. | N.B. | 0.5 | 1.0 |
| 77 | R148A | 2.9 | N.B. | N.B. | 0.7 | 1.2 |
| 78 | R154A | 0.8 | 1.0 | 0.8 | 1.2 | 0.8 |
| 79 | S159A | 0.7 | 0.9 | 0.3 | 0.7 | 0.8 |
| 80 | R163A | 2.5 | N.B. | N.B. | 0.6 | 1.0 |

| | |
|---|---|
| | <5-fold KD change |
| | Point mutants |
| bold | >5-fold KD change |
| | Point mutants |
| N.B. | No binding of |
| | Point mutants |

TABLE 7
Summary of the binding epitopes identified for tested anti-LAG-3 antibodies

| Antibody | Epitope Bin | Linear epitopes | Contact Residues |
|---|---|---|---|
| 15532 | 2D | 78-105 | H85, P86, A87, P89, S91, W92, G93 |
| | | 123-131 | |
| 15431 | 4 | 23-30 | A40, Q41, P43, P46, P49, D52 |
| | | 40-66 | T62, Q64, H65, Q66, P67, 068 |
| | | 88-105 | G93, P94, P96, R98, Y99, T100, V101, P106, G107 |
| | | 123-137 | R119, E124 |
| | | 148-152 | R129, G130, D131, S133, R137, P138, |
| | | | D143, R148, R163 |
| 15572 | 3 | 23-30 | A40, Q41, P43, P46, P49, D52 |
| | | 40-66 | T62, Q64, H65, Q66, P67, D68 |
| | | 98-105 | P96, Y99, T100, V101, P106, G107 |
| | | 118-137 | R119, E124 |
| | | 148-161 | R129, G130, D131, S133, R137, P138, |
| | | | D143, R148, R163 |
| 15011 | 1 | 98-105 | G107, L109, R110, S111 |
| 25F7-Lag3.5 | 2E | 78-97 | H85, A87, P89, W92, G93 |
| analogue | | | |

TABLE 8
SEQ ID NOs for VH, VL, HC and LC DNA and protein sequences

| SEQ | |
|---|---|
| ID NO | Sequence |
| 1 | 15646 heavy chain variable domain (VH) DNA sequence |
| 2 | 15646 light chain variable domain (VL) DNA sequence |
| 3 | 15646 VH protein sequence |
| 4 | 15646 VL protein sequence |
| 5 | 15532 VH DNA sequence |
| 6 | 15532 VL DNA sequence |
| 7 | 15532 VH protein sequence |
| 8 | 15532 VL protein sequence |
| 9 | 15723 VH DNA sequence |
| 10 | 15723 VL DNA sequence |
| 11 | 15723 VH protein sequence |
| 12 | 15723 VL protein sequence |
| 13 | 15595 VH DNA sequence |
| 14 | 15595 VL DNA sequence |
| 15 | 15595 VH protein sequence |
| 16 | 15595 VL protein sequence |
| 17 | 15431 VH DNA sequence |
| 18 | 15431 VL DNA sequence |
| 19 | 15431 VH protein sequence |
| 20 | 15431 VL protein sequence |
| 21 | 15572 VH DNA sequence |
| 22 | 15572 VL DNA sequence |
| 23 | 15572 VH protein sequence |
| 24 | 15572 VL protein sequence |
| 25 | 15011 VH DNA sequence |
| 26 | 15011 VL DNA sequence |
| 27 | 15011 VH protein sequence |
| 28 | 15011 VL protein sequence |
| 29 | IgG₁-LALA heavy chain (HC) constant region DNA sequence |
| 30 | IgG₁-LALA heavy chain (HC) constant region protein sequence |
| 31 | Ig lambda light chain (LC) constant region DNA sequence |
| 32 | Ig lambda light chain (LC) constant region protein sequence |
| 33 | Ig kappa light chain (KC) constant region DNA sequence |
| 34 | Ig kappa light chain (KC) constant region protein sequence |

TABLE 9
SEQ ID NOs for the VH and VL DNA and protein sequences and the H-CDR1-3 and L-CDR1-3 amino acid
sequences of the anti-LAG-3 antibodies

| Antibody | VH (DNA) | VL (DNA) | VH (Protein) | VL (Protein) | H-CDR1 | H-CDR2 | H-CDR3 | L-CDR1 | L-CDR2 | L-CDR3 |
|---|---|---|---|---|---|---|---|---|---|---|
| 15646 | 1 | 2 | 3 | 4 | 35 | 36 | 37 | 38 | 39 | 40 |
| 15532 | 5 | 6 | 7 | 8 | 41 | 42 | 43 | 44 | 45 | 40 |
| 15723 | 9 | 10 | 11 | 12 | 35 | 42 | 46 | 44 | 47 | 40 |
| 15595 | 13 | 14 | 15 | 16 | 48 | 49 | 50 | 51 | 47 | 52 |
| 15431 | 17 | 18 | 19 | 20 | 53 | 54 | 55 | 44 | 45 | 40 |
| 15572 | 21 | 22 | 23 | 24 | 56 | 57 | 58 | 59 | 60 | 61 |
| 15011 | 25 | 26 | 27 | 28 | 62 | 63 | 64 | 65 | 66 | 67 |

TABLE 10
Complementarity determining regions (CDRs) of anti-LAG-3 antibodies and germline
gene origin of OMT rat-derived antibodies.

| Antibody | | | | | |
|---|---|---|---|---|---|
| number | IGHV gene | H-CDR1 | H-CDR2 | H-CDR3 | SEQ ID NOS |
| 15646 | IGHV4-34 | GGSFSGYY | INHRGST | CTRGEEWESLFFDYW | 35-37 |
| 15532 | IGHV4-34 | GESFSGYY | INHSGST | CARGWDLLDWNDYWNEYW | 41-43 |
| 15723 | IGHV4-34 | GGSFSGYY | INHSGST | CARGEDWGESFFDYW | 35, 42, 46 |
| 15595 | IGHV1-24 | GYSLTEIS | FDPEDGET | CATGGWGPNWFDPW | 48-50 |
| 15431 | IGHV6-1 | GDSVSSNSA | TYYRSKW | CARDDDWNDFDYW | 53-55 |
| 15572 | IGHV4-39 | GDSISSSSYY | IFYSGNT | CAREDDFLTDYYGAFDIW | 56-58 |
| 15011 | IGHV3-23* | GFDERSYA | INGEVGGSNT | CVKGAGACGICNDDIDAW | 62-64 |
| Antibody | | | | | |
| number | IGKV gene | L-CDR1 | L-CDR2 | L-CDR3 | SEQ ID NOS |
| 15646 | IGKV3-11 | QSISSY | GAS | CQQRSNWPLTF | 38-40 |
| 15532 | IGKV3-11 | QSVSSY | DAS | CQQRSNWPLTF | 44, 45, 40 |
| 15723 | IGKV3-11 | QSVSSY | AAS | CQQRSNWPLTF | 44, 47, 40 |
| 15595 | IGKV1-12 | QGISSW | AAS | CQQANSEPFTF | 51, 47, 52 |
| 15431 | IGKV3-11 | QSVSSY | DAS | CQQRSNWPLTF | 44, 45, 40 |
| 15572 | IGKV1-5 | QSISSW | KAS | CQQYNSYLTF | 59-61 |
| 15011 | IGLV3-19* | GSYAGSY | DND | CGSTNDNDDGGLF | 65-67 |

*Genes used for humanization, see Example 2.

Sequence List

VH and VL DNA and protein sequences
SEQ ID NO: 1 (15646 VH DNA sequence)
CAGGTGCAGCTGCAGCAGTGGGGTGCCGGTCTGCTGAAGCCTTCTGAAACTCTGTCTCTGACTT
GTGCCGTCTATGGTGGATCATTCAGCGGCTACTATTGGTCCTGGATCAGGCAGCCCCCTGGCAA
GGGCCTGGAGTGGATCGGCGAGATCAACCACCGGGGCTCTACCAACTACAATCCCTCTCTGAAG
AGCAGGGTGACCATCTCCGTGGACACATCTAAGAATCAGTTCAGCCTGAAGCTGAGCTCCGTGA
CCGCCGCTGATACAGCCGTGTACTATTGCACAAGGGGGGAGGAATGGGAGTCACTGTTCTTTGA
TTACTGGGGGCAGGGGACACTGGTCACAGTCTCGAGT
SEQ ID NO: 2 (15646 VL DNA sequence)
GAAATCGTCCTGACCCAGTCCCCCGCCACCCTGAGCCTGAGCCCCGGAGAAAGAGCCACCCTGT
CCTGCCGAGCAAGCCAGTCCATCAGCTCCTATCTCGCCTGGTATCAGCAGAAACCAGGCCAGGC
TCCCCGGCTGCTGATCTACGGCGCCTCCAACAGAGCTACAGGAATCCCAGCCCGCTTCAGCGGC
TCCGGCTCTGGCACAGACTTTACCCTGACAATCTCTAGCCTGGAGCCTGAGGATTTCGCCGTGT
ACTATTGCCAGCAGAGATCTAATTGGCCACTGACATTCGGCGGCGGCACACGGGTGGAGATCAA
G
SEQ ID NO: 3 (15646 VH protein sequence)
QVQLQQWGAGLLKPSETLSLTCAVYGGSFSGYYWSWIRQPPGKGLEWIGEINHRGSTNYNPSLK
SRVTISVDTSKNQFSLKLSSVTAADTAVYYCTRGEEWESLFFDYWGQGTLVTVSS
SEQ ID NO: 4 (15646 VL protein sequence)
EIVLTQSPAILSLSPGERAILSCRASQSISSYLAWYQQKPGQAPRLLIYGASNRATGIPARFSG
SGSGTDFTLTISSLEPEDPAVYYCQQRSNWPLTFGGGTRVEIK
SEQ ID NO: 5 (15532 VH DNA sequence)
CAGGTTCAGCTGCAGCAGTGGGGCGCCGGCCTGCTGAGACCAAGCGAGACCCTGTCCCTGACAT
GCGCCGTGTATGGCGAGAGCTTCTCCGGCTATTACTGGAACTGGATCCGGCAGCCTCCCGGCAA
GGGCCTGGAGTGGATCGGCGAGATCAATCACTCCGGCTCCACCAATTACAACCCATCCCTGAAG
TCTCGGGTGACAATCAGCGTGGATACAAGCAAGACCCAGTTCAGCCTGAAGCTGAGCTCCGTGA
CAGCTGCCGATACCGCCGTGTATTACTGCGCCAGAGGCTGGGACCTGCTGGATTGGAATGACTA
CTGGAATGAGTACTGGGGCCAGGGGACCCTGGTGACCGTCTCGAGT
SEQ ID NO: 6 (15532 VL DNA sequence)
GAGATCGTGCTGACCCAGTCCCCTGCCACCCTGTCTCTGTCCCCTGGCGAGCGGGCGACCCTGT
CCTGTAGAGCTTCTCAGTCCGTGTCTTCCTACCTGGCTTGGTACCAGCAGAAGCCAGGACAGGC
CCCAAGACTGCTGATCTATGACGCTTCCAATCGGGCTACCGGCATCCCAGCTCGCTTTAGCGGC
TCCGGCTCCGGCACCGACTTCACCCTGACAATCAGCTCCCTGGAGCCAGAGGATTTTGCCGTGT
ATTACTGTCAGCAGAGGTCCAATTGGCCACTGACATTTGGCGGCGGCACAAAGGTTGAGATCAA
G
SEQ ID NO: 7 (15532 VH protein sequence)
QVQLQQWGAGLLRPSETLSLICAVYGESFSGYYWNWIRQPPGKGLEWIGEINHSGSTNYNPSLK
SRVTISVDTSKTQFSLKLSSVTAADTAVYYCARGWDLLDWNDYWNEYWGQGTLVTVSS
SEQ ID NO: 8 (15532 VL protein sequence)
EIVLTQSPATLSLSPGERAILSCRASQSVSSYLAWYQQKPGQAPRLLIYDASNRATGIPARFSG
SGSGTDFTLTISSLEPEDFAVYYCQQRSNWPLTFGGGTKVEIK
SEQ ID NO: 9 (15723 VH DNA sequence)
CAGGTTCAGCTGCAGCAGTGGGGCGCITGCCTGCTGAAGCCCTCTGAGACCCTGTCTCTGACCT
GTGCCGTGTATGGCGGCAGCTTCTCCGGCTATTACTGGAACTGGATCCGCCAGCCCCCCGGCAA
GGGCCTGGAGTGGATCGGCGAGATCAACCACTCCGGCTCTACCAACTACAATCCTTCTCTGAAG
TCCAGGGTGACAATCAGCGTGGACACCAGCAAGAACCAGTTTAGCCTGAAGCTGTCCAGCGTGA
CAGCTGCCGATACAGCCGTGTATTACTGCGCCAGAGGCGAGGATTGGGGCGAGAGCTTCTTTGA
TTACTGGGGCCAGGGGACCCTGGTGACCGTCTCGAGT
SEQ ID NO: 10 (15723 VL DNA sequence)
GAGATCGTGCTGACCCAGTCCCCTGCCACCCTGTCTCTGTCCCCTGGCGAGCGGGCCACCCTGA
GCTGTCGGGCCTCCCAGTCCGTGAGCTCCTACCTGGCTTGGTATCAGCAGAAGCCAGGACAGGC
CCCAAGACTGCTGATCTACGCCGCTTCCAATCGGGCCACCGGCATCCCCGCCAGATTTTCCGGC
TCTGGCTCCGGCACCGATTTCACCCTGACAATCAGCTCCCTGGAGCCAGAGGACTTCGCCGTGT
ACTACTGCCAGCAGAGGTCTAATTGGCCACTGACATTTGGGGGCGGCACCAAGGTTGAGATCAA
G
SEQ ID NO: 11 (15723 VH protein sequence)
QVQLQQWGAGLLKPSETLSLTCAVYGGSFSGYYWNWIRQPPGKGLEWIGEINHSGSTNYNPSLK
SRVTISVDTSKNQFSLKLSSVTAADTAVYYCARGEDWGESFFDYWGQGTLVTVSS
SEQ ID NO: 12 (15723 VL protein sequence)
EIVLTQSPATLSLSPGERATLSCRASQSVSSYLAWYQQKPGQAPRLLIYAASNRATGIPARFSG
SGSGTDFTLTISSLEPEDFAVYYCQQRSNWPLTFGGGTKVEIK
SEQ ID NO: 13 (15595 VH DNA sequence)
CAGGTTCAGCTGGTGCAGTCCGGCGCTGAGGTGAAGAAGCCTGGAGCTTCTGTGAAGGTTTCCT
GTAAGGTTTCCGGCTATAGCCTGACCGAGATCTCTATGCACTGGGTACGGCAAGCCCCCGGCAA
GGGCCTGGAGTGGATGGGCGGCTTTGACCCAGAGGATGGCGAGACCATCTACGCTCAGAGGTTT
CAGGGGCGCGTGATCATGACCGAGGATACCAGCACCGATACCGCCTACATGGAGCTGTCCAGCC
TGAGATCCGAGGATACCGCCGTGTATTACTGCGCTACTGGTGGCTGGGGCCCCAATTGGTTCGA
TCCTTGGGGCCAGGGGACCCTGGTGACCGTCTCGAGT
SEQ ID NO: 14 (15595 VL DNA sequence)
GACATCCAGATGACACAGTCTCCTTCCTCTGTGAGCGCCTCTGTGGGCGACCGCGTGACCATCA
CATGCCGCGCTTCCCAGGGGATCTCTTCCTGGCTGGCTTGGTACCAGCAGAAGCCCGGCAAGGC
CCCCAAGCTGCTGATCTATGCTGCCAGCAGCCTGCAGTCCGGCGTGCCTTCCAGGTTTAGCGGC
TCCGGCTCCGGCACCGACTTTACACTGACAATCAGCTCCCTGCAGCCCGAGGATTTTGCCACCT
ATTACTGTCAGCAGGCGAATTCCTTCCCTTTTACATTCGGCCCTGGCACCAAGGTTGATATCAA
G
SEQ ID NO: 15 (15595 VH protein sequence)
QVQLVQSGAEVKKPGASVKVSCKVSGYSLTEISMHWVRQAPGKGLEWMGGFDPEDGETTYAQRE
QGRVIMTEDTSTDTAYMELSSLRSEDTAVYYCATGGWGPNWFDPWGQGTLVTVSS
SEQ ID NO: 16 (15595 VL protein sequence)
DIQMTQSPSSVSASVGDRVTITCRASQGISSWLAWYQQKPGKAPKLLIYAASSLQSGVPSRFSG
SGSGTDFTLTISSLQPEDFATYYCQQANSFPFTFGPGTKVDIK
SEQ ID NO: 17 (15431 VH DNA sequence)
CAGGTTCAGCTGCAGCAGTCCGGCCCCGGCCTGGTGAAGCCAAGCCAGACCCTGTCTCTGACAT
GCGCCATCTCCGGCGACAGCGTGTCCTCTAACTCCGCCGCTTGGAATTGGATCCGGCAGTCTCC
ATCCAGAGGCCTGGAGTGGCTGGGCAGAACCTATTACCGGTCCAAGTGGTACAACGATTATGCC
GTGTCCGTGAAGAGCAGAATCACCATCAACCCTGATACCAGCAAGAACCAGTTCAGCCTGCAGC
TGAATTCCGTGACCCCAGAGGATACAGCCGTGTATTACTGTGCTAGGGACGATGATTGGAATGA
CTTCGATTACTGGGGCCAGGGGACCCTGGTGACAGTCTCGAGT
SEQ ID NO: 18 (15431 VL DNA sequence)
GAGATCGTGCTGACCCAGTCCCCAGCTACACTGTCCCTGTCTCCCGGGGAGCGGGCCACCCTGA
GCTGTAGAGCTTCCCAGTCCGTGTCTTCCTATCTGGCTTGGTATCAGCAGAAGCCAGGACAGGC
CCCAAGGCTGCTGATCTACGACGCCTCCAATAGAGCCACCGGCATCCCAGCTAGATTTTCTGGC
TCCGGCTCCGGCACCGATTTCACACTGACCATCTCTAGCCTGGAGCCAGAGGATTTTGCTGTAT
ATTACTGCCAGCAGCGCAGCAACTGGCCCCTGACATTTGGCGGCGGCACCAAGGTTGAGATCAA
G
SEQ ID NO: 19 (15431 VH protein sequence)
QVQLQQSGPGLVKPSQTLSLTCAISGDSVSSNSAAWNWIRQSPSRGLEWLGRTYYRSKWYNDYA
VSVKSRITINPDISKNQFSLQLNSVTPEDTAVYYCARDDDWNDFDYWGQGTLVTVSS
SEQ ID NO: 20 (15431 VL protein sequence)
EIVLTQSPATLSLSPGERATLSCRASQSVSSYLAWYQQKPGQAPRLLIYDASNRATGIPARFSG
SGSGTDFTLTISSLEPEDFAVYYCQQRSNWPLTFGGGTKVEIK
SEQ ID NO: 21 (15572 VH DNA sequence)
CAGCTGCAGCTGCAGGAAAGCGGCCCCGGCCTGGTGAAGCCCTCTGAGACCCTGTCCCTGACAT
GCACCGTGAGCGGCGATTCCATCAGCTCTTCCAGCTATTACTGGGGCTGGATCCGGCAGCCCCC
CGGCAAGGGCCTGGAGTGGATCGGCAGCATCTTCTACTCCGGCAATACATATTATAATCCTTCT
CTGAAGAGCAGGGTGACAATCAGCGTGGATACCTCCAAGAATCAGTTTAGCCTGAAGCTGAGCT
CCGTGACAGCTGCCGATACAGCCGTGTATTACTGCGCTAGGGAGGACGATTTTCTGACCGATTA
TTACGGCGCTTTCGACATCTGGGGCCAGGGGACAATGGTGACAGTCTCGAGT
SEQ ID NO: 22 (15572 VL DNA sequence)
GATATCCAGATGACCCAGTCTCCAAGCACCCTGAGCGCCTCTGTGGGCGATCGGGTGACCATCA
CATGTCGGGCTTCTCAGTCCATCAGCAGCTGGCTGGCTTGGTATCAGCAGAAGCCCGGCAAGGC
CCCAAAGCTGCTGATCTACAAGGCCTCTTCCAGCGAGAGCGGCGTGCCATCCAGGTTTAGCGGC
TCCGGCTCCGGCACCGAGTTTACCCTGACCATCTCTTCCCTGCAGCCCGATGACTTTGCCACCT
ACTACTGTCAGCAGTACAATTCCTATCTGACATTCGGCGGCGGCACCAAGGTTGAGATCAAG
SEQ ID NO: 23 (15572 VH protein sequence)
QLQLQESGPGLVKPSETLSLTCTVSGDSISSSSYYWGWIRQPPGKGLEWIGSIFYSGNTYYNPS
LKSRVTISVDTSKNQFSLKLSSVTAADTAVYYCAREDDFLTDYYGAFDINGQGTMVTVSS
SEQ ID NO: 24 (15572 VL protein sequence)
DIQMTQSPSTESASVGDRVTITCRASQSISSWLAWYQQKPGKAPKLLIYKASSSESGVPSRFSG
SGSGTEFTLTISSLQPDDFATYYCQQYNSYLTFGGGTKVEIK
SEQ ID NO: 25 (15011 VH DNA sequence)
GAGGTGCAGCTGCTGGAATCCGGAGGAGGACTGGTCCAGCCAGGTGGATCCCTGCGACTGAGCT
GCGCCGCTTCTGGCTTCGACTTTAGAAGCTACGCAATGATGTGGGTCCGCCAGGCACCAGGAAA
GGGACTGGAGTGGGTGGGAGGGATCAACGGTGAAGTCGGTGGCTCTAATACATACTATGCACCT
GCCGTCAAGGGAAGGGCTACTATTAGTCGGGACAACTCAAAAAATACCCTGTATCTGCAAATGA
ACAGTCTGAGGGCCGAGGATACCGCCGTGTACTATTGCGTGAAAGGTGCTGGCGCATGCGGCAT
CTGTAATGACGATATTGATGCATGGGGACAGGGGACCCTGGTGACAGTCTCGAGT
SEQ ID NO: 26 (15011 VL DNA sequence)
AGTTATGAGCTGACTCAGGACCCAGCAGTGTCAGTCGCCCTGGGCCAGACAGTGAGAATCACTT
GCAGTGGGGCTGGTTCATATGCAGGCTCCTACTATTACGGATGGCACCAGCAGAAGCCCGGACA
GGCACCTGTGACAGTCATCTACGACAACGATAAAAGGCCAAGCAATATTCCCGACCGGTTCTCT
GGGTCCAGCTCTGGTAACACCGCCTCCCTGACCATTACTGGGGCCCAGGCTGAGGACGAAGCTG
ATTATTACTGTGGCTCTACAAACGATAATGACGATGGCGGACTGTTTGGCTCCGGAACTAAGGT
CACCGTCCTA
SEQ ID NO: 27 (15011 VH protein sequence)
EVQLLESGGGLVQPGGSLRLSCAASGFDFRSYAMMWVRQAPGKGLEWVGGINGEVGGSNTYYAP
AVKGRATISRDNSKNTLYLQMNSLRAEDTAVYYCVKGAGACGICNDDIDAWGQGTLVTVSS
SEQ ID NO: 28 (15011 VL protein sequence)
SYELTQDPAVSVALGQTVRITCSGAGSYAGSYYYGWHQQKPGQAPVIVIYDNDKRPSNIPDRFS
GSSSGNTASLTITGAQAEDEADYYCGSTNDNDDGGLFGSGTKVTVL
Constant region DNA and protein sequences
SEQ ID NO: 29 (lgHC constant region DNA sequence)
GCCTCCACCAAGGGCCCATCGGTCTTCCCCCTGGCACCCTCCTCCAAGAGCACCTCTGGGGGCA
CAGCGGCCCTGGGCTGCCTGGTCAAGGACTACTTCCCCGAACCGGTGACGGTGTCGTGGAACTC
AGGCGCCCTGACCAGCGGCGTGCACACCTTCCCGGCTGTCCTACAGTCCTCAGGACTCTACTCC
CTCAGCAGCGTGGTGACCGTGCCCTCCAGCAGCTTGGGCACCCAGACCTACATCTGCAACGTGA
ATCACAAGCCCAGCAACACCAAGGTGGACAAGAGAGTTGAGCCCAAATCTTGTGACAAAACTCA
CACATGCCCACCGTGCCCAGCACCTGAAGCCGCCGGGGGACCGTCAGTCTTCCTCTTCCCCCCA
AAACCCAAGGACACCCTCATGATCTCCCGGACCCCTGAGGTCACATGCGTGGTGGTGGACGTGA
GCCACGAAGACCCTGAGGTCAAGTTCAACTGGTACGTGGACGGCGTGGAGGTGCATAATGCCAA
GACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTACACCCTGCC
CCCATCCCGGGAGGAGATGACCAAGAACCAGGTCAGCCTGACCTGCCTGGTCAAAGGCTTCTAT
CCCAGCGACATCGCCGTGGAGTGGGAGAGCAATGGGCAGCCGGAGAACAACTACAAGACCACGC
CTCCCGTGCTGGACTCCGACGGCTCCTTCTTCCTCTATAGCAAGCTCACCGTGGACAAGAGCAG
GTGGCAGCAGGGGAACGTCTTCTCATGCTCCGTGATGCATGAGGCTCTGCACAACCACTACACG
CAGAAGAGCCTCTCCCTGTCCCCGGGTAAA
SEQ ID NO: 30 (IgHC constant region protein sequence)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVKDRVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHEALHNHYT
QKSLSLSPGK
SEQ ID NO: 31 (IgLC constant region DNA sequence)
GGCCAGCCCAAGGCCAACCCCACTGTCACTCTGTTCCCGCCCTCCTCTGAGGAGCTCCAAGCCA
ACAAGGCCACACTAGTGTGTCTGATCAGTGACTTCTACCCGGGAGCTGTGACAGTGGCCTGGAA
GGCAGATGGCAGCCCCGTCAAGGCGGGAGTGGAGACCACCAAACCCTCCAAACAGAGCAACAAC
AAGTACGCGGCCAGCAGCTACCTGAGCCTGACGCCCGAGCAGTGGAAGTCCCACAGAAGCTACA
GCTGCCAGGTCACGCATGAAGGGAGCACCGTGGAGAAGACAGTGGCCCCTACAGAATGTTCA
SEQ ID NO: 32 (IgLC constant region protein sequence)
GQPKANPTVTLFPPSSEELQANKATLVCLISDFYPGAVTVANKADGSPVKAGVETTKPSKQSNN
KYAASSYLSLTPEQWKSHRSYSCQVTHEGSTVEKTVAPTECS
SEQ ID NO: 33 (IgKC constant region DNA sequence)
CGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACG
CCTGCGAAGTCACCCATCAGGGCCTGAGCTCGCCCGTCACAAAGAGCTTCAACAGGGGAGAGTG
T
SEQ ID NO: 34 (IgKC constant region protein sequence)
RTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSENRGEC
SEQ ID NO: 35 (15646/15723 H-CDR1)
GGSFSGYY
SEQ ID NO: 36 (15646 H-CDR2)
INHRGST
SEQ ID NO: 37 (15646 H-CDR3)
CTRGEEWESLFFDYW
SEQ ID NO: 38 (15646 L-CDR1)
QSISSY
SEQ ID NO: 39 (15646 L-CDR2)
GAS
SEQ ID NO: 40 (15646/15532/15723/15431 L-CDR3)
CQQRSNWPLTF
SEQ ID NO: 41 (15532 H-CDR1)
GESFSGYY
SEQ ID NO: 42 (15532/15723 H-CDR2)
INHSGST
SEQ ID NO: 43 (15532 H-CDR3)
CARGWDLLDWNDYWNEYW
SEQ ID NO: 44 (15532/15723/15431 L-CDR1)
QSVSSY
SEQ ID NO: 45 (15532/15431 L-CDR2)
DAS
SEQ ID NO: 46 (15723 H-CDR3)
CARGEDWGESFFDYW
SEQ ID NO: 47 (15723/15595 L-CDR2)
AAS
SEQ ID NO: 48 (15595 H-CDR1)
GYSLTEIS
SEQ ID NO: 49 (15595 H-CDR2)
FDPEDGET
SEQ ID NO: 50 (15595 H-CDR3)
CATGGWGPNWFDPW
SEQ ID NO: 51 (15595 L-CDR1)
QGISSW
SEQ ID NO: 52 (15595 L-CDR3)
CQQANSFPFTF
SEQ ID NO: 53 (15431 H-CDR1)
GDSVSSNSA
SEQ ID NO: 54 (15431 H-CDR2)
TYYRSKW
SEQ ID NO: 55 (15431 H-CDR3)
CARDDDWNDFDYW
SEQ ID NO: 56 (15572 H-CDR1)
GDSISSSSYY
SEQ ID NO: 57 (15572 H-CDR2)
IFYSGNT
SEQ ID NO: 58 (15572 H-CDR3)
CAREDDFLTDYYGAFDIW
SEQ ID NO: 59 (15572 L-CDR1)
QSISSW
SEQ ID NO: 60 (15572 L-CDR2)
KAS
SEQ ID NO: 61 (15572 L-CDR3)
CQQYNSYLTF
SEQ ID NO: 62 (15011 H-CDR1)
GFDFRSYA
SEQ ID NO: 63 (15011 H-CDR2)
INGEVGGSNT
SEQ ID NO: 64 (15011 H-CDR3)
CVKGAGACGICNDDIDAW
SEQ ID NO: 65 (15011 L-CDR1)
GSYAGSY
SEQ ID NO: 66 (15011 L-CDR2)
DND
SEQ ID NO: 67 (15011 L-CDR3)
CGSTNDNDDGGLF
SEQ ID NO: 68 (human LAG-3)
MWEAQFLGLLFLQPLWVAPVKPLQPGAEVPVVWAQEGAPAQLPCSPTIPLQDLSLLRRAG
VTWQHQPDSGPPAAAPGHPLAPGPHPAAPSSWGPRPRRYTVLSVGPGGERSGRLPLQPRV
QLDERGRQRGDFSLWLRPARRADAGEYRAAVHLRDRALSCRLRLRLGQASMTASPPGSLR
ASDWVILNCSFSRPDRPASVHWFRNRGQGRVPVRESPHHHLAESFLFLPQVSPMDSGPWG
CILTYRDGFNVSIMYNLTVLGLEPPTPLTVYAGAGSRVGLPCRLPAGVGTRSFLTAKWTP
PGGGPDLLVTGDNGDFTLRLEDVSQAQAGTYTCHIHLQEQQLNATVTLAIITVTPKSFGS
PGSLGKLLCEVTPVSGQERFVWSSLDTPSQRSFSGPWLEAQEAQLLSQPWQCQLYQGERL
LGAAVYFTELSSPGAQRSGRAPGALPAGHLLLFLILGVLSLLLLVTGAFGFHLWRRQWRP
RRFSALEQGIHPPQAQSKIEELEQEPEPEPEPEPEPEPEPEPEQL
SEQ ID NO: 69 (cynomolgus LAG-3)
MWEAQFLGLLFLOPLWVAPVKPPQPGAEISVVWAQEGAPAQLPCSPTIPLQDLSLLRRAGVTWQHQPDSG
PPAXAPGHPPVPGHRPAAPYSWGPRPRRYTVLSVGPGGLRSGRLPLQPRVQLDERGRQRGDFSLWLRPAR
RADAGEYRATVHLRDRALSCRLRLRVGQASMTASPPGSLRTSDWVILNCSFSRPDRPASVHWFRSRGQGR
VPVQGSPHHHLAESFLFLPHVGPMDSGLWGCILTYYDGFNVSIMYNLTVLGLEPATPLTVYAGAGSRVEL
PCRLPPAVGTQSFLTAKWAPPGGGPDLLVAGDNGDFTLRLEDVSQAQAGTYICHIRLQGQQLNATVTLAI
ITVTPKSFGSPGSLGKLLCEVTPASGQEHFVWSPLNTPSQRSFSGPWLEAQEAQLLSQPWQCQLHQGERL
LGAAVYFTELSSPGAQRSGRAPGALRAGHLPLFLILGVLFLLLLVTGAFGFHLWRRQWRPRRFSALEQGI
HPPQAQSKIEELEQEPELEPEPELERELGPEPEPGPEPEPEQL
SEQ ID NO: 70 (rat LAG-3)
MRQDLFLDLLLLQLIWEAPVVSSGPGKELSVVWAQEGAPVHLPCSLEFPHLDPNFLRRGW
VTWQHRPDSDQPASIPALDLLQGMPSTRRHPPHRYTVLSVAPGGLRSGROPLLSHVQLEK
RGPQRGDFSLWLRPATRKDAGEYHAFVRLPDRDFSCSLRLRVGQASMIASPPGTLKPSDW
VILNCSFSRPDRPVSVHWFQGQSRVPVHNSPRHYLAESFLLLPQVSPLDSGTWGCVLTYR
DGFNVSITYNLKVQGLEPVAPLTVYAAEGSRVELPCHLPPVVGTPSLLIAKWTPPGGGPE
LPVTGKSGNFTLQLENVGRAQAGTYTCSTHLQGRQLSAAVTLAVITVTPKSFGLPGSPQK
LLCEVVPASGEGRFVWRPLSDLSRSSEGPVLELQEAKLLAEQWQCQLYEGQKLLGATVYT
AESSSGAWSAKRISGDLKGGHLFLSLILGALALFLLVTGAFGFHLWRRQLLRRRFSALEH
GIRPPPVQSKIEELEREPETEMEPETEPDPEPQPEPELEPESRQL
SEQ ID NO: 71 (dog LAG-3)
MWEVQFLVLLLLQLLWVAPVAAPGSGTEVQVVWAQEGAPVQLPCSPTIPLQDVSLLRNAG
VTWYHLPESGPAAPALSLRPAAPSARGPGPRSYVVLMRAPGGLRSGLVPTVNALALNSPG
PTRFSLALQTVISLPHSWLFPGPSSLPGQPLASCWPSPHVGVGCVCEHPPFIHLTLLPAR
QSPLLFLLPSLQPQCSASLYLSLSASTLASSFPGLEPSGPLTVYTGAGSRVGLPCRLPPG
VGTQSFLTAKWTPPGGGPDLLVAGDDGNFTLQLEVVNQAQAGTYTCHIHLQGQQLSTTVT
LAVITVTPKSSGLPGNLRKLLCEVTPASGQERFVWSPLDKQSWRGSPGPCLEMQETRLLS
QPWQCHVYQAERLLGTAVYLIDPAGPGAQRSGRAQGVLKTGHLSLLLILGILFLLLLMTG
AFGFQLWRROWRPRRFSALELGTHPPQAQSKIGELEQEPELELEPEPELEPEPEPE
SEQ ID NO: 72 (mouse LAG-3)
MREDLLLGFLLLGLIWEAPVVSSGPGKELPVVWAQEGAPVHLPCSLKSPNLDPNFLRRGG
VIWQHQPDSGQPTPIPALDLHQGMPSPRQPAPGRYTVLSVAPGGLRSGRQPLHPHVQLEE
RGLQRGDFSLWLRPALRTDAGEYHATVRLPNRALSCSLRLRVGQASMIASPSGVLKLSDW
VLLNCSFSRPDRPVSVHWFQGQNRVPVYNSPRHFLAETFLLLPQVSPLDSGTWGCVLTYR
DGFNVSITYNLKVLGLEPVAPLTVYAAEGSRVELPCHLPPGVGTPSLLIAKWTPPGGGPE
LPVAGKSGNFTLHLEAVGLAQAGTYTCSIHLQGQQLNATVTLAVITVTPKSFGLPGSRGK
LLCEVTPASGKERFVWRPLNNLSRSCPGPVLEIQEARLLAERWQVQLYEGQRLLGATVYA
AESSSGAHSARRISGDLKGGHLVLVLILGALSLFLLVAGAFGFHWWRKQLLLRRFSALEH
GIQPFPAQRKIEELERELETEMGQEPEPEPEPQLEPEPRQL

1.-40. (canceled)

41. An anti-LAG-3 antibody or an antigen-binding portion thereof, wherein said antibody comprises the H-CDR1-3 and L-CDR1-3 amino acid sequences of:
a) SEQ ID NOs: 41, 42, 43, 44, 45, and 40, respectively;
b) SEQ ID NOs: 35, 36, 37, 38, 39, and 40, respectively;
c) SEQ ID NOs: 35, 42, 46, 44, 47, and 40, respectively;
d) SEQ ID NOs: 48, 49, 50, 51, 47, and 52, respectively;
e) SEQ ID NOs: 53, 54, 55, 44, 45, and 40, respectively;
f) SEQ ID NOs: 56, 57, 58, 59, 60, and 61, respectively; or
g) SEQ ID NOs: 62, 63, 64, 65, 66, and 67, respectively.

42. The anti-LAG-3 antibody or antigen-binding portion of claim 41, wherein
a) the heavy chain variable domain of said antibody is at least 90% identical in sequence to SEQ ID NO: 7 and the light chain variable domain of said antibody is at least 90% identical in sequence to SEQ ID NO: 8,
b) the heavy chain variable domain of said antibody is at least 90% identical in sequence to SEQ ID NO: 3 and the light chain variable domain of said antibody is at least 90% identical in sequence to SEQ ID NO: 4,
c) the heavy chain variable domain of said antibody is at least 90% identical in sequence to SEQ ID NO: 11 and the light chain variable domain of said antibody is at least 90% identical in sequence to SEQ ID NO: 12,
d) the heavy chain variable domain of said antibody is at least 90% identical in sequence to SEQ ID NO: 15 and the light chain variable domain of said antibody is at least 90% identical in sequence to SEQ ID NO: 16,
e) the heavy chain variable domain of said antibody is at least 90% identical in sequence to SEQ ID NO: 19 and the light chain variable domain of said antibody is at least 90% identical in sequence to SEQ ID NO: 20,
f) the heavy chain variable domain of said antibody is at least 90% identical in sequence to SEQ ID NO: 23 and the light chain variable domain of said antibody is at least 90% identical in sequence to SEQ ID NO: 24, or
g) the heavy chain variable domain of said antibody is at least 90% identical in sequence to SEQ ID NO: 27 and the light chain variable domain of said antibody is at least 90% identical in sequence to SEQ ID NO: 28.

43. The anti-LAG-3 antibody or antigen-binding portion of claim 41, wherein said antibody comprises a heavy chain variable domain and a light chain variable domain having the amino acid sequences of:
a) SEQ ID NOs: 7 and 8, respectively;
b) SEQ ID NOs: 3 and 4, respectively;
c) SEQ ID NOs: 11 and 12, respectively;
d) SEQ ID NOs: 15 and 16, respectively;
e) SEQ ID NOs: 19 and 20, respectively;
f) SEQ ID NOs: 23 and 24, respectively; or
g) SEQ ID NOs: 27 and 28, respectively.

44. The anti-LAG-3 antibody or antigen-binding portion of claim 41, wherein the antibody or antigen-binding portion has at least one property selected from:
a) at a concentration of 20 μg/mL, reduces the binding of human LAG-3 to human MHC class II on A375 cells by greater than 85% compared to a negative control antibody as determined by a flow cytometric competition assay;
b) at a concentration of 20 μg/mL, reduces the binding of human LAG-3 to human MHC class II on A375 cells to between 35% and 85% compared to a negative control antibody as determined by a flow cytometric competition assay;
c) blocks binding between human LAG-3 expressed on Jurkat cells and human MHC class II expressed on Raji cells;
d) binds to human LAG-3 with an EC₅₀ of 0.1 nM or less as measured by flow cytometry;
e) binds to cynomolgus LAG-3 with an EC₅₀ of 0.3 nM or less as measured by flow cytometry;
f) binds to human LAG-3 with a K_{D} of 3.0×10-8 M or less as measured by surface plasmon resonance;
g) binds to cynomolgus LAG-3 with a K_{D} of 1.5×10-7 M or less as measured by surface plasmon resonance;
h) binds to mouse LAG-3 with a K_{D} of 3.5×10-8 M or less as measured by surface plasmon resonance;
i) stimulates IL-2 production in Staphylococcal enterotoxin B (SEB) treated human peripheral blood mononuclear cells (PBMCs);
j) reduces cellular levels of LAG-3 in human T cells;
k) reduces soluble levels of LAG-3 in the culture of human T cells;
l) induces tumor growth regression in vivo;
m) delays tumor growth in vivo; and
n) does not bind to the same epitope of human LAG-3 as antibody 25F7-Lag3.5.

45. The anti-LAG-3 antibody or antigen-binding portion of claim 41, wherein the antibody or antigen-binding portion binds to an epitope of human LAG-3 having:
a) amino acid residues H85, P86, A87, P89, S91, W92, and G93 of SEQ ID NO: 68;
b) amino acid residues A40, Q41, P43, P46, P49, D52, T62, Q64, H65, Q66, P67, D68, G93, P94, P96, R98, Y99, T100, V101, P106, G107, R119, E124, R129, G130, D131, S133, R137, P138, D143, R148, and R163 of SEQ ID NO: 68;
c) amino acid residues A40, Q41, P43, P46, P49, D52, T62, Q64, H65, Q66, P67, D68, P96, Y99, T100, V101, P106, G107, R119, E124, R129, G130, D131, S133, R137, P138, D143, R148, and R163 of SEQ ID NO: 68;
d) amino acid residues G107, L109, R110, and S111 of SEQ ID NO: 68,
e) amino acid residues 98-105 of SEQ ID NO: 68;
f) amino acid residues 78-105 and 123-131 of SEQ ID NO: 68;
g) amino acid residues 23-30, 40-66, 88-105, 123-137, and 148-152 of SEQ ID NO: 68; or
h) amino acid residues 23-30, 40-66, 98-105, 118-137, and 148-161 of SEQ ID NO: 68.

46. The anti-LAG-3 antibody of any one of claims 41-45, wherein the antibody is of isotype IgG.

47. The anti-LAG-3 antibody of claim 46, wherein the antibody is of isotype IgG subclass IgG₁,
optionally, the antibody comprises at least one mutation in the F_{C} region.

48. The anti-LAG-3 antibody of claim 46, wherein
a) the antibody is of isotype IgG subclass IgG₁ and one or both of the amino acid residues at positions 234 and 235 are mutated from Leu to Ala, or
b) the antibody is of isotype IgG subclass IgG₄ and the amino acid residue at position 228 is mutated from Ser to Pro.

49. The anti-LAG-3 antibody or antigen-binding portion of claim 41, wherein said antibody comprises:
a) an HC with the amino acid sequences of SEQ ID NOs: 7 and 30 and an LC with the amino acid sequences of SEQ ID NOs: 8 and 34;
b) an HC with the amino acid sequences of SEQ ID NOs: 3 and 30 and an LC with the amino acid sequences of SEQ ID NOs: 4 and 34;
c) an HC with the amino acid sequences of SEQ ID NOs: 11 and 30 and an LC with the amino acid sequences of SEQ ID NOs: 12 and 34;
d) an HC with the amino acid sequences of SEQ ID NOs: 15 and 30 and an LC with the amino acid sequences of SEQ ID NOs: 16 and 34;
e) an HC with the amino acid sequences of SEQ ID NOs: 19 and 30 and an LC with the amino acid sequences of SEQ ID NOs: 20 and 34;
f) an HC with the amino acid sequences of SEQ ID NOs: 23 and 30 and an LC with the amino acid sequences of SEQ ID NOs: 24 and 34; or
g) an HC with the amino acid sequences of SEQ ID NOs: 27 and 30 and an LC with the amino acid sequences of SEQ ID NOs: 28 and 32.

50. An anti-LAG-3 antibody or an antigen-binding portion thereof, wherein said antibody competes for binding to human LAG-3 with the anti-LAG-3 antibody or antigen-binding portion thereof according to claim 41.

51. A pharmaceutical composition comprising the anti-LAG-3 antibody or antigen-binding portion thereof according to any one of claims 41-49 and a pharmaceutically acceptable excipient.

52. Isolated nucleic acid molecule(s) comprising a nucleotide sequence that encodes the heavy chain sequence, and a nucleotide sequence that encodes the light chain sequence, of the anti-LAG-3 antibody or antigen-binding portion of any one of claims 41-49.

53. Vector(s) comprising the isolated nucleic acid molecule(s) of claim 52, wherein said vector(s) further comprise an expression control sequence.

54. A host cell comprising a nucleotide sequence that encodes the heavy chain sequence, and a nucleotide sequence that encodes the light chain sequence, of the anti-LAG-3 antibody or antigen-binding portion of any one of claims 41-49.

55. A method for producing an anti-LAG-3 antibody or an antigen-binding portion thereof, comprising providing a host cell according to claim 54, culturing said host cell under conditions suitable for expression of the antibody or antigen-binding portion, and isolating the resulting antibody or antigen-binding portion.

56. A bi-specific binding molecule comprising the antigen-binding portion of an anti-LAG-3 antibody of any one of claims 41-49 and the antigen-binding portion of another, distinct antibody.

57. A immunoadhesion molecule comprising the anti-LAG-3 antibody or antigen-binding portion thereof of any one of claims 41-49 and one or more other proteins or peptides.

58. A method for treating cancer in a patient, comprising administering to said patient the antibody or antigen-binding portion according to any one of claims 41-49, a pharmaceutical composition according to claim 51, or a bi-specific binding molecule according to claim 56.

59. The method of claim 58, wherein the cancer originates in a tissue selected from the group consisting of skin, lung, intestine, colon, ovary, brain, prostate, kidney, soft tissues, hematopoietic system, head and neck, liver, bladder, breast, stomach, uterus, and pancreas;
optionally, the cancer is hematologic malignancies, fibrosarcoma, non-small cell lung cancer, melanoma, glioblastoma, gliosarcoma, colorectal cancer, , head and neck squamous cell cancer, renal cell carcinoma, Hodgkin's lymphoma, non-Hodgkin's lymphoma, glioma, small-cell lung cancer, hepatocellular carcinoma, bladder cancer, upper urinary tract cancer, esophageal cancer, gastroesophageal junction cancer, gastric cancer, liver cancer, multiple myeloma, sarcomas, myelodysplastic syndrome, nasopharyngeal cancer, small lymphocytic lymphoma, ovarian cancer, primary peritoneal cancer, fallopian tube cancer, urothelial cancer, prostate cancer, meningioma, adrenocortical cancer, kidney cancer, breast cancer, pancreatic cancer, endometrial cancer, skin basal cell cancer, cancer of the appendix, biliary tract cancer, salivary gland cancer, advanced Merkel cell cancer, diffuse large B cell lymphoma, follicular lymphoma, or mesothelioma;
optionally, the cancer at an early, intermediate, late, or metastatic stage.

60. The method of claim 59, further comprising administering to the patient an immunostimulatory agent, a vaccine, a chemotherapeutic agent, an anti-neoplastic agent, an anti-angiogenic agent, a tyrosine kinase inhibitor, a LAG-3 pathway inhibitor, radiation therapy, vaccines, cytokines, T cell therapies, retinoic acid, phenylbutyrate, all-trans-retinoic acid, or active form vitamin D.
